(19) United States
(12) Patent Application Publication
(10) Pub. No.: US 2022/0348933 A1
(43) Pub. Date: Nov. 3, 2022

(54) BIOSYNTHESIS OF ENZYMES FOR USE IN TREATMENT OF MAPLE SYRUP URINE DISEASE (MSUD)

(71) Applicants: Ginkgo Bioworks, Inc., Boston, MA (US); Synlogic Operating Company, Inc., Cambridge, MA (US)

(72) Inventors: Patrick Boyle, Boston, MA (US); Dylan Alexander Carlin, Boston, MA (US); Rishi Jain, Boston, MA (US); Ryan Putman, Boston, MA (US); Laura Stone, Boston, MA (US); Alex C. Tucker, Boston, MA (US); Kolea Zimmerman, Boston, MA (US)

(73) Assignees: Ginkgo Bioworks, Inc., Boston, MA (US); Synlogic Operating Company, Inc., Cambridge, MA (US)

(21) Appl. No.: 17/621,214

(22) PCT Filed: Jun. 19, 2020
(86) PCT No.: PCT/US2020/038813

§ 371 Date: Dec. 20, 2021

Related U.S. Application Data
(60) Provisional application No. 62/864,875, filed on Jun. 21, 2019.
(60) Provisional application No. 62/865,129, filed on Jun. 21, 2019.

(51) Int. Cl. C12N 15/52; C12N 15/70

(52) U.S. Cl. CPC C12N 15/52; C12N 15/70

(57) ABSTRACT

Provided in this disclosure, in some embodiments, are methods and compositions for treating maple syrup urine disease (MSUD) and other conditions characterized by excessive branched-chain amino acids.

### CROSS-REFERENCE TO RELATED APPLICATIONS

This application claims the benefit under 35 U.S.C. § 119(e) of U.S. Provisional Application Ser. No. 62/865,129, filed Jun. 21, 2019, entitled “BIOSYNTHESIS OF ENZYMES FOR USE IN TREATMENT OF MAPLE SYRUP URINE DISEASE (MSUD),” and U.S. Provisional Application Ser. No. 62/864,875, filed Jun. 21, 2019, entitled “OPTIMIZED BACTERIA ENGINEERED TO TREAT DISORDERS INVOLVING THE CATABOLISM OF LEUCINE, ISOLEUCINE, AND/OR VALINE,” the disclosure of each which is incorporated by reference herein in its entirety.

### REFERENCE TO A SEQUENCE LISTING SUBMITTED AS A TEXT FILE VIA EFS-WEB

The instant application contains a Sequence Listing which has been submitted in ASCII format via EFS-Web and is hereby incorporated by reference in its entirety. Said ASCII copy, created on Jun. 19, 2020, is named G0919.70033WO00-SEQ-OMJ.txt, and is 1.76 megabytes (MB) in size.

### FIELD OF INVENTION

The present disclosure relates to enzymes, nucleic acids, and cells useful for the conversion of leucine to isopentanol.

### BACKGROUND

Maple syrup urine disease (MSUD) is a metabolic disorder caused by a deficiency of the branched-chain alpha-keto acid dehydrogenase complex (BCKDC), leading to a buildup of the branched-chain amino acids (leucine, isoleucine, and valine) and their toxic by-products (ketoacids) in the blood and urine. MSUD gets its name from the distinctive sweet odor of affected individual's urine, particularly prior to diagnosis, and during times of acute illness. There remains a need for improved treatments for MSUD and other conditions characterized by excessive branched-chain amino acids.

### SUMMARY

The present disclosure is based, at least in part, on generation of engineered cells containing enzymes for consuming leucine, for example, by converting leucine to isopentanol. Such engineered cells are useful, e.g., to treat diseases associated with accumulation of leucine such as MSUD.

Aspects of the disclosure relate to host cells that comprise a heterologous polynucleotide encoding a leucine dehydrogenase (LeuDH) enzyme, wherein the LeuDH enzyme comprises an amino acid sequence that is at least 90% identical to a sequence selected from SEQ ID NOs: 2, 4, 6, 8, 10, and 12. In some embodiments, the LeuDH enzyme comprises an amino acid sequence that is at least 90% identical to SEQ ID NO: 2. In some embodiments, the LeuDH enzyme comprises SEQ ID NO: 2. In some embodiments, the LeuDH enzyme comprises: V at a residue corresponding to residue 13 in SEQ ID NO: 27; W at a residue corresponding to residue 16 in SEQ ID NO: 27; Q at a residue corresponding to residue 42 in SEQ ID NO: 27; T, Y, F, E, or W at a residue corresponding to residue 43 in SEQ ID NO: 27; I, H, K, or Y at a residue corresponding to residue 44 in SEQ ID NO: 27; T, E, A, S, or K at a residue corresponding to residue 67 in SEQ ID NO: 27; K at a residue corresponding to residue 71 in SEQ ID NO: 27; S at a residue corresponding to residue 73 in SEQ ID NO: 27; R, H, Y, S, K, or W at a residue corresponding to residue 76 in SEQ ID NO: 27; Y at a residue corresponding to residue 92 in SEQ ID NO: 27; H at a residue corresponding to residue 93 in SEQ ID NO: 27; G at a residue corresponding to residue 95 in SEQ ID NO: 27; G at a residue corresponding to residue 100 in SEQ ID NO: 27; C at a residue corresponding to residue 105 in SEQ ID NO: 27; G at a residue corresponding to residue 111 in SEQ ID NO: 27; M at a residue corresponding to residue 113 in SEQ ID NO: 27; N, or V at a residue corresponding to residue 115 in SEQ ID NO: 27; R, N, or W at a residue corresponding to residue 116 in SEQ ID NO: 27; A at a residue corresponding to residue 120 in SEQ ID NO: 27; D at a residue corresponding to residue 122 in SEQ ID NO: 27; E at a residue corresponding to residue 136 in SEQ ID NO: 27; D at a residue corresponding to residue 140 in SEQ ID NO: 27; M at a residue corresponding to residue 141 in SEQ ID NO: 27; S at a residue corresponding to residue 160 in SEQ ID NO: 27; F at a residue corresponding to residue 185 in SEQ ID NO: 27; N at a residue corresponding to residue 196 in SEQ ID NO: 27; Y at a residue corresponding to residue 228 in SEQ ID NO: 27; M at a residue corresponding to residue 248 in SEQ ID NO: 27; C at a residue corresponding to residue 256 in SEQ ID NO: 27; Q or C at a residue corresponding to residue 293 in SEQ ID NO: 27; K or N at a residue corresponding to residue 296 in SEQ ID NO: 27; R, Q, or K at a residue corresponding to residue 297 in SEQ ID NO: 27; C or D at a residue corresponding to residue 300 in SEQ ID NO: 27; T or S at a residue corresponding to residue 302 in SEQ ID NO: 27; C at a residue corresponding to residue 305 in SEQ ID NO: 27; F at a residue corresponding to residue 319 in SEQ ID NO: 27; and/or M at a residue corresponding to residue 330 in SEQ ID NO: 27.

Further aspects of the disclosure relate to host cells that comprise a heterologous polynucleotide encoding a leucine dehydrogenase (LeuDH) enzyme, wherein the LeuDH enzyme comprises: V at a residue corresponding to residue 13 in SEQ ID NO: 27; W at a residue corresponding to residue 16 in SEQ ID NO: 27; Q at a residue corresponding to residue 42 in SEQ ID NO: 27; T, Y, F, E, or W at a residue corresponding to residue 43 in SEQ ID NO: 27; I, H, K, or Y at a residue corresponding to residue 44 in SEQ ID NO: 27; T, E, A, S, or K at a residue corresponding to residue 67 in SEQ ID NO: 27; K at a residue corresponding to residue 71 in SEQ ID NO: 27; S at a residue corresponding to residue 73 in SEQ ID NO: 27; R, H, Y, S, K, or W at a residue corresponding to residue 76 in SEQ ID NO: 27; Y at a residue corresponding to residue 92 in SEQ ID NO: 27; H at a residue corresponding to residue 93 in SEQ ID NO: 27; G at a residue corresponding to residue 95 in SEQ ID NO: 27; G at a residue corresponding to residue 100 in SEQ ID NO: 27; C at a residue corresponding to residue 105 in SEQ ID NO: 27; G at a residue corresponding to residue 111 in SEQ ID NO: 27; M at a residue corresponding to residue 113 in SEQ ID NO: 27; N, or V at a residue corresponding to residue 115 in SEQ ID NO: 27; R, N, or W at a residue corresponding to residue 116 in SEQ ID NO: 27; A at a residue corresponding to residue 120 in SEQ ID NO: 27; D at a residue corresponding to residue 122 in SEQ ID NO: 27; E at a residue corresponding to residue 136 in SEQ ID NO: 27; D at a residue corresponding to residue 140 in SEQ ID NO: 27; M at a residue corresponding to residue 141 in SEQ ID NO: 27; S at a residue corresponding to residue 160 in SEQ ID NO: 27; F at a residue corresponding to residue 185 in SEQ ID NO: 27; N at a residue corresponding to residue 196 in SEQ ID NO: 27; Y at a residue corresponding to residue 228 in SEQ ID NO: 27; M at a residue corresponding to residue 248 in SEQ ID NO: 27; C at a residue corresponding to residue 256 in SEQ ID NO: 27; Q or C at a residue corresponding to residue 293 in SEQ ID NO: 27; K or N at a residue corresponding to residue 296 in SEQ ID NO: 27; R, Q, or K at a residue corresponding to residue 297 in SEQ ID NO: 27; C or D at a residue corresponding to residue 300 in SEQ ID NO: 27; T or S at a residue corresponding to residue 302 in SEQ ID NO: 27; C at a residue corresponding to residue 305 in SEQ ID NO: 27; F at a residue corresponding to residue 319 in SEQ ID NO: 27; and M at a residue corresponding to residue 330 in SEQ ID NO: 27.

Further aspects of the disclosure relate to host cells that comprise a heterologous polynucleotide encoding a leucine dehydrogenase (LeuDH) enzyme, wherein relative to SEQ ID NO: 27, the LeuDH enzyme comprises an amino acid substitution at amino acid residue: 42, 43, 44, 67, 71, 76, 78, 113, 115, 116, 136, 293, 296, 297 and/or 300. In some embodiments, the LeuDH enzyme comprises: A, Q, or T at residue 42; E, F, T, W, or Y at residue 43; H, I, K, or Y at residue 44; A, E, K, Q, S, or T at residue 67; C, D, H, K, M, or T at residue 71; E, F, H, I, K, M, R, S, T, W, or Y at residue 76; C, F, H, K, Q, V, or Y at residue 78; F, M, Q, V, W, or Y at residue 113; N, Q, S, T, or V at residue 115; A, L, M, N, R, S, V, or W at residue 116; E, F, L, R, S, or Y at residue 136; A, C, Q, S, or T at residue 293; A, C, E, I, K, L, N, S, or T at residue 296; C, D, E, F, H, K, L, M, N, Q, R, T, W, or Y at residue 297; and/or A, C, D, F, H, K, M, N, Q, R, S, T, W, or Y at residue 300.

Further aspects of the present disclosure relate to non-naturally occurring LeuDH enzymes, wherein relative to SEQ ID NO: 27, the LeuDH enzyme comprises an amino acid substitution at amino acid residue: 42, 43, 44, 67, 71, 76, 78, 113, 115, 116, 136, 293, 296, 297 and/or 300. In some embodiments, the LeuDH enzyme comprises: A, Q, or T at residue 42; E, F, T, W, or Y at residue 43; H, I, K, or Y at residue 44; A, E, K, Q, S, or T at residue 67; C, D, H, K, M, or Tat residue 71; E, F, H, I, K, M, R, S, T, W, or Y at residue 76; C, F, H, K, Q, V, or Y at residue 78; F, M, Q, V, W, or Y at residue 113; N, Q, S, T, or V at residue 115; A, L, M, N, R, S, V, or W at residue 116; E, F, L, R, S, or Y at residue 136; A, C, Q, S, or T at residue 293; A, C, E, I, K, L, N, S, or T at residue 296; C, D, E, F, H, K, L, M, N, Q, R, T, W, or Y at residue 297; and/or A, C, D, F, H, K, M, N, Q, R, S, T, W, or Y at residue 300.

Further aspects of the disclosure relate to host cells that comprise a heterologous polynucleotide encoding a branched chain α-ketoacid decarboxylase (KivD) enzyme, wherein the KivD enzyme comprises an amino acid sequence that is at least 90% identical to a sequence selected from SEQ ID NOs: 14, 16, and 18. In some embodiments, the KivD enzyme comprises an amino acid sequence that is at least 90% identical to SEQ ID NO: 18. In some embodiments, the KivD enzyme comprises SEQ ID NO: 18. In some embodiments, the KivD enzyme comprises: Y at a residue corresponding to residue 33 in SEQ ID NO: 29; Q at a residue corresponding to residue 44 in SEQ ID NO: 29; M at a residue corresponding to residue 117 in SEQ ID NO: 29; I at a residue corresponding to residue 129 in SEQ ID NO: 29; W at a residue corresponding to residue 185 in SEQ ID NO: 29; I at a residue corresponding to residue 190 in SEQ ID NO: 29; I at a residue corresponding to residue 225 in SEQ ID NO: 29; Y at a residue corresponding to residue 227 in SEQ ID NO: 29; L at a residue corresponding to residue 311 in SEQ ID NO: 29; G at a residue corresponding to residue 312 in SEQ ID NO: 29; T at a residue corresponding to residue 313 in SEQ ID NO: 29; P at a residue corresponding to residue 328 in SEQ ID NO: 29; W at a residue corresponding to residue 341 in SEQ ID NO: 29; H at a residue corresponding to residue 345 in SEQ ID NO: 29; C at a residue corresponding to residue 347 in SEQ ID NO: 29; R at a residue corresponding to residue 420 in SEQ ID NO: 29; D at a residue corresponding to residue 494 in SEQ ID NO: 29; C at a residue corresponding to residue 508 in SEQ ID NO: 29; and/or F at a residue corresponding to residue 550 in SEQ ID NO: 29.

Further aspects of the disclosure relate to host cells that comprise a heterologous polynucleotide encoding a branched chain α-ketoacid decarboxylase (KivD) enzyme, wherein the KivD enzyme comprises: Y at a residue corresponding to residue 33 in SEQ ID NO: 29; Q at a residue corresponding to residue 44 in SEQ ID NO: 29; M at a residue corresponding to residue 117 in SEQ ID NO: 29; I at a residue corresponding to residue 129 in SEQ ID NO: 29; W at a residue corresponding to residue 185 in SEQ ID NO: 29; I at a residue corresponding to residue 190 in SEQ ID NO: 29; I at a residue corresponding to residue 225 in SEQ ID NO: 29; Y at a residue corresponding to residue 227 in SEQ ID NO: 29; L at a residue corresponding to residue 311 in SEQ ID NO: 29; G at a residue corresponding to residue 312 in SEQ ID NO: 29; T at a residue corresponding to residue 313 in SEQ ID NO: 29; P at a residue corresponding to residue 328 in SEQ ID NO: 29; W at a residue corresponding to residue 341 in SEQ ID NO: 29; H at a residue corresponding to residue 345 in SEQ ID NO: 29; C at a residue corresponding to residue 347 in SEQ ID NO: 29; R at a residue corresponding to residue 420 in SEQ ID NO: 29; D at a residue corresponding to residue 494 in SEQ ID NO: 29; C at a residue corresponding to residue 508 in SEQ ID NO: 29; and F at a residue corresponding to residue 550 in SEQ ID NO: 29.

Further aspects of the disclosure relate to host cells that comprise a heterologous polynucleotide encoding an alcohol dehydrogenase (Adh) enzyme wherein the Adh enzyme comprises an amino acid sequence that is at least 90% identical to a sequence selected from SEQ ID NOs: 20, 22, and 24. In some embodiments, the Adh enzyme comprises an amino acid sequence that is at least 90% identical to SEQ ID NO: 24. In some embodiments, the Adh enzyme comprises SEQ ID NO: 24. In some embodiments, the Adh enzyme comprises: P at a residue corresponding to residue 9 in SEQ ID NO: 31; G at a residue corresponding to residue 16 in SEQ ID NO: 31; Q at a residue corresponding to residue 23 in SEQ ID NO: 31; R at a residue corresponding to residue 28 in SEQ ID NO: 31; A at a residue corresponding to residue 30 in SEQ ID NO: 31; K at a residue corresponding to residue 93 in SEQ ID NO: 31; L at a residue corresponding to residue 98 in SEQ ID NO: 31; R at a residue corresponding to residue 99 in SEQ ID NO: 31; P at a residue corresponding to residue 114 in SEQ ID NO: 31; K at a residue corresponding to residue 115 in SEQ ID NO: 31; Y at a residue corresponding to residue 119 in SEQ ID NO: 31; Y at a residue corresponding to residue 194 in SEQ ID NO: 31; P at a residue corresponding to residue 242 in SEQ ID NO: 31; K at a residue corresponding to residue 249 in SEQ ID NO: 31; E at a residue corresponding to residue 255 in SEQ ID NO: 31; D at a residue corresponding to residue 260 in SEQ ID NO: 31; H at a residue corresponding to residue 269 in SEQ ID NO: 31; Q at a residue corresponding to residue 281 in SEQ ID NO: 31; L at a residue corresponding to residue 325 in SEQ ID NO: 31; M at a residue corresponding to residue 333 in SEQ ID NO: 31; P at a residue corresponding to residue 334 in SEQ ID NO: 31; and/or Q at a residue corresponding to residue 348 in SEQ ID NO: 31.

Further aspects of the disclosure relate to host cells that comprises a heterologous polynucleotide encoding a an alcohol dehydrogenase (Adh) enzyme, wherein the Adh enzyme comprises: P at a residue corresponding to residue 9 in SEQ ID NO: 31; G at a residue corresponding to residue 16 in SEQ ID NO: 31; Q at a residue corresponding to residue 23 in SEQ ID NO: 31; R at a residue corresponding to residue 28 in SEQ ID NO: 31; A at a residue corresponding to residue 30 in SEQ ID NO: 31; K at a residue corresponding to residue 93 in SEQ ID NO: 31; L at a residue corresponding to residue 98 in SEQ ID NO: 31; R at a residue corresponding to residue 99 in SEQ ID NO: 31; P at a residue corresponding to residue 114 in SEQ ID NO: 31; K at a residue corresponding to residue 115 in SEQ ID NO: 31; Y at a residue corresponding to residue 119 in SEQ ID NO: 31; Y at a residue corresponding to residue 194 in SEQ ID NO: 31; P at a residue corresponding to residue 242 in SEQ ID NO: 31; K at a residue corresponding to residue 249 in SEQ ID NO: 31; E at a residue corresponding to residue 255 in SEQ ID NO: 31; D at a residue corresponding to residue 260 in SEQ ID NO: 31; H at a residue corresponding to residue 269 in SEQ ID NO: 31; Q at a residue corresponding to residue 281 in SEQ ID NO: 31; L at a residue corresponding to residue 325 in SEQ ID NO: 31; M at a residue corresponding to residue 333 in SEQ ID NO: 31; P at a residue corresponding to residue 334 in SEQ ID NO: 31; and Q at a residue corresponding to residue 348 in SEQ ID NO: 31.

In some embodiments, the host cell is a plant cell, an algal cell, a yeast cell, a bacterial cell, or an animal cell. In some embodiments, the host cell is a yeast cell. In some embodiments, the yeast cell is a Saccharomyces cell, a Yarrowia cell or a Pichia cell. In some embodiments, the host cell is a bacterial cell. In some embodiments, the bacterial cell is an E. coli cell or a Bacillus cell.

In some embodiments, the host cell further comprises a heterologous polynucleotide encoding a branched-chain amino acid transport system 2 carrier protein (BrnQ). In some embodiments, the BrnQ protein is at least 90% identical to the amino acid sequence of SEQ ID NO: 35. In some embodiments, the BrnQ protein comprises the amino acid sequence of SEQ ID NO: 35.

In some embodiments, the heterologous polynucleotide is operably linked to an inducible promoter. In some embodiments, the heterologous polynucleotide is expressed in an operon. In some embodiments, the operon expresses more than one heterologous polynucleotide, and a ribosome binding site may be present between each heterologous polynucleotide.

In some embodiments, the host cell further comprises a heterologous polynucleotide encoding a KivD enzyme and/or a heterologous polynucleotide encoding an Adh enzyme.

In some embodiments, the host cell further comprises a heterologous polynucleotide encoding a LeuDH enzyme and/or a heterologous polynucleotide encoding an Adh enzyme.

In some embodiments, the host cell further comprises a heterologous polynucleotide encoding a LeuDH enzyme and/or a heterologous polynucleotide encoding a KivD enzyme.

In some embodiments, the host cell is capable of producing isopentanol from leucine. In some embodiments, the host cell consumes at least two-fold more leucine relative to a control host cell that comprises a heterologous polynucleotide encoding a control LeuDH enzyme comprising the sequence of SEQ ID NO: 27, a heterologous polynucleotide encoding a control KivD enzyme comprising the sequence of SEQ ID NO: 29, a heterologous polynucleotide encoding a control Adh enzyme comprising the sequence of SEQ ID NO: 31, and a heterologous polynucleotide encoding a control BrnQ protein comprising the sequence of SEQ ID NO: 35.

Further aspects of the disclosure relate to methods comprising culturing any of the host cells disclosed in this application.

Further aspects of the disclosure relate to methods for producing isopentanol from leucine comprising culturing any of the host cells disclosed in this application.

Further aspects of the disclosure relate to non-naturally occurring nucleic acids comprising a sequence that is at least 90% identical to a nucleic acid sequence selected from SEQ ID NOs: 1, 3, 5, 7, 9, and 11.

Further aspects of the disclosure relate to non-naturally occurring nucleic acids comprising a sequence that is at least 90% identical to a nucleic acid sequence selected from SEQ ID NOs: 13, 15, and 17.

Further aspects of the disclosure relate to non-naturally occurring nucleic acids comprising a sequence that is at least 90% identical to a nucleic acid sequence selected from SEQ ID NOs: 19, 21, and 23.

Further aspects of the disclosure relate to non-naturally occurring nucleic acids encoding a sequence that is at least 90% identical to a sequence selected from SEQ ID NOs: 2, 4, 6, 8, 10, and 12.

Further aspects of the disclosure relate to non-naturally occurring nucleic acids encoding a sequence that is at least 90% identical to a sequence selected from SEQ ID NOs: 14, 16, and 18.

Further aspects of the disclosure relate to non-naturally occurring nucleic acids encoding a sequence that is at least 90% identical to a sequence selected from SEQ ID NOs: 20, 22, and 24.

Further aspects of the disclosure relate to vectors comprising any of the non-naturally occurring nucleic acids disclosed in this application.

Further aspects of the disclosure relate to expression cassettes comprising any of the non-naturally occurring nucleic acids disclosed in this application.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways.

### BRIEF DESCRIPTION OF THE DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIGS. 1A-1C depict sequence similarity networks. Each spot represents a single amino acid sequence available in sequence databases. The more closely-related amino acid sequences are, the closer the spots are to one another. Each sequence similarity network has a corresponding cluster key with information regarding the annotation or source of the enzyme. FIG. 1A shows a sequence similarity network for leucine dehydrogenase (LeuDH). The cluster key indicates the annotation of the enzyme. FIG. 1B shows a sequence similarity network for ketoisovalerate decarboxylase (KivD). The annotation each spot represents the phylogenetic clade from which the enzyme was sourced. FIG. 1C shows a sequence similarity network for alcohol dehydrogenase (Adh). The annotation of each spot represents the phylogenetic clade from which the enzyme was sourced.

FIG. 2 depicts a graph showing data from screening of LeuDH enzymes. 220 LeuDH enzymes were screened with biological replication (n=4) to validate enzyme activity and ranking. Activities are reported relative the B. cereus LeuDH activity.

FIG. 3 depicts graphs showing data from comparison of activity and specificity of LeuDH enzymes. The top˜200 LeuDH enzymes were screened for activity on Leu, Val, and Ile. Activity of LeuDH enzymes on Leu are reported relative to B. cereus LeuDH activity. Specificity is measured as the ratio of activity on Leu relative to Val/Leu. In the left panel, enzyme activity on Leu is reported relative to the Leu/Val specificity. In the right panel, enzyme activity is reported relative to the Leu/Ile specificity. Rationally engineered active site variants are shown as unfilled circles. Sourced LeuDH enzymes are shown in solid filled circles. The negative control and positive control B. cereus LeuDH are also shown.

FIG. 4 shows data from comparison of specificity for LeuDH enzymes. The top˜200 LeuDH enzymes were screened for activity on Leu, Val, and Be. Specificity is measured as the ratio of activity on Leu relative to Val/Leu. Rationally engineered active site variants are shown as unfilled circles. Sourced LeuDH enzymes are shown with filled circles. The negative control and the positive control B. cereus LeuDH are shown.

FIG. 5 depicts a graph showing data from screening of KivD enzymes. 55 KivD enzymes were screened for activity with biological replication (n=4). Activities are reported relative to the activity of a lysate containing heterologously expressed S. aureus KivD (whose activity was indistinguishable from the measurable background activity of the lysate and so was equated to background).

FIG. 6 shows data from screening of Adh enzymes. 55 Adh enzymes were screened with biological replication (n=4). Activities are reported relative to the activity of a lysate containing heterologously expressed S. cerevisiae ADH2 (whose activity was indistinguishable from the measurable background activity of the lysate and so was equated to background).

FIG. 7 shows data of selectivity of LeuDH enzymes. In total, 21 candidate LeuDH enzymes were tested. Each set of bars, from left to right, shows Leu consumed, Be consumed and Val consumed.

FIG. 8 shows a comparison of the rate of Leu consumption over time between top Leu consuming strains (5941, 5942 and 5943) and a prototype strain (1980). 8 mM leucine was added to minimum media and samples were taken at 0, 2, and 4 hour time points after anaerobic incubation.

FIG. 9 shows the MSUD pathway for conversion of leucine to isopentanol.

FIG. 10 shows extracellular profiles of the isopentanol pathway intermediates for strain 5941 assayed in Ambr15 bioreactors (n=2). Error bars reflect standard deviation across the duplicate bioreactors. The data corresponding to “Sum” represents the aggregate total concentration of the intermediates shown. Leu=Leucine, Acid=2-oxoisocaproate, Aldehyde=isovaleraldehyde, Alcohol=isopentanol.

### DETAILED DESCRIPTION OF THE INVENTION

The present disclosure provides, in some aspects, cells and combination of enzymes of the branched-chain amino acid (BCAA) pathway that are engineered for leucine consumption. These BCAA pathway enzymes include leucine dehydrogenase (LeuDH), ketoisovalerate decarboxylase (KivD), and alcohol dehydrogenase (Adh). The disclosed enzymes and host cells comprising such enzymes may be used to promote leucine consumption, e.g., in a subject suffering from a disorder associated with a buildup of BCAA (e.g., leucine) such as maple syrup urine disease (MSUD) and in other medical and industrial settings.

### Leucine Dehydrogenase (LeuDH)

As used in this disclosure “leucine dehydrogenase (LeuDH)” refers to an enzyme that catalyzes the reversible deamination of branched-chain L-amino acids (e.g., L-leucine, L-valine, L-isoleucine) to their 2-oxo analogs. A LeuDH enzyme may use L-leucine as a substrate. In some embodiments, LeuDH exhibits specificity for L-leucine compared to L-valine and/or L-isoleucine. In some embodiments, LeuDH produces ketoisocaproate (also known as 2-oxoisocaproate) from L-leucine.

In some embodiments, a host cell comprises a LeuDH enzyme and/or a heterologous polynucleotide encoding such an enzyme. In some embodiments, a host cell comprises a heterologous polynucleotide encoding a LeuDH enzyme comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to any one of SEQ ID NO: 2, 4, 6, 8, 10, 12, or 257-475, a LeuDH enzyme in Table 3 or Table 4, or a LeuDH enzyme otherwise described in this disclosure. In some embodiments, a host cell comprises a heterologous polynucleotide that is at least 90% (e.g., at least 90%, at least 91%, at least 92%, at least 93%, at least 94%, at least 95%, at least 96%, at least 97%, at least 98%, at least 99%, or 100%) identical to any one of SEQ ID NO: 1, 3, 5, 7, 9, 11, or 37-255, a polynucleotide encoding a LeuDH enzyme in Table 3 or Table 4, or a LeuDH enzyme otherwise described in this disclosure.

In some embodiments, a host cell comprises LeuDH from Bacillus cereus. In other embodiments, a host cell does not comprise LeuDH from Bacillus cereus.

LeuDH from Bacillus cereus can comprise the amino acid sequence of UniProtKB—P0A392 (SEQ ID NO: 27):

(SEQ ID NO: 27)
MTLEIFEYLEKYDYEQVVFCQDKESGLKAIIAIHDTTLGPALGGTRMWTY
DSEEAAIEDALRLAKGMTYKNAAAGLNLGGAKTVIIGDPRKDKSEAMFRA
LGRYIQGLNGRYITAEDVGTTVDDMDIIHEETDFVTGISPSFGSSGNPSP
VTAYGVYRGMKAAAKEAFGTDNLEGKVIAVQGVGNVAYHLCKHLHAEGAK
LIVTDINKEAVQRAVEEFGASAVEPNEIYGVECDIYAPCALGATVNDETI
PQLKAKVIAGSANNQLKEDRHGDIIHEMGIVYAPDYVINAGGVINVADEL
YGYNRERALKRVESIYDTIAKVIEISKRDGIATYVAADRLAEERIASLKN
SRSTYLRNGHDIISRR

In some embodiments, the amino acid sequence of SEQ ID NO: 27 is encoded by the nucleic acid sequence:

(SEQ ID NO: 28)
ATGACCCTTGAGATTTTTGAATACCTCGAAAAATATGATTATGAGCAGGT
CGTTTTCTGTCAAGACAAGGAATCAGGACTGAAAGCGATCATTGCTATCC
ATGATACTACACTGGGGCCAGCCTTAGGTGGCACCCGTATGTGGACGTAC
GACTCGGAAGAAGCGGCAATTGAGGATGCCTTGAGGTTAGCTAAGGGCAT
GACGTATAAAAACGCGGCAGCCGGTTTGAATCTGGGCGGTGCGAAAACCG
TGATTATCGGGGATCCCCGCAAAGACAAATCTGAAGCAATGTTTCGGGCG
CTGGGCCGATACATACAGGGACTAAATGGTCGCTATATCACCGCTGAAGA
TGTAGGAACTACCGTGGATGATATGGACATAATTCACGAAGAAACGGACT
TCGTCACGGGCATTAGCCCTAGTTTTGGTAGCTCCGGGAACCCGTCTCCG
GTTACCGCCTATGGCGTGTACCGTGGCATGAAGGCAGCAGCGAAAGAGGC
CTTTGGTACAGACAACCTGGAGGGGAAAGTGATCGCGGTTCAAGGGGTAG
GTAATGTGGCGTATCATCTGTGCAAACACTTACATGCCGAGGGCGCCAAG
CTGATTGTCACGGATATCAACAAAGAAGCGGTACAGCGTGCAGTCGAAGA
ATTTGGCGCTTCCGCCGTTGAGCCGAATGAAATCTACGGCGTGGAATGCG
ATATTTACGCGCCGTGTGCTCTTGGTGCGACAGTCAACGATGAAACGATC
CCTCAGCTGAAAGCAAAGGTAATTGCGGGTTCGGCTAATAACCAGTTAAA
AGAAGACAGACATGGAGACATAATTCACGAGATGGGTATTGTTTATGCAC
CAGATTATGTAATCAATGCGGGCGGCGTTATTAACGTCGCAGATGAACTG
TATGGCTACAACCGCGAACGCGCCCTCAAACGTGTGGAGTCAATTTATGA
CACCATTGCCAAAGTGATCGAAATCAGCAAGCGCGATGGAATCGCCACTT
ATGTGGCTGCCGATCGTCTGGCGGAAGAACGCATTGCAAGTCTCAAAAAT
AGCCGTTCCACCTACCTTCGCAATGGCCATGATATTATAAGTCGGCGTTG
A

In some embodiments, a host cell that expresses a heterologous polynucleotide encoding a LeuDH enzyme may increase conversion of leucine to ketoisocaproate by 0.5-fold, 1-fold, 1.5-fold, 2-fold, 2.5-fold, 3-fold, 3.5-fold, 4-fold, 4.5-fold, 5-fold, 5.5-fold, or 6-fold more (e.g., 2-fold to 6-fold more) relative to a control. In some embodiments, the control is a host cell that expresses a heterologous polynucleotide encoding SEQ ID NO: 27. In some embodiments, the control is an E. coli Nissle strain SYN1980 ΔleuE, ΔilvC, lacZ:tetR-Ptet-livKHMGF, tetR-Ptet-leuDH(Bc)-kivD-adh2-brnQ-rrnB ter (pSC101), such as is described in U.S. Patent Application Publication No. US20170232043.

In some embodiments, a host cell that expresses a heterologous polynucleotide encoding a LeuDH enzyme may exhibit at least 0.5-fold, 1-fold, 1.5-fold, 2-fold, 2.5-fold, 3-fold, 3.5-fold, 4-fold, 4.5-fold, 5-fold, 5.5-fold, or 6-fold more (e.g., 2-fold to 6-fold more) more activity on leucine relative to valine. In some embodiments, a host cell that expresses a heterologous polynucleotide encoding a LeuDH enzyme may exhibit at least 0.5-fold, 1-fold, 1.5-fold, 2-fold, 2.5-fold, 3-fold, 3.5-fold, 4-fold, 4.5-fold, 5-fold, 5.5-fold, or 6-fold more (e.g., 2-fold to 6-fold more) more activity on leucine relative to isoleucine.

In some embodiments, a LeuDH comprises a sequence that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or is 100% identical to SEQ ID NO: 27, any one of SEQ ID NO: 2, 4, 6, 8, 10, 12, or 257-475, any one of SEQ ID NO: 1, 3, 5, 7, 9, 11, or 37-255, an amino acid or polynucleotide sequence of a LeuDH enzyme in Table 3 or Table 4, or a LeuDH enzyme otherwise described in this disclosure.

In some embodiments, such a LeuDH enzyme comprises: V at a residue corresponding to residue 13 in SEQ ID NO: 27; W at a residue corresponding to residue 16 in SEQ ID NO: 27; Q at a residue corresponding to residue 42 in SEQ ID NO: 27; T, Y, F, E, or W at a residue corresponding to residue 43 in SEQ ID NO: 27; I, H, K, or Y at a residue corresponding to residue 44 in SEQ ID NO: 27; T, E, A, S, or K at a residue corresponding to residue 67 in SEQ ID NO: 27; K at a residue corresponding to residue 71 in SEQ ID NO: 27; S at a residue corresponding to residue 73 in SEQ ID NO: 27; R, H, Y, S, K, or W at a residue corresponding to residue 76 in SEQ ID NO: 27; Y at a residue corresponding to residue 92 in SEQ ID NO: 27; H at a residue corresponding to residue 93 in SEQ ID NO: 27; G at a residue corresponding to residue 95 in SEQ ID NO: 27; G at a residue corresponding to residue 100 in SEQ ID NO: 27; C at a residue corresponding to residue 105 in SEQ ID NO: 27; G at a residue corresponding to residue 111 in SEQ ID NO: 27; M at a residue corresponding to residue 113 in SEQ ID NO: 27; N, or V at a residue corresponding to residue 115 in SEQ ID NO: 27; R, N, or W at a residue corresponding to residue 116 in SEQ ID NO: 27; A at a residue corresponding to residue 120 in SEQ ID NO: 27; D at a residue corresponding to residue 122 in SEQ ID NO: 27; E at a residue corresponding to residue 136 in SEQ ID NO: 27; D at a residue corresponding to residue 140 in SEQ ID NO: 27; M at a residue corresponding to residue 141 in SEQ ID NO: 27; S at a residue corresponding to residue 160 in SEQ ID NO: 27; F at a residue corresponding to residue 185 in SEQ ID NO: 27; N at a residue corresponding to residue 196 in SEQ ID NO: 27; Y at a residue corresponding to residue 228 in SEQ ID NO: 27; M at a residue corresponding to residue 248 in SEQ ID NO: 27; C at a residue corresponding to residue 256 in SEQ ID NO: 27; Q or C at a residue corresponding to residue 293 in SEQ ID NO: 27; K or N at a residue corresponding to residue 296 in SEQ ID NO: 27; R, Q, or K at a residue corresponding to residue 297 in SEQ ID NO: 27; C or D at a residue corresponding to residue 300 in SEQ ID NO: 27; T or S at a residue corresponding to residue 302 in SEQ ID NO: 27; C at a residue corresponding to residue 305 in SEQ ID NO: 27; F at a residue corresponding to residue 319 in SEQ ID NO: 27; and/or M at a residue corresponding to residue 330 in SEQ ID NO: 27.

In some embodiments, a LeuDH enzyme comprises: V at a residue corresponding to residue 13 in SEQ ID NO: 27; W at a residue corresponding to residue 16 in SEQ ID NO: 27; Q at a residue corresponding to residue 42 in SEQ ID NO: 27; T, Y, F, E, or W at a residue corresponding to residue 43 in SEQ ID NO: 27; I, H, K, or Y at a residue corresponding to residue 44 in SEQ ID NO: 27; T, E, A, S, or K at a residue corresponding to residue 67 in SEQ ID NO: 27; K at a residue corresponding to residue 71 in SEQ ID NO: 27; S at a residue corresponding to residue 73 in SEQ ID NO: 27; R, H, Y, S, K, or W at a residue corresponding to residue 76 in SEQ ID NO: 27; Y at a residue corresponding to residue 92 in SEQ ID NO: 27; H at a residue corresponding to residue 93 in SEQ ID NO: 27; G at a residue corresponding to residue 95 in SEQ ID NO: 27; G at a residue corresponding to residue 100 in SEQ ID NO: 27; C at a residue corresponding to residue 105 in SEQ ID NO: 27; G at a residue corresponding to residue 111 in SEQ ID NO: 27; M at a residue corresponding to residue 113 in SEQ ID NO: 27; N, or V at a residue corresponding to residue 115 in SEQ ID NO: 27; R, N, or W at a residue corresponding to residue 116 in SEQ ID NO: 27; A at a residue corresponding to residue 120 in SEQ ID NO: 27; D at a residue corresponding to residue 122 in SEQ ID NO: 27; E at a residue corresponding to residue 136 in SEQ ID NO: 27; D at a residue corresponding to residue 140 in SEQ ID NO: 27; M at a residue corresponding to residue 141 in SEQ ID NO: 27; S at a residue corresponding to residue 160 in SEQ ID NO: 27; F at a residue corresponding to residue 185 in SEQ ID NO: 27; N at a residue corresponding to residue 196 in SEQ ID NO: 27; Y at a residue corresponding to residue 228 in SEQ ID NO: 27; M at a residue corresponding to residue 248 in SEQ ID NO: 27; C at a residue corresponding to residue 256 in SEQ ID NO: 27; Q or C at a residue corresponding to residue 293 in SEQ ID NO: 27; K or N at a residue corresponding to residue 296 in SEQ ID NO: 27; R, Q, or K at a residue corresponding to residue 297 in SEQ ID NO: 27; C or D at a residue corresponding to residue 300 in SEQ ID NO: 27; T or S at a residue corresponding to residue 302 in SEQ ID NO: 27; C at a residue corresponding to residue 305 in SEQ ID NO: 27; F at a residue corresponding to residue 319 in SEQ ID NO: 27; and M at a residue corresponding to residue 330 in SEQ ID NO: 27.

In some embodiments, a LeuDH enzyme comprises at least 1, at least 2, at least 3, at least 4, at least 5, at least 6, at least 7, at least 8, at least 9, at least 10, at least 11, at least 12, at least 13, at least, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 60, at least 70, at least 80, at least 90, or at least 100 amino acid substitutions, deletions, insertions, or additions relative to SEQ ID NO: 27, any one of SEQ ID NO: 2, 4, 6, 8, 10, 12, or 257-475, a LeuDH enzyme in Table 3 or Table 4, or a LeuDH enzyme otherwise described in this disclosure.

In some embodiments, a LeuDH enzyme comprises an amino acid substitution at one or more residues relative to SEQ ID NO: 27. In some embodiments, a LeuDH enzyme comprises an amino acid substitution at a residue corresponding to position 42 in SEQ ID NO: 27, at a residue corresponding to position 43 in SEQ ID NO: 27, at a residue corresponding to position 44 in SEQ ID NO: 27, at a residue corresponding to position 67 in SEQ ID NO: 27, at a residue corresponding to position 71 in SEQ ID NO: 27, at a residue corresponding to position 76 in SEQ ID NO: 27, at a residue corresponding to position 78 in SEQ ID NO: 27, at a residue corresponding to position 113 in SEQ ID NO: 27, at a residue corresponding to position 115 in SEQ ID NO: 27, at a residue corresponding to position 116 in SEQ ID NO: 27, at a residue corresponding to position 136 in SEQ ID NO: 27, at a residue corresponding to position 293 in SEQ ID NO: 27, at a residue corresponding to position 296 in SEQ ID NO: 27, at a residue corresponding to position 297 in SEQ ID NO: 27, and/or at a residue corresponding to position 300 in SEQ ID NO: 27. In some embodiments, a LeuDH enzyme comprises: A, Q, or T at a residue corresponding to position 42 in SEQ ID NO: 27; E, F, T, W, or Y at a residue corresponding to position 43 in SEQ ID NO: 27; H, I, K, or Y at a residue corresponding to position 44 in SEQ ID NO: 27; A, E, K, Q, S, or T at a residue corresponding to position 67 in SEQ ID NO: 27; C, D, H, K, M, or T at a residue corresponding to position 71 in SEQ ID NO: 27; E, F, H, I, K, M, R, S, T, W, or Y at a residue corresponding to position 76 in SEQ ID NO: 27; C, F, H, K, Q, V, or Y at a residue corresponding to position 78 in SEQ ID NO: 27; F, M, Q, V, W, or Y at a residue corresponding to position 113 in SEQ ID NO: 27; N, Q, S, T, or V at a residue corresponding to position 115 in SEQ ID NO: 27; A, L, M, N, R, S, V, or W at a residue corresponding to position 116 in SEQ ID NO: 27; E, F, L, R, S, or Y at a residue corresponding to position 136 in SEQ ID NO: 27; A, C, Q, S, or T at a residue corresponding to position 293 in SEQ ID NO: 27; A, C, E, I, K, L, N, S, or T at a residue corresponding to position 296 in SEQ ID NO: 27; C, D, E, F, H, K, L, M, N, Q, R, T, W, or Y at a residue corresponding to position 297 in SEQ ID NO: 27; and/or A, C, D, F, H, K, M, N, Q, R, S, T, W, or Y at a residue corresponding to position 300 in SEQ ID NO: 27.

In some embodiments, relative to SEQ ID NO: 27, a LeuDH enzyme comprises an amino acid substitution at amino acid residue: 42, 43, 44, 67, 71, 76, 78, 113, 115, 116, 136, 293, 296, 297 and/or 300. In some embodiments, a LeuDH enzyme comprises A, Q, or T at residue 42; E, F, T, W, or Y at residue 43; H, I, K, or Y at residue 44; A, E, K, Q, S, or T at residue 67; C, D, H, K, M, or T at residue 71; E, F, H, I, K, M, R, S, T, W, or Y at residue 76; C, F, H, K, Q, V, or Y at residue 78; F, M, Q, V, W, or Y at residue 113; N, Q, S, T, or V at residue 115; A, L, M, N, R, S, V, or W at residue 116; E, F, L, R, S, or Y at residue 136; A, C, Q, S, or T at residue 293; A, C, E, I, K, L, N, S, or T at residue 296; C, D, E, F, H, K, L, M, N, Q, R, T, W, or Y at residue 297; and/or A, C, D, F, H, K, M, N, Q, R, S, T, W, or Y at residue 300.

### Ketoisovalerate Decarboxylase (KivD)

As used in this disclosure “ketoisovalerate decarboxylase (KivD)” refers to an enzyme that catalyzes the decarboxylation of alpha-keto acids derived from amino acid transamination into aldehydes. A KivD may use ketoisocaproate as a substrate. In some embodiments, KivD produces isovaleraldehyde from ketoisocaproate.

In some embodiments, a host cell comprises a KivD enzyme and/or a heterologous polynucleotide encoding such an enzyme. In some embodiments, a host cell comprises a heterologous polynucleotide encoding a KivD enzyme comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to any one of SEQ ID NO: 14, 16, 18, or 533-588, a KivD enzyme in Table 3 or Table 5, or a KivD enzyme otherwise described in this disclosure. In some embodiments, a host cell comprises a heterologous polynucleotide that is at least 90% (e.g., at least 90%, at least 91%, at least 92%, at least 93%, at least 94%, at least 95%, at least 96%, at least 97%, at least 98%, at least 99%, or 100%) identical to any one of SEQ ID NO: 13, 15, 17 or 477-532, a polynucleotide encoding a KivD enzyme in Table 3 or Table 5, or a polynucleotide encoding a KivD enzyme otherwise described in this disclosure.

In some embodiments, a host cell comprises KivD from Lactococcus lactis. In other embodiments, a host cell does not comprise KivD from Lactococcus lactis.

KivD from Lactococcus lactis can comprise the amino acid sequence of UniProtKB—Q684J7 (SEQ ID NO: 29):

(SEQ ID NO: 29)
MYTVGDYLLDRLHELGIEEIFGVPGDYNLQFLDQIISHKDMKWVGNANEL
NASYMADGYARTKKAAAFLTTFGVGELSAVNGLAGSYAENLPVVEIVGSP
TSKVQNEGKFVHHTLADGDFKHFMKMHEPVTAARTLLTAENATVEIDRVL
SALLKERKPVYINLPVDVAAAKAEKPSLPLKKENSTSNTSDQEILNKIQE
SLKNAKKPIVITGHEIISFGLEKTVTQFISKTKLPITTLNFGKSSVDEAL
PSFLGIYNGTLSEPNLKEFVESADFILMLGVKLTDSSTGAFTHHLNENKM
ISLNIDEGKIFNERIQNFDFESLISSLLDLSEIEYKGKYIDKKQEDFVPS
NALLSQDRLWQAVENLTQSNETIVAEQGTSFFGASSIFLKSKSHFIGQPL
WGSIGYTFPAALGSQIADKESRHLLFIGDGSLQLTVQELGLAIREKINPI
CFIINNDGYTVEREIHGPNQSYNDIPMWNYSKLPESFGATEDRVVSKIVR
TENEFVSVMKEAQADPNRMYWIELILAKEGAPKVLKKMGKLFAEQNKS

In some embodiments, the amino acid sequence of SEQ ID NO: 29 is encoded by the nucleic acid sequence:

(SEQ ID NO: 30)
ATGTACACAGTCGGTGATTATCTTTTAGACCGACTGCACGAACTCGGAAT
CGAGGAAATTTTTGGCGTGCCCGGGGATTATAACTTGCAGTTCCTGGACC
AAATAATTTCCCATAAGGATATGAAATGGGTAGGCAATGCTAACGAACTG
AATGCGTCTTACATGGCCGATGGTTATGCACGGACCAAAAAAGCGGCAGC
CTTTCTGACGACTTTCGGCGTTGGTGAGTTAAGCGCGGTGAACGGCCTGG
CGGGGTCATACGCCGAAAATCTACCAGTTGTCGAAATCGTGGGCTCGCCG
ACCAGCAAAGTTCAGAACGAGGGTAAGTTTGTGCATCACACCCTTGCTGA
CGGAGATTTTAAACATTTCATGAAAATGCACGAACCTGTAACGGCAGCGC
GCACACTGTTGACTGCGGAGAACGCCACCGTCGAAATTGATCGCGTCCTG
AGTGCTCTTCTGAAGGAACGTAAACCGGTGTATATCAATCTCCCGGTTGA
CGTGGCGGCAGCTAAAGCCGAAAAACCGAGTTTGCCCTTAAAGAAAGAGA
ATAGCACGTCTAACACGTCTGACCAAGAAATTCTGAACAAAATTCAGGAA
TCCCTCAAAAATGCGAAAAAACCTATCGTCATCACCGGTCATGAAATAAT
TTCATTTGGACTGGAGAAAACCGTTACACAGTTCATCTCAAAGACGAAAC
TGCCAATTACCACCCTAAATTTTGGCAAATCGTCCGTAGACGAAGCCCTG
CCGAGCTTCTTGGGGATCTATAACGGCACTTTAAGCGAACCGAATTTAAA
GGAATTTGTGGAGAGCGCCGATTTCATTCTCATGCTGGGTGTTAAGCTGA
CAGATTCCAGTACGGGCGCGTTCACTCATCACCTGAACGAGAACAAAATG
ATCTCGTTGAACATTGATGAAGGAAAAATATTTAATGAACGTATTCAAAA
CTTCGATTTTGAATCGCTGATTTCTTCCCTACTGGACCTCAGCGAGATCG
AATACAAAGGTAAATATATTGATAAAAAACAGGAAGACTTTGTGCCGAGT
AACGCACTGTTGTCTCAGGATCGCCTGTGGCAAGCTGTGGAAAATCTGAC
CCAGAGTAACGAAACGATTGTCGCGGAACAGGGGACCTCTTTCTTTGGTG
CTTCGTCAATCTTTTTAAAGTCAAAATCACATTTTATTGGCCAACCACTT
TGGGGTAGTATCGGCTACACTTTCCCTGCGGCACTGGGTAGTCAGATTGC
CGATAAAGAGTCGCGTCACCTTTTGTTTATTGGGGATGGCTCGCTACAAT
TGACCGTTCAGGAGTTAGGTCTTGCTATACGCGAAAAAATCAATCCGATC
TGTTTCATTATCAATAATGACGGCTATACCGTGGAGCGCGAAATCCATGG
TCCGAATCAGAGCTATAACGATATACCGATGTGGAATTACAGCAAACTCC
CCGAGAGCTTTGGCGCAACAGAAGATAGGGTTGTCTCCAAGATCGTGCGT
ACGGAAAACGAATTTGTAAGTGTAATGAAAGAAGCGCAAGCGGACCCTAA
TCGAATGTACTGGATTGAACTTATTCTGGCAAAAGAAGGGGCCCCTAAAG
TCCTCAAGAAAATGGGGAAGTTGTTCGCCGAACAAAACAAAAGCTGA

In some embodiments, a host cell that expresses a heterologous polynucleotide encoding a KivD enzyme may increase conversion of ketoisocaproate to isovaleraldehyde by 0.5-fold, 1-fold, 1.5-fold, 2-fold, 2.5-fold, 3-fold, 3.5-fold, 4-fold, 4.5-fold, 5-fold, 5.5-fold, or 6-fold more (e.g., 2-fold to 6-fold more) relative to a control. In some embodiments, a control is a host cell that expresses a heterologous polynucleotide encoding SEQ ID NO: 29. In some embodiments, the control is an E. coli Nissle strain SYN1980 ΔleuE, ΔilvC, lacZ:tetR-Ptet-livKHMGF, tetR-Ptet-leuDH(Bc)-kivD-adh2-brnQ-rrnB ter (pSC101), such as is described in U.S. Patent Application Publication No. US20170232043.

In some embodiments, a KivD enzyme comprises a sequence that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or is 100% identical to SEQ ID NO: 29, any one of SEQ ID NO: 14, 16, 18, or 533-588, any one of SEQ ID NO: 13, 15, 17 or 477-532, an amino acid or polynucleotide sequence encoding a KivD enzyme in Table 3 or Table 5, or a KivD enzyme otherwise described in this disclosure.

In some embodiments, a KivD enzyme comprises at least 1, at least 2, at least 3, at least 4, at least 5, at least 6, at least 7, at least 8, at least 9, at least 10, at least 11, at least 12, at least 13, at least, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 60, at least 70, at least 80, at least 90, or at least 100 amino acid substitutions, deletions, insertions, or additions relative to SEQ ID NO: 29, any one of SEQ ID NO: 14, 16, 18, or 533-588, a KivD enzyme in Table 3 or Table 5, or a KivD enzyme otherwise described in this disclosure.

In some embodiments, a KivD enzyme comprises: Y at a residue corresponding to residue 33 in SEQ ID NO: 29; Q at a residue corresponding to residue 44 in SEQ ID NO: 29; M at a residue corresponding to residue 117 in SEQ ID NO: 29; I at a residue corresponding to residue 129 in SEQ ID NO: 29; W at a residue corresponding to residue 185 in SEQ ID NO: 29; I at a residue corresponding to residue 190 in SEQ ID NO: 29; I at a residue corresponding to residue 225 in SEQ ID NO: 29; Y at a residue corresponding to residue 227 in SEQ ID NO: 29; L at a residue corresponding to residue 311 in SEQ ID NO: 29; G at a residue corresponding to residue 312 in SEQ ID NO: 29; T at a residue corresponding to residue 313 in SEQ ID NO: 29; P at a residue corresponding to residue 328 in SEQ ID NO: 29; W at a residue corresponding to residue 341 in SEQ ID NO: 29; H at a residue corresponding to residue 345 in SEQ ID NO: 29; C at a residue corresponding to residue 347 in SEQ ID NO: 29; R at a residue corresponding to residue 420 in SEQ ID NO: 29; D at a residue corresponding to residue 494 in SEQ ID NO: 29; C at a residue corresponding to residue 508 in SEQ ID NO: 29; and/or F at a residue corresponding to residue 550 in SEQ ID NO: 29.

In some embodiments, a KivD enzyme comprises: Y at a residue corresponding to residue 33 in SEQ ID NO: 29; Q at a residue corresponding to residue 44 in SEQ ID NO: 29; M at a residue corresponding to residue 117 in SEQ ID NO: 29; I at a residue corresponding to residue 129 in SEQ ID NO: 29; W at a residue corresponding to residue 185 in SEQ ID NO: 29; I at a residue corresponding to residue 190 in SEQ ID NO: 29; I at a residue corresponding to residue 225 in SEQ ID NO: 29; Y at a residue corresponding to residue 227 in SEQ ID NO: 29; L at a residue corresponding to residue 311 in SEQ ID NO: 29; G at a residue corresponding to residue 312 in SEQ ID NO: 29; T at a residue corresponding to residue 313 in SEQ ID NO: 29; P at a residue corresponding to residue 328 in SEQ ID NO: 29; W at a residue corresponding to residue 341 in SEQ ID NO: 29; H at a residue corresponding to residue 345 in SEQ ID NO: 29; C at a residue corresponding to residue 347 in SEQ ID NO: 29; R at a residue corresponding to residue 420 in SEQ ID NO: 29; D at a residue corresponding to residue 494 in SEQ ID NO: 29; C at a residue corresponding to residue 508 in SEQ ID NO: 29; and F at a residue corresponding to residue 550 in SEQ ID NO: 29.

### Alcohol Dehydrogenase (Adh)

As used in this disclosure “alcohol dehydrogenase (Adh)” refers to an enzyme that catalyzes the conversion of ethanol to acetaldehyde. An Adh may use isovaleraldehyde as a substrate. In some embodiments, Adh produces isopentanol from isovaleraldehyde.

In some embodiments, a host cell comprises an Adh enzyme and/or a heterologous polynucleotide encoding such an enzyme. In some embodiments, a host cell comprises a heterologous polynucleotide encoding an Adh enzyme comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to any one of SEQ ID NO: 20, 22, 24, or 645-700, an Adh enzyme in Table 3 or Table 6, or an Adh enzyme otherwise described in this disclosure. In some embodiments, a host cell comprises a heterologous polynucleotide that is at least 90% (e.g., at least 90%, at least 91%, at least 92%, at least 93%, at least 94%, at least 95%, at least 96%, at least 97%, at least 98%, at least 99%, or 100%) identical to any one of SEQ ID NO: 19, 21, 23 or 589-644, a polynucleotide encoding a Adh enzyme in Table 3 or Table 6, or an Adh enzyme otherwise described in this disclosure.

In some embodiments, a host cell comprises Adh from Saccharomyces cerevisiae. In other embodiments, a host cell does not comprise Adh from Saccharomyces cerevisiae.

Adh from Saccharomyces cerevisiae can comprises the amino acid sequence of UniProtKB—P00331 (SEQ ID NO: 31):

(SEQ ID NO: 31)
MSIPETQKAIIFYESNGKLEHKDIPVPKPKPNELLINVKYSGVCHTDLHA
WHGDWPLPTKLPLVGGHEGAGVVVGMGENVKGWKIGDYAGIKWLNGSCMA
CEYCELGNESNCPHADLSGYTHDGSFQEYATADAVQAAHIPQGTDLAEVA
PILCAGITVYKALKSANLRAGHWAAISGAAGGLGSLAVQYAKAMGYRVLG
IDGGPGKEELFTSLGGEVEIDFTKEKDIVSAVVKATNGGAHGIINVSVSE
AAIEASTRYCRANGTVVLVGLPAGAKCSSDVFNHVVKSISIVGSYVGNRA
DTREALDFFARGLVKSPIKVVGLSSLPEIYEKMEKGQIAGRYVVDTSK

In some embodiments, the amino acid sequence of SEQ ID NO: 3 μs encoded by the nucleic acid sequence:

(SEQ ID NO: 32)
ATGTCGATCCCAGAAACTCAGAAGGCTATTATATTTTATGAGTCAAACGG
CAAACTCGAACATAAAGACATTCCCGTGCCTAAACCGAAACCGAATGAAC
TTCTGATTAACGTAAAGTACAGCGGAGTCTGCCACACGGATTTGCATGCC
TGGCACGGGGATTGGCCGTTACCGACCAAACTGCCTCTGGTGGGTGGTCA
TGAGGGCGCGGGCGTTGTTGTGGGTATGGGAGAAAATGTCAAAGGCTGGA
AAATCGGCGACTATGCAGGGATCAAGTGGCTGAACGGGTCTTGTATGGCG
TGCGAGTACTGTGAATTAGGTAATGAATCCAACTGCCCACACGCAGATCT
GAGTGGTTATACCCATGACGGCAGCTTCCAAGAATACGCCACAGCGGATG
CCGTGCAGGCAGCTCACATTCCGCAAGGAACTGATCTTGCGGAAGTAGCC
CCAATTCTGTGCGCGGGCATCACGGTATATAAAGCTCTCAAAAGTGCAAA
CTTGCGCGCCGGTCATTGGGCTGCGATTTCGGGTGCCGCGGGCGGGCTGG
GATCATTAGCTGTTCAGTACGCGAAGGCAATGGGTTATCGAGTTCTGGGC
ATCGACGGCGGGCCCGGTAAAGAAGAGCTATTTACCAGCCTCGGCGGTGA
GGTCTTCATCGATTTTACCAAAGAAAAAGATATCGTGTCCGCAGTCGTGA
AAGCAACCAATGGCGGCGCTCACGGAATTATAAATGTGTCTGTATCAGAA
GCGGCGATTGAAGCCAGCACGCGTTATTGTCGCGCGAACGGCACAGTGGT
TCTGGTAGGCCTGCCCGCCGGTGCGAAATGTAGCTCGGACGTGTTCAATC
ATGTGGTGAAGAGTATTTCCATTGTTGGATCTTACGTAGGGAACCGTGCG
GATACGCGGGAGGCACTGGATTTTTTTGCAAGGGGCTTGGTTAAAAGCCC
GATCAAAGTCGTGGGTCTGTCGTCTCTACCTGAAATATATGAGAAAATGG
AAAAGGGACAGATCGCCGGACGCTACGTCGTCGACACCTCAAAGTGA

In some embodiments, a host cell that expresses a heterologous polynucleotide encoding an Adh enzyme may increase conversion of isovaleraldehyde to isopentanol by 0.5-fold, 1-fold, 1.5-fold, 2-fold, 2.5-fold, 3-fold, 3.5-fold, 4-fold, 4.5-fold, 5-fold, 5.5-fold, or 6-fold more (e.g., 2-fold to 6-fold more) relative to a control. In some embodiments, a control is a host cell that expresses a heterologous polynucleotide encoding SEQ ID NO: 31. In some embodiments, a control is a host cell that expresses a heterologous polynucleotide encoding SEQ ID NO: 31. In some embodiments, the control is an E. coli Nissle strain SYN1980 ΔleuE, ΔilvC, lacZ:tetR-Ptet-livKHMGF, tetR-Ptet-leuDH(Bc)-kivD-adh2-brnQ-rrnB ter (pSC101), such as is described in U.S. Patent Application Publication No. US20170232043.

In some embodiments, an Adh comprises a sequence that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or is 100% identical to SEQ ID NO: 31, any one of SEQ ID NO: 20, 22, 24, or 645-700, any one of SEQ ID NO: 19, 21, 23 or 589-644, an amino acid or polynucleotide sequence encoding a Adh enzyme in Table 3 or Table 6, or an Adh enzyme otherwise disclosed in this disclosure.

In some embodiments, an Adh comprises at least 1, at least 2, at least 3, at least 4, at least 5, at least 6, at least 7, at least 8, at least 9, at least 10, at least 11, at least 12, at least 13, at least, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 60, at least 70, at least 80, at least 90, or at least 100 amino acid substitutions, deletions, insertions, or additions relative to SEQ ID NO: 31, any one of SEQ ID NO: 20, 22, 24, or 645-700, an Adh enzyme in Table 3 or Table 6, or an Adh enzyme otherwise disclosed in this disclosure.

In some embodiments, an Adh comprises P at a residue corresponding to residue 9 in SEQ ID NO: 31; G at a residue corresponding to residue 16 in SEQ ID NO: 31; Q at a residue corresponding to residue 23 in SEQ ID NO: 31; R at a residue corresponding to residue 28 in SEQ ID NO: 31; A at a residue corresponding to residue 30 in SEQ ID NO: 31; K at a residue corresponding to residue 93 in SEQ ID NO: 31; L at a residue corresponding to residue 98 in SEQ ID NO: 31; R at a residue corresponding to residue 99 in SEQ ID NO: 31; P at a residue corresponding to residue 114 in SEQ ID NO: 31; K at a residue corresponding to residue 115 in SEQ ID NO: 31; Y at a residue corresponding to residue 119 in SEQ ID NO: 31; Y at a residue corresponding to residue 194 in SEQ ID NO: 31; P at a residue corresponding to residue 242 in SEQ ID NO: 31; K at a residue corresponding to residue 249 in SEQ ID NO: 31; E at a residue corresponding to residue 255 in SEQ ID NO: 31; D at a residue corresponding to residue 260 in SEQ ID NO: 31; H at a residue corresponding to residue 269 in SEQ ID NO: 31; Q at a residue corresponding to residue 281 in SEQ ID NO: 31; L at a residue corresponding to residue 325 in SEQ ID NO: 31; M at a residue corresponding to residue 333 in SEQ ID NO: 31; P at a residue corresponding to residue 334 in SEQ ID NO: 31; and/or Q at a residue corresponding to residue 348 in SEQ ID NO: 31.

In some embodiments, an Adh comprises P at a residue corresponding to residue 9 in SEQ ID NO: 31; G at a residue corresponding to residue 16 in SEQ ID NO: 31; Q at a residue corresponding to residue 23 in SEQ ID NO: 31; R at a residue corresponding to residue 28 in SEQ ID NO: 31; A at a residue corresponding to residue 30 in SEQ ID NO: 31; K at a residue corresponding to residue 93 in SEQ ID NO: 31; L at a residue corresponding to residue 98 in SEQ ID NO: 31; R at a residue corresponding to residue 99 in SEQ ID NO: 31; P at a residue corresponding to residue 114 in SEQ ID NO: 31; K at a residue corresponding to residue 115 in SEQ ID NO: 31; Y at a residue corresponding to residue 119 in SEQ ID NO: 31; Y at a residue corresponding to residue 194 in SEQ ID NO: 31; P at a residue corresponding to residue 242 in SEQ ID NO: 31; K at a residue corresponding to residue 249 in SEQ ID NO: 31; E at a residue corresponding to residue 255 in SEQ ID NO: 31; D at a residue corresponding to residue 260 in SEQ ID NO: 31; H at a residue corresponding to residue 269 in SEQ ID NO: 31; Q at a residue corresponding to residue 281 in SEQ ID NO: 31; L at a residue corresponding to residue 325 in SEQ ID NO: 31; M at a residue corresponding to residue 333 in SEQ ID NO: 31; P at a residue corresponding to residue 334 in SEQ ID NO: 31; and Q at a residue corresponding to residue 348 in SEQ ID NO: 31.

### Branched-Chain Amino Acid Transport System 2 Carrier Protein (BrnQ)

As used in this disclosure “Branched-chain amino acid transport system 2 carrier protein (BrnQ)” refers to a component of the LIV-II transport system for branched-chain amino acids. BrnQ may be used to transport a branched-chain amino acid, e.g., leucine, into a cell such as a host cell.

In some embodiments, a host cell comprises a BrnQ protein and/or a heterologous polynucleotide encoding such a protein. In some embodiments, a host cell comprises a heterologous polynucleotide encoding a BrnQ protein comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to a BrnQ protein as described in this application, e.g., SEQ ID NO: 35. In some embodiments, the BrnQ protein comprises the amino acid sequence set forth in UniProtKB—B7MD59.

UniProtKB—B7MD59 has the amino acid sequence:

(SEQ ID NO: 35)
MTHQLRSRDIIALGFMTFALFVGAGNIIFPPMVGLQAGEHVWTAAFGFLI
TAVGLPVLTVVALAKVGGGVDSLSTPIGKVAGVLLATVCYLAVGPLFATP
RTATVSFEVGIAPLTGDSALPLFIYSLVYFAIVILVSLYPGKLLDTVGNF
LAPLKIIALVILSVAAIIWPAGSISTATEAYQNAAFSNGFVNGYLTMDTL
GAMVFGIVIVNAARSRGVTEARLLTRYTVWAGLMAGVGLTLLYLALFRLG
SDSASLVDQSANGAAILHAYVQHTFGGGGSFLLAALIFIACLVTAVGLTC
ACAEFFAQYVPLSYRTLVFILGGFSMVVSNLGLSQLIQISVPVLTAIYPP
CIALVVLSFTRSWWHNSSRVIAPPMFISLLFGILDGIKASAFSDILPSWA
QRLPLAEQGLAWLMPTVVMVVLAIIWDRAAGRQVTSSAH

In some embodiments, SEQ ID NO: 35 is encoded by the nucleic acid sequence:

(SEQ ID NO: 36)
ATGACCCATCAATTAAGATCGCGCGATATCATCGCTCTGGGCTTTATGAC
ATTTGCGTTGTTCGTCGGCGCAGGTAACATTATTTTCCCTCCAATGGTCG
GCTTGCAGGCAGGCGAACACGTCTGGACTGCGGCATTCGGCTTCCTCATT
ACTGCCGTTGGCCTACCGGTATTAACGGTAGTGGCGCTGGCAAAAGTTGG
CGGCGGTGTTGACAGTCTCAGCACGCCAATTGGTAAAGTCGCTGGCGTAC
TGCTGGCAACAGTTTGTTACCTGGCGGTGGGGCCGCTTTTTGCTACGCCG
CGTACAGCTACCGTTTCTTTTGAAGTGGGCATTGCGCCGCTGACGGGTGA
TTCCGCGCTGCCGCTGTTTATTTACAGCCTGGTCTATTTCGCTATCGTTA
TTCTGGTTTCGCTCTATCCGGGCAAGCTGCTGGATACCGTGGGCAACTTC
CTTGCGCCGCTGAAAATTATCGCGCTGGTCATCCTGTCTGTTGCCGCAAT
TATCTGGCCGGCGGGTTCTATCAGTACGGCGACTGAGGCTTATCAAAACG
CTGCGTTTTCTAACGGCTTCGTCAACGGCTATCTGACCATGGATACGCTG
GGCGCAATGGTGTTTGGTATCGTTATTGTTAACGCGGCGCGTTCTCGTGG
CGTTACCGAAGCGCGTCTGCTGACCCGTTATACCGTCTGGGCTGGCCTGA
TGGCGGGTGTTGGTCTGACTCTGCTGTACCTGGCGCTGTTCCGTCTGGGT
TCAGACAGCGCGTCGCTGGTCGATCAGTCTGCAAACGGTGCGGCGATCCT
GCATGCTTACGTTCAGCATACCTTTGGCGGCGGCGGTAGCTTCCTGCTGG
CGGCGTTAATCTTCATCGCCTGCCTGGTCACGGCGGTTGGCCTGACCTGT
GCTTGTGCAGAATTCTTCGCCCAGTACGTACCGCTCTCTTATCGTACGCT
GGTGTTTATCCTCGGCGGCTTCTCGATGGTGGTGTCTAACCTCGGCTTGA
GCCAGCTGATTCAGATCTCTGTACCGGTGCTGACCGCCATTTATCCGCCG
TGTATCGCACTGGTTGTATTAAGTTTTACACGCTCATGGTGGCATAATTC
GTCCCGCGTGATTGCTCCGCCGATGTTTATCAGCCTGCTTTTTGGTATTC
TCGACGGGATCAAGGCATCTGCATTCAGCGATATCTTACCGTCCTGGGCG
CAGCGTTTACCGCTGGCCGAACAAGGTCTGGCGTGGTTAATGCCAACAGT
GGTGATGGTGGTTCTGGCCATTATCTGGGATCGTGCGGCAGGTCGTCAGG
TGACCTCCAGCGCTCACTAA

### Variants

Variants of enzymes and proteins described in this disclosure (e.g., LeuDH, KivD, or Adh and including variants to nucleic acid and amino acid sequences) are also encompassed by the present disclosure. A variant may share at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity with a reference sequence, including all values in between.

Unless otherwise noted, the term “sequence identity,” as known in the art, refers to a relationship between the sequences of two polypeptides or polynucleotides, as determined by sequence comparison (alignment). In some embodiments, sequence identity is determined across the entire length of a sequence (e.g., LeuDH, KivD, or Adh sequence). In some embodiments, sequence identity is determined over a region (e.g., a stretch of amino acids or nucleic acids, e.g., the sequence spanning an active site) of a sequence (e.g., LeuDH, KivD, or Adh sequence).

Identity can also refer to the degree of sequence relatedness between two sequences as determined by the number of matches between strings of two or more residues (e.g., nucleic acid or amino acid residues). Identity measures the percent of identical matches between two or more sequences with gap alignments (if any) addressed by a particular mathematical model or computer program (e.g., algorithms).

Identity of related polypeptides or nucleic acid sequences can be readily calculated by any of the methods known to one of ordinary skill in the art. The “percent identity” of two sequences (e.g., nucleic acid or amino acid sequences) may, for example, be determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST® and XBLAST® programs (version 2.0) of Altschul et al., J. Mol. Biol. 215:403-10, 1990. BLAST® protein searches can be performed, for example, with the XBLAST program, score=50, wordlength=3 to obtain amino acid sequences homologous to the proteins described in this application. Where gaps exist between two sequences, Gapped BLAST® can be utilized, for example, as described in Altschul et al., Nucleic Acids Res. 25(17):3389-3402, 1997. When utilizing BLAST® and Gapped BLAST® programs, the default parameters of the respective programs (e.g., XBLAST® and NBLAST®) can be used, or the parameters can be adjusted appropriately as would be understood by one of ordinary skill in the art.

Another local alignment technique which may be used, for example, is based on the Smith-Waterman algorithm (Smith, T. F. & Waterman, M. S. (1981) “Identification of common molecular subsequences.” J. Mol. Biol. 147:195-197). A general global alignment technique which may be used, for example, is the Needleman—Wunsch algorithm (Needleman, S. B. & Wunsch, C. D. (1970) “A general method applicable to the search for similarities in the amino acid sequences of two proteins.” J. Mol. Biol. 48:443-453), which is based on dynamic programming.

More recently, a Fast Optimal Global Sequence Alignment Algorithm (FOGSAA) was developed that purportedly produces global alignment of nucleic acid and amino acid sequences faster than other optimal global alignment methods, including the Needleman-Wunsch algorithm. In some embodiments, the identity of two polypeptides is determined by aligning the two amino acid sequences, calculating the number of identical amino acids, and dividing by the length of one of the amino acid sequences. In some embodiments, the identity of two nucleic acids is determined by aligning the two nucleotide sequences and calculating the number of identical nucleotide and dividing by the length of one of the nucleic acids.

For multiple sequence alignments, computer programs including Clustal Omega (Sievers et al., Mol Syst Biol. 2011 Oct. 11; 7:539) may be used.

In preferred embodiments, a sequence, including a nucleic acid or amino acid sequence, is found to have a specified percent identity to a reference sequence, such as a sequence disclosed in this application and/or recited in the claims when sequence identity is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993 (e.g., BLAST®, NBLAST®, XBLAST® or Gapped BLAST® programs, using default parameters of the respective programs).

In some embodiments, a sequence, including a nucleic acid or amino acid sequence, is found to have a specified percent identity to a reference sequence, such as a sequence disclosed in this application and/or recited in the claims when sequence identity is determined using the Smith-Waterman algorithm (Smith, T. F. & Waterman, M. S. (1981) “Identification of common molecular subsequences.” J. Mol. Biol. 147:195-197) or the Needleman-Wunsch algorithm (Needleman, S. B. & Wunsch, C. D. (1970) “A general method applicable to the search for similarities in the amino acid sequences of two proteins.” J. Mol. Biol. 48:443-453) using default parameters.

In some embodiments, a sequence, including a nucleic acid or amino acid sequence, is found to have a specified percent identity to a reference sequence, such as a sequence disclosed in this application and/or recited in the claims when sequence identity is determined using a Fast Optimal Global Sequence Alignment Algorithm (FOGSAA) using default parameters.

In some embodiments, a sequence, including a nucleic acid or amino acid sequence, is found to have a specified percent identity to a reference sequence, such as a sequence disclosed in this application and/or recited in the claims when sequence identity is determined using Clustal Omega (Sievers et al., Mol Syst Biol. 2011 Oct. 11; 7:539) using default parameters.

As used in this disclosure, a residue (such as a nucleic acid residue or an amino acid residue) in sequence “X” is referred to as corresponding to a position or residue (such as a nucleic acid residue or an amino acid residue) “Z” in a different sequence “Y” when the residue in sequence “X” is at the counterpart position of “Z” in sequence “Y” when sequences X and Y are aligned using amino acid sequence alignment tools known in the art, such as, for example, Clustal Omega or BLAST®.

As used in this disclosure, variant sequences may be homologous sequences. As used in this disclosure, homologous sequences are sequences (e.g., nucleic acid or amino acid sequences) that share a certain percent identity (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% percent identity, including all values in between). Homologous sequences include but are not limited to paralogous or orthologous sequences. Paralogous sequences arise from duplication of a gene within a genome of a species, while orthologous sequences diverge after a speciation event.

In some embodiments, a polypeptide variant (e.g., LeuDH, KivD, or Adh enzyme variant) comprises a domain that shares a secondary structure (e.g., alpha helix, beta sheet) with a reference polypeptide (e.g., a reference LeuDH, KivD, or Adh enzyme). In some embodiments, a polypeptide variant (e.g., LeuDH, KivD, or Adh enzyme variant) shares a tertiary structure with a reference polypeptide (e.g., a reference LeuDH, KivD, or Adh enzyme). As a non-limiting example, a variant polypeptide (e.g., LeuDH, KivD, or Adh enzyme variant) may have low primary sequence identity (e.g., less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, or less than 5% sequence identity) compared to a reference polypeptide, but share one or more secondary structures (e.g., including but not limited to loops, alpha helices, or beta sheets), or have the same tertiary structure as a reference polypeptide. For example, a loop may be located between a beta sheet and an alpha helix, between two alpha helices, or between two beta sheets. Homology modeling may be used to compare two or more tertiary structures.

Any suitable method, including circular permutation (Yu and Lutz, Trends Biotechnol. 2011 January; 29(1):18-25), may be used to produce such variants. In circular permutation, the linear primary sequence of a polypeptide can be circularized (e.g., by joining the N-terminal and C-terminal ends of the sequence) and the polypeptide can be severed (“broken”) at a different location. Thus, the linear primary sequence of the new polypeptide may have low sequence identity (e.g., less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, less or less than 5%, including all values in between) as determined by linear sequence alignment methods (e.g., Clustal Omega or BLAST). Topological analysis of the two polypeptides, however, may reveal that their tertiary structure is similar. Without being bound by a particular theory, a variant polypeptide created through circular permutation of a reference polypeptide and with a tertiary structure similar to the reference polypeptide can share similar functional characteristics (e.g., enzymatic activity, enzyme kinetics, substrate specificity or product specificity). In some instances, circular permutation may alter the secondary structure, tertiary structure or quaternary structure and produce an enzyme with different functional characteristics (e.g., increased or decreased enzymatic activity, different substrate specificity, or different product specificity). See, e.g., Yu and Lutz, Trends Biotechnol. 2011 January; 29(1):18-25.

It should be appreciated that in a protein that has undergone circular permutation, the linear amino acid sequence of the protein would differ from a reference protein that has not undergone circular permutation. However, one of ordinary skill in the art would be able to readily determine which residues in the protein that has undergone circular permutation correspond to residues in the reference protein that has not undergone circular permutation by, for example, aligning the sequences and detecting conserved motifs, and/or by comparing the structures or predicted structures of the proteins, e.g., by homology modeling. Variants described in this application include circularly permutated variants of sequences described in this application.

In some embodiments, an algorithm that determines the percent identity between a sequence of interest and a reference sequence described in this application accounts for the presence of circular permutation between the sequences. The presence of circular permutation may be detected using any method known in the art, including, for example, RASPODOM (Weiner et al., Bioinformatics. 2005 Apr. 1; 21(7):932-7). In some embodiments, the presence of circulation permutation is corrected for (e.g., the domains in at least one sequence are rearranged) prior to calculation of the percent identity between a sequence of interest and a sequence described in this application. The claims of this application should be understood to encompass sequences for which percent identity to a reference sequence is calculated after taking into account potential circular permutation of the sequence.

Functional variants of the recombinant LeuDH, KivD, or Adh enzyme disclosed in this application are also encompassed by the present disclosure. For example, functional variants may bind one or more of the same substrates or produce one or more of the same products. Functional variants may be identified using any method known in the art. For example, the algorithm of Karlin and Altschul Proc. Natl. Acad. Sci. USA 87:2264-68, 1990 described above may be used to identify homologous proteins with known functions.

Putative functional variants may also be identified by searching for polypeptides with functionally annotated domains. Databases including Pfam (Sonnhammer et al., Proteins. 1997 July; 28(3):405-20) may be used to identify polypeptides with a particular domain.

Homology modeling may also be used to identify amino acid residues that are amenable to mutation without affecting function. A non-limiting example of such a method may include use of position-specific scoring matrix (PSSM) and an energy minimization protocol.

Position-specific scoring matrix (PSSM) uses a position weight matrix to identify consensus sequences (e.g., motifs). PSSM can be conducted on nucleic acid or amino acid sequences. Sequences are aligned and the method takes into account the observed frequency of a particular residue (e.g., an amino acid or a nucleotide) at a particular position and the number of sequences analyzed. See, e.g., Stormo et al., Nucleic Acids Res. 1982 May 11; 10(9):2997-3011. The likelihood of observing a particular residue at a given position can be calculated. Without being bound by a particular theory, positions in sequences with high variability may be amenable to mutation (e.g., PSSM score ≥0) to produce functional homologs.

PSSM may be paired with calculation of a Rosetta energy function, which determines the difference between the wild-type and the single-point mutant. The Rosetta energy function calculates this difference as (ΔΔG_{calc}). With the Rosetta function, the bonding interactions between a mutated residue and the surrounding atoms are used to determine whether a mutation increases or decreases protein stability. For example, a mutation that is designated as favorable by the PSSM score (e.g. PSSM score ≥0), can then be analyzed using the Rosetta energy function to determine the potential impact of the mutation on protein stability. Without being bound by a particular theory, potentially stabilizing mutations are desirable for protein engineering (e.g., production of functional homologs). In some embodiments, a potentially stabilizing mutation has a ΔΔG_{calc} value of less than −0.1 (e.g., less than −0.2, less than −0.3, less than −0.35, less than −0.4, less than −0.45, less than −0.5, less than −0.55, less than −0.6, less than −0.65, less than −0.7, less than −0.75, less than −0.8, less than −0.85, less than −0.9, less than −0.95, or less than −1.0) Rosetta energy units (R.e.u.). See, e.g., Goldenzweig et al., Mol Cell. 2016 Jul. 21; 63(2):337-346. Doi: 10.1016/j.molcel.2016.06.012.

In some embodiments, a LeuDH, KivD, or Adh enzyme coding sequence comprises a mutation at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more than 100 positions corresponding to a reference (e.g., LeuDH, KivD, or Adh enzyme) coding sequence. In some embodiments, the LeuDH, KivD, or Adh enzyme coding sequence comprises a mutation in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more codons of the coding sequence relative to a reference (e.g., LeuDH, KivD, or Adh enzyme) coding sequence. As will be understood by one of ordinary skill in the art, a mutation within a codon may or may not change the amino acid that is encoded by the codon due to degeneracy of the genetic code. In some embodiments, the one or more mutations in the coding sequence do not alter the amino acid sequence of the coding sequence (e.g., LeuDH, KivD, or Adh enzyme) relative to the amino acid sequence of a reference polypeptide (e.g., LeuDH, KivD, or Adh enzyme).

In some embodiments, the one or more mutations in a recombinant LeuDH, KivD, or Adh enzyme sequence alters the amino acid sequence of the polypeptide (e.g., LeuDH, KivD, or Adh enzyme) relative to the amino acid sequence of a reference polypeptide (e.g., LeuDH, KivD, or Adh enzyme). In some embodiments, the one or more mutations alters the amino acid sequence of the recombinant polypeptide (e.g., LeuDH, KivD, or Adh enzyme) relative to the amino acid sequence of a reference polypeptide (e.g., LeuDH, KivD, or Adh enzyme) and alters (enhances or reduces) an activity of the polypeptide relative to the reference polypeptide.

The activity (e.g., specific activity) of any of the recombinant polypeptides described in this disclosure (e.g., LeuDH, KivD, or Adh enzyme) may be measured using routine methods. As a non-limiting example, a recombinant polypeptide's activity may be determined by measuring its substrate specificity, product(s) produced, the concentration of product(s) produced, or any combination thereof. As used in this disclosure, “specific activity” of a recombinant polypeptide refers to the amount (e.g., concentration) of a particular product produced for a given amount (e.g., concentration) of the recombinant polypeptide per unit time.

The skilled artisan will also realize that mutations in a recombinant polypeptide (e.g., LeuDH, KivD, or Adh enzyme) coding sequence may result in conservative amino acid substitutions to provide functionally equivalent variants of the foregoing polypeptides, e.g., variants that retain the activities of the polypeptides. As used in this disclosure, a “conservative amino acid substitution” refers to an amino acid substitution that does not alter the relative charge or size characteristics or functional activity of the protein in which the amino acid substitution is made.

In some instances, an amino acid is characterized by its R group (see, e.g., Table 1). For example, an amino acid may comprise a nonpolar aliphatic R group, a positively charged R group, a negatively charged R group, a nonpolar aromatic R group, or a polar uncharged R group. Non-limiting examples of an amino acid comprising a nonpolar aliphatic R group include alanine, glycine, valine, leucine, methionine, and isoleucine. Non-limiting examples of an amino acid comprising a positively charged R group include lysine, arginine, and histidine. Non-limiting examples of an amino acid comprising a negatively charged R group include aspartate and glutamate. Non-limiting examples of an amino acid comprising a nonpolar, aromatic R group include phenylalanine, tyrosine, and tryptophan. Non-limiting examples of an amino acid comprising a polar uncharged R group include serine, threonine, cysteine, proline, asparagine, and glutamine.

Variants can be prepared according to methods for altering polypeptide sequence known to one of ordinary skill in the art such as are found in references which compile such methods, e.g., Molecular Cloning: A Laboratory Manual, J. Sambrook, et al., eds., Fourth Edition, Cold Spring Harbor Laboratory Press, Cold Spring Harbor, N.Y., 2012, or Current Protocols in Molecular Biology, F. M. Ausubel, et al., eds., John Wiley & Sons, Inc., New York, 2010.

Non-limiting examples of functionally equivalent variants of polypeptides may include conservative amino acid substitutions in the amino acid sequences of proteins disclosed in this application. As used in this disclosure “conservative substitution” is used interchangeably with “conservative amino acid substitution” and refers to any one of the amino acid substitutions provided in Table 1.

In some embodiments, 1, 2, 3, 4, 5, 6, 7, 8, 9, 10, 11, 12, 13, 14, 15, 16, 17, 18, 19, 20 or more than 20 residues can be changed when preparing variant polypeptides. In some embodiments, amino acids are replaced by conservative amino acid substitutions.

TABLE 1
Conservative Amino Acid Substitutions.

| Original Residue | R Group Type | Conservative Amino Acid Substitutions |
|---|---|---|
| Ala | nonpolar aliphatic R group | Cys, Gly, Ser |
| Arg | positively charged R group | His, Lys |
| Asn | polar uncharged R group | Asp, Gln, Glu |
| Asp | negatively charged R group | Asn, Gln, Glu |
| Cys | polar uncharged R group | Ala, Ser |
| Gln | polar uncharged R group | Asn, Asp, Glu |
| Glu | negatively charged R group | Asn, Asp, Gln |
| Gly | nonpolar aliphatic R group | Ala, Ser |
| His | positively charged R group | Arg, Tyr, Trp |
| Ile | nonpolar aliphatic R group | Leu, Met, Val |
| Leu | nonpolar aliphatic R group | Ile, Met, Val |
| Lys | positively charged R group | Arg, His |
| Met | nonpolar aliphatic R group | Ile, Leu, Phe, Val |
| Pro | polar uncharged R group | |
| Phe | nonpolar aromatic R group | Met, Trp, Tyr |
| Ser | polar uncharged R group | Ala, Gly, Thr |
| Thr | polar uncharged R group | Ala, Asn, Ser |
| Trp | nonpolar aromatic R group | His, Phe, Tyr, Met |
| Tyr | nonpolar aromatic R group | His, Phe, Trp |
| Val | nonpolar aliphatic R group | Ile, Leu, Met, Thr |

Amino acid substitutions in the amino acid sequence of a polypeptide to produce a recombinant polypeptide (e.g., LeuDH, KivD, or Adh enzyme) variant having a desired property and/or activity can be made by alteration of the coding sequence of the polypeptide (e.g., LeuDH, KivD, or Adh enzyme). Similarly, conservative amino acid substitutions in the amino acid sequence of a polypeptide to produce functionally equivalent variants of the polypeptide typically are made by alteration of the coding sequence of the recombinant polypeptide (e.g., LeuDH, KivD, or Adh enzyme).

Mutations (e.g., substitutions) can be made in a nucleotide sequence by a variety of methods known to one of ordinary skill in the art. For example, mutations can be made by PCR-directed mutation, site-directed mutagenesis according to the method of Kunkel (Kunkel, Proc. Nat. Acad. Sci. U.S.A. 82: 488-492, 1985), by chemical synthesis of a gene encoding a polypeptide, by gene editing techniques, or by insertions, such as insertion of a tag (e.g., a HIS tag or a GFP tag).

### Nucleic Acids Encoding Branched-Chain Amino Acid (BCAA) Pathway Enzymes

Aspects of the present disclosure relate to recombinant enzymes, functional modifications and variants thereof, as well as uses relating thereto. For example, the enzymes and cells described in this application may be used to promote leucine consumption, e.g., by converting leucine to isopentanol. The methods may comprise using a host cell comprising one or more enzymes disclosed in this application, a cell lysate, isolated enzymes, or any combination thereof. Methods comprising recombinant expression of polynucleotides encoding an enzyme disclosed in this application in a host cell are encompassed by the present disclosure. Methods comprising administering a host cell comprising at least one BCAA pathway enzyme (e.g., LeuDH, KivD, or Adh enzyme) to a subject in need thereof are encompassed by the present disclosure. In vitro methods comprising reacting one or more branched-chain amino acids (BCAAs) in a reaction mixture with a BCAA pathway enzyme disclosed in this application are also encompassed by the present disclosure. In some embodiments, the BCAA pathway enzyme is an LeuDH, KivD, or Adh enzyme, or a combination thereof.

A nucleic acid encoding any one or more of the recombinant polypeptides (e.g., LeuDH, KivD, Adh, and/or BrnQ) is encompassed by the disclosure and may be comprised within a host cell. In some embodiments, the nucleic acid is in the form of an operon. In some embodiments, at least one ribosome binding site is present between one or more of the coding sequences present in the nucleic acid.

In some embodiments, LeuDH, KivD, Adh, and/or BrnQ nucleic acid sequences encompassed by the disclosure are nucleic acid sequences that hybridize to a LeuDH, KivD, Adh, and/or BrnQ nucleic acid sequence provided in this disclosure under high or medium stringency conditions and that are biologically active. For example, nucleic acids that hybridize under high stringency conditions of 0.2 to 1×SSC at 65° C. followed by a wash at 0.2×SSC at 65° C. to a nucleic acid encoding LeuDH, KivD, Adh, and/or BrnQ can be used. Nucleic acids that hybridize under low stringency conditions of 6×SSC at room temperature followed by a wash at 2×SSC at room temperature to a nucleic acid encoding LeuDH, KivD, Adh, and/or BrnQ can be used. Other hybridization conditions include 3×SSC at 40° C. or 50° C., followed by a wash in 1 or 2×SSC at 20° C., 30° C., 40° C., 50° C., 60° C., or 65° C.

Hybridizations can be conducted in the presence of formaldehyde, e.g., 10%, 20%, 30% 40% or 50%, which further increases the stringency of hybridization. Theory and practice of nucleic acid hybridization is described, e.g., in S. Agrawal (ed.) Methods in Molecular Biology, volume 20; and Tijssen (1993) Laboratory Techniques in biochemistry and molecular biology-hybridization with nucleic acid probes, e.g., part I chapter 2 “Overview of principles of hybridization and the strategy of nucleic acid probe assays,” Elsevier, New York provide a basic guide to nucleic acid hybridization. Exemplary proteins may have at least about 50%, 70%, 80%, 90%, preferably at least about 95%, even more preferably at least about 98% and most preferably at least 99% homology or identity with a LeuDH, KivD, or Adh protein or a domain thereof, e.g., the catalytic domain. Other exemplary proteins may be encoded by a nucleic acid that is at least about 90%, preferably at least about 95%, even more preferably at least about 98% and most preferably at least 99% homology or identity with a LeuDH, KivD, or Adh nucleic acid, e.g., those described in this application.

A nucleic acid encoding any one or more of the recombinant polypeptides (e.g., LeuDH, KivD, Adh and/or BrnQ) described in this application may be incorporated into any appropriate vector through any method known in the art. For example, the vector may be an expression vector, including but not limited to a viral vector (e.g., a lentiviral, retroviral, adenoviral, or adeno-associated viral vector), any vector suitable for transient expression, any vector suitable for constitutive expression, or any vector suitable for inducible expression (e.g., a galactose-inducible or doxycycline-inducible vector).

In some embodiments, a vector replicates autonomously in the cell. In some embodiments, a vector integrates into a chromosome within a cell. A vector can contain one or more endonuclease restriction sites that are cut by a restriction endonuclease to insert and ligate a nucleic acid containing a gene described in this application to produce a recombinant vector that is able to replicate in a cell. Vectors are typically composed of DNA, although RNA vectors are also available. Cloning vectors include, but are not limited to: plasmids, fosmids, phagemids, virus genomes and artificial chromosomes. As used in this application, the terms “expression vector” or “expression construct” refer to a nucleic acid construct, generated recombinantly or synthetically, with a series of specified nucleic acid elements that permit transcription of a particular nucleic acid in a host cell (e.g., microbe), such as a yeast cell. In some embodiments, the nucleic acid sequence of a gene described in this application is inserted into a cloning vector such that it is operably joined to regulatory sequences and, in some embodiments, expressed as an RNA transcript. In some embodiments, the vector contains one or more markers, such as a selectable marker as described in this application, to identify cells transformed or transfected with the recombinant vector. In some embodiments, the nucleic acid sequence of a gene described in this application is codon-optimized. Codon optimization may increase production of the gene product by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%, including all values in between) relative to a reference sequence that is not codon-optimized.

In some embodiments, nucleic acid sequences described in this application are expressed in plasmids. For example, nucleic acid sequences described in this application may be expressed in cloning plasmids. Nucleic acid sequences described in this application may be expressed in plasmids for transient expression. Nucleic acid sequences described in this application may also be expressed in plasmids for incorporation of the nucleic acid sequences into genomic DNA.

A coding sequence and a regulatory sequence are said to be “operably joined” or “operably linked” when the coding sequence and the regulatory sequence are covalently linked and the expression or transcription of the coding sequence is under the influence or control of the regulatory sequence. If the coding sequence is to be translated into a functional protein, the coding sequence and the regulatory sequence are said to be operably joined if induction of a promoter in the 5′ regulatory sequence permits the coding sequence to be transcribed and if the nature of the linkage between the coding sequence and the regulatory sequence does not (1) result in the introduction of a frame-shift mutation, (2) interfere with the ability of the promoter region to direct the transcription of the coding sequence, or (3) interfere with the ability of the corresponding RNA transcript to be translated into a protein.

In some embodiments, the nucleic acid encoding any one or more of the proteins described in this application is under the control of regulatory sequences (e.g., enhancer sequences). In some embodiments, a nucleic acid is expressed under the control of a promoter. The promoter can be a native promoter, e.g., the promoter of the gene in its endogenous context, which provides normal regulation of expression of the gene.

Alternatively, a promoter can be a promoter that is different from the native promoter of the gene, e.g., the promoter is different from the promoter of the gene in its endogenous context. In some embodiments, the promoter is a eukaryotic promoter. Non-limiting examples of eukaryotic promoters include TDH3, PGK1, PKC1, PDC1, TEF1, TEF2, RPL18B, SSA1, TDH2, PYK1,TPI1 GAL1, GAL10, GALT, GAL3, GAL2, MET3, MET25, HXT3, HXT7, ACT1, ADH1, ADH2, CUP1-1, ENO2, and SOD1, as would be known to one of ordinary skill in the art (see, e.g., Addgene website: blog.addgene.org/plasmids-101-the-promoter-region). In some embodiments, the promoter is a prokaryotic promoter (e.g., bacteriophage or bacterial promoter). Non-limiting examples of bacteriophage promoters include Pls1con, T3, T7, SP6, and PL. Non-limiting examples of bacterial promoters include Pbad, PmgrB, Ptrc2, PCI857, Plac/ara, Plac/fnr, Ptac, Ptet, Pcmt, and Pm.

In some embodiments, the promoter is an inducible promoter. As used in this application, an “inducible promoter” is a promoter controlled by the presence or absence of a molecule. This may be used, for example, to controllably induce the expression of an enzyme. In some embodiments, where an inducible promoter is linked to a LeuDH, a KivD and/or a Adh, the expression of LeuDH, KivD and/or Adh may be induced or not induced at certain times. For example, in some embodiments, expression may not be induced at certain times so that leucine consumption would be limited (e.g., during cell growth). Non-limiting examples of inducible promoters include chemically regulated promoters and physically regulated promoters. For chemically regulated promoters, the transcriptional activity can be regulated by one or more compounds, such as alcohol, tetracycline, galactose, a steroid, a metal, or other compounds. For physically regulated promoters, transcriptional activity can be regulated by a phenomenon such as light or temperature. Non-limiting examples of tetracycline-regulated promoters include anhydrotetracycline (aTc)-responsive promoters and other tetracycline-responsive promoter systems (e.g., a tetracycline repressor protein (tetR), a tetracycline operator sequence (tetO) and a tetracycline transactivator fusion protein (tTA)). Non-limiting examples of steroid-regulated promoters include promoters based on the rat glucocorticoid receptor, human estrogen receptor, moth ecdysone receptors, and promoters from the steroid/retinoid/thyroid receptor superfamily. Non-limiting examples of metal-regulated promoters include promoters derived from metallothionein (proteins that bind and sequester metal ions) genes. Non-limiting examples of pathogenesis-regulated promoters include promoters induced by salicylic acid, ethylene or benzothiadiazole (BTH). Non-limiting examples of temperature/heat-inducible promoters include heat shock promoters. Non-limiting examples of light-regulated promoters include light responsive promoters from plant cells. In certain embodiments, the inducible promoter is a galactose-inducible promoter. In some embodiments, the inducible promoter is induced by one or more physiological conditions (e.g., pH, temperature, radiation, osmotic pressure, saline gradients, cell surface binding, or concentration of one or more extrinsic or intrinsic inducing agents). Non-limiting examples of an extrinsic inducer or inducing agent include amino acids and amino acid analogs, saccharides and polysaccharides, nucleic acids, protein transcriptional activators and repressors, cytokines, toxins, petroleum-based compounds, metal containing compounds, salts, ions, enzyme substrate analogs, hormones or any combination thereof.

In some embodiments, the promoter is a constitutive promoter. As used in this application, a “constitutive promoter” refers to an unregulated promoter that allows continuous transcription of a gene. Non-limiting examples of a constitutive promoter include TDH3, PGK1, PKC1, PDC1, TEF1, TEF2, RPL18B, SSA1, TDH2, PYK1,TPI1, HXT3, HXT7, ACT1, ADH1, ADH2, ENO2, and SOD1.

Other inducible promoters or constitutive promoters known to one of ordinary skill in the art are also contemplated in this application.

The precise nature of the regulatory sequences needed for gene expression may vary between species or cell types, but generally include, as necessary, 5′ non-transcribed and 5′ non-translated sequences involved with the initiation of transcription and translation respectively, such as a TATA box, capping sequence, CAAT sequence, and the like. In particular, such 5′ non-transcribed regulatory sequences will include a promoter region which includes a promoter sequence for transcriptional control of the operably joined gene. Regulatory sequences may also include enhancer sequences or upstream activator sequences. The vectors disclosed in this application may include 5′ leader or signal sequences. Regulatory sequences may also include a terminator sequence. In some embodiments, a terminator sequence marks the end of a gene in DNA during transcription. The choice and design of one or more appropriate vectors suitable for inducing expression of one or more genes described in this application in a heterologous organism is within the ability and discretion of one of ordinary skill in the art.

Expression vectors containing the necessary elements for expression are commercially available and known to one of ordinary skill in the art (see, e.g., Sambrook et al., Molecular Cloning: A Laboratory Manual, Fourth Edition, Cold Spring Harbor Laboratory Press, 2012).

### Host Cells

The disclosed methods and compositions and host cells are exemplified with E. coli cells (e.g., E. coli Nissle 1917), but are, in some embodiments, applicable to other host cells.

Suitable host cells include, but are not limited to: yeast cells, bacterial cells, algal cells, plant cells, fungal cells, insect cells, and animal cells, including mammalian cells. In one illustrative embodiment, suitable host cells include E. coli (e.g., Shuffle™ competent E. coli available from New England BioLabs in Ipswich, Mass. or E. coli Nissle 1917 available from German Collection of Microorganisms and Cell Cultures (DSMZ Braunschweig, E. coli DSM 6601)).

Suitable yeast host cells include, but are not limited to: Candida, Hansenula, Saccharomyces, Schizosaccharomyces, Pichia, Kluyveromyces, and Yarrowia. In some embodiments, the yeast cell is Hansenula polymorpha, Saccharomyces cerevisiae, Saccaromyces carlsbergensis, Saccharomyces diastaticus, Saccharomyces norbensis, Saccharomyces kluyveri, Schizosaccharomyces pombe, Pichia pastoris, Pichia finlandica, Pichia trehalophila, Pichia kodamae, Pichia membranaefaciens, Pichia opuntiae, Pichia thermotolerans, Pichia salictaria, Pichia quercuum, Pichia pijperi, Pichia stipitis, Pichia methanolica, Pichia angusta, Kluyveromyces lactis, Candida albicans, or Yarrowia lipolytica.

In some embodiments, the yeast strain is an industrial polyploid yeast strain. Other non-limiting examples of fungal cells include cells obtained from Aspergillus spp., Penicillium spp., Fusarium spp., Rhizopus spp., Acremonium spp., Neurospora spp., Sordaria spp., Magnaporthe spp., Allomyces spp., Ustilago spp., Botrytis spp., and Trichoderma spp.

In certain embodiments, the host cell is an algal cell such as Chlamydomonas (e.g., C. Reinhardtii) and Phormidium (P. sp. ATCC29409).

In other embodiments, the host cell is a prokaryotic cell. Suitable prokaryotic cells include gram positive, gram negative, and gram-variable bacterial cells. The host cell may be a species of, but not limited to: Agrobacterium, Alicyclobacillus, Anabaena, Anacystis, Acinetobacter, Acidothermus, Arthrobacter, Azobacter, Bacillus, Bifidobacterium, Brevibacterium, Butyrivibrio, Buchnera, Campestris, Campylobacter, Clostridium, Corynebacterium, Chromatium, Coprococcus, Escherichia, Enterococcus, Enterobacter, Erwinia, Fusobacterium, Faecalibacterium, Francisella, Flavobacterium, Geobacillus, Haemophilus, Helicobacter, Klebsiella, Lactobacillus, Lactococcus, Ilyobacter, Micrococcus, Microbacterium, Mesorhizobium, Methylobacterium, Methylobacterium, Mycobacterium, Neisseria, Pantoea, Pseudomonas, Prochlorococcus, Rhodobacter, Rhodopseudomonas, Rhodopseudomonas, Roseburia, Rhodospirillum, Rhodococcus, Scenedesmus, Streptomyces, Streptococcus, Synecoccus, Saccharomonospora, Saccharopolyspora, Staphylococcus, Serratia, Salmonella, Shigella, Thermoanaerobacterium, Tropheryma, Tularensis, Temecula, Thermosynechococcus, Thermococcus, Ureaplasma, Xanthomonas, Xylella, Yersinia, and Zymomonas.

In some embodiments, the bacterial host strain is an industrial strain. Numerous bacterial industrial strains are known and suitable for the methods and compositions described in this application.

In some embodiments, the bacterial host cell is of the Agrobacterium species (e.g., A. radiobacter, A. rhizogenes, A. rubi), the Arthrobacterspecies (e.g., A. aurescens, A. citreus, A. globformis, A. hydrocarboglutamicus, A. mysorens, A. nicotianae, A. paraffineus, A. protophonniae, A. roseoparaffinus, A. sulfureus, A. ureafaciens), the Bacillus species (e.g., B. thuringiensis, B. anthracis, B. megaterium, B. subtilis, B. lentus, B. circulars, B. pumilus, B. lautus, B. coagulans, B. brevis, B. firmus, B. alkaophius, B. licheniformis, B. clausii, B. stearothermophilus, B. halodurans and B. amyloliquefaciens. In particular embodiments, the host cell will be an industrial Bacillus strain including but not limited to B. subtilis, B. pumilus, B. licheniformis, B. megaterium, B. clausii, B. stearothermophilus and B. amyloliquefaciens. In some embodiments, the host cell will be an industrial Clostridium species (e.g., C. acetobutylicum, C. tetani E88, C. lituseburense, C. saccharobutylicum, C. perfringens, C. beijerinckii). In some embodiments, the host cell will be an industrial Corynebacterium species (e.g., C. glutamicum, C. acetoacidophilum). In some embodiments, the host cell will be an industrial Escherichia species (e.g., E. coli). In some embodiments, the host cell will be an industrial Erwinia species (e.g., E. uredovora, E. carotovora, E. ananas, E. herbicola, E. punctata, E. terreus). In some embodiments, the host cell will be an industrial Pantoea species (e.g., P. citrea, P. agglomerans). In some embodiments, the host cell will be an industrial Pseudomonas species, (e.g., P. putida, P. aeruginosa, P. mevalonii). In some embodiments, the host cell will be an industrial Streptococcus species (e.g., S. equisimiles, S. pyogenes, S. uberis). In some embodiments, the host cell will be an industrial Streptomyces species (e.g., S. ambofaciens, S. achromogenes, S. avermitilis, S. coelicolor, S. aureofaciens, S. aureus, S. fungicidicus, S. griseus, S. lividans). In some embodiments, the host cell will be an industrial Zymomonas species (e.g., Z. mobilis, Z. lipolytica), and the like.

The present disclosure is also suitable for use with a variety of animal cell types, including mammalian cells, for example, human (including 293, HeLa, WI38, PER.C6 and Bowes melanoma cells), mouse (including 3T3, NS0, NS1, Sp2/0), hamster (CHO, BHK), monkey (COS, FRhL, Vero), and hybridoma cell lines.

In various embodiments, strains that may be used in the practice of the disclosure including both prokaryotic and eukaryotic strains, and are readily accessible to the public from a number of culture collections such as American Type Culture Collection (ATCC), Deutsche Sammlung von Mikroorganismen and Zellkulturen GmbH (DSM), Centraalbureau Voor Schimmelcultures (CBS), and Agricultural Research Service Patent Culture Collection, Northern Regional Research Center (NRRL). The present disclosure is also suitable for use with a variety of plant cell types.

The term “cell,” as used in this application, may refer to a single cell or a population of cells, such as a population of cells belonging to the same cell line or strain. Use of the singular term “cell” should not be construed to refer explicitly to a single cell rather than a population of cells. The host cell may comprise genetic modifications relative to a wild-type counterpart.

A vector encoding any one or more of the recombinant polypeptides (e.g., LeuDH, KivD, Adh enzyme and/or BrnQ) described in this application may be introduced into a suitable host cell using any method known in the art. Host cells may be cultured under any conditions suitable as would be understood by one of ordinary skill in the art. For example, any media, temperature, and incubation conditions known in the art may be used. For host cells carrying an inducible vector, cells may be cultured with an appropriate inducible agent to promote expression.

Any of the cells disclosed in this application can be cultured in media of any type (rich or minimal) and any composition prior to, during, and/or after contact and/or integration of a nucleic acid. The conditions of the culture or culturing process can be optimized through routine experimentation as would be understood by one of ordinary skill in the art. In some embodiments, the selected media is supplemented with various components. In some embodiments, the concentration and amount of a supplemental component is optimized. In some embodiments, other aspects of the media and growth conditions (e.g., pH, temperature, etc.) are optimized through routine experimentation. In some embodiments, the frequency that the media is supplemented with one or more supplemental components, and the amount of time that the cell is cultured, is optimized.

Culturing of the cells described in this application can be performed in culture vessels known and used in the art. In some embodiments, an aerated reaction vessel (e.g., a stirred tank reactor) is used to culture the cells. In some embodiments, a bioreactor or fermentor is used to culture the cell. Thus, in some embodiments, the cells are used in fermentation. As used in this application, the terms “bioreactor” and “fermentor” are interchangeably used and refer to an enclosure, or partial enclosure, in which a biological, biochemical and/or chemical reaction takes place, involving a living organism or part of a living organism. A “large-scale bioreactor” or “industrial-scale bioreactor” is a bioreactor that is used to generate a product on a commercial or quasi-commercial scale. Large scale bioreactors typically have volumes in the range of liters, hundreds of liters, thousands of liters, or more.

In some embodiments, a bioreactor comprises a cell (e.g., a bacterial cell) or a cell culture (e.g., a bacterial cell culture), such as a cell or cell culture described in this application. In some embodiments, a bioreactor comprises a spore and/or a dormant cell type of an isolated microbe (e.g., a dormant cell in a dry state).

Non-limiting examples of bioreactors include: stirred tank fermentors, bioreactors agitated by rotating mixing devices, chemostats, bioreactors agitated by shaking devices, airlift fermentors, packed-bed reactors, fixed-bed reactors, fluidized bed bioreactors, bioreactors employing wave induced agitation, centrifugal bioreactors, roller bottles, and hollow fiber bioreactors, roller apparatuses (for example benchtop, cart-mounted, and/or automated varieties), vertically-stacked plates, spinner flasks, stirring or rocking flasks, shaken multi-well plates, MD bottles, T-flasks, Roux bottles, multiple-surface tissue culture propagators, modified fermentors, and coated beads (e.g., beads coated with serum proteins, nitrocellulose, or carboxymethyl cellulose to prevent cell attachment).

In some embodiments, the bioreactor includes a cell culture system where the cell (e.g., bacterial cell) is in contact with moving liquids and/or gas bubbles. In some embodiments, the cell or cell culture is grown in suspension. In other embodiments, the cell or cell culture is attached to a solid phase carrier. Non-limiting examples of a carrier system includes microcarriers (e.g., polymer spheres, microbeads, and microdisks that can be porous or non-porous), cross-linked beads (e.g., dextran) charged with specific chemical groups (e.g., tertiary amine groups), 2D microcarriers including cells trapped in nonporous polymer fibers, 3D carriers (e.g., carrier fibers, hollow fibers, multicartridge reactors, and semi-permeable membranes that can comprising porous fibers), microcarriers having reduced ion exchange capacity, encapsulation cells, capillaries, and aggregates. In some embodiments, carriers are fabricated from materials such as dextran, gelatin, glass, or cellulose.

In some embodiments, industrial-scale processes are operated in continuous, semi-continuous or non-continuous modes. Non-limiting examples of operation modes are batch, fed batch, extended batch, repetitive batch, draw/fill, rotating-wall, spinning flask, and/or perfusion mode of operation. In some embodiments, a bioreactor allows continuous or semi-continuous replenishment of the substrate stock, for example a carbohydrate source and/or continuous or semi-continuous separation of the product, from the bioreactor.

In some embodiments, the bioreactor or fermentor includes a sensor and/or a control system to measure and/or adjust reaction parameters. Non-limiting examples of reaction parameters include biological parameters (e.g., growth rate, cell size, cell number, cell density, cell type, or cell state, etc.), chemical parameters (e.g., pH, redox-potential, concentration of reaction substrate and/or product, concentration of dissolved gases, such as oxygen concentration and CO₂ concentration, nutrient concentrations, metabolite concentrations, concentration of an oligopeptide, concentration of an amino acid, concentration of a vitamin, concentration of a hormone, concentration of an additive, serum concentration, ionic strength, concentration of an ion, relative humidity, molarity, osmolarity, concentration of other chemicals, for example buffering agents, adjuvants, or reaction by-products), physical/mechanical parameters (e.g., density, conductivity, degree of agitation, pressure, and flow rate, shear stress, shear rate, viscosity, color, turbidity, light absorption, mixing rate, conversion rate, as well as thermodynamic parameters, such as temperature, light intensity/quality, etc.). Sensors to measure the parameters described in this application are well known to one of ordinary skill in the relevant mechanical and electronic arts. Control systems to adjust the parameters in a bioreactor based on the inputs from a sensor described in this application are well known to one of ordinary skill in the art in bioreactor engineering.

In some embodiments, the method involves batch fermentation (e.g., shake flask fermentation). General considerations for batch fermentation (e.g., shake flask fermentation) include the level of oxygen and glucose. For example, batch fermentation (e.g., shake flask fermentation) may be oxygen and glucose limited, so in some embodiments, the capability of a strain to perform in a well-designed fed-batch fermentation is underestimated. Also, the final product may display some differences from the substrate in terms of solubility, toxicity, cellular accumulation and secretion and in some embodiments can have different fermentation kinetics.

In some embodiments, the cells of the present disclosure are adapted to consume leucine in vivo. In some embodiments, the cells are adapted to produce one or more enzymes for leucine consumption via conversion to isopentanol (e.g., LeuDH, KivD, and/or Adh). In such embodiments, the enzyme can catalyze reactions for the consumption of leucine by bioconversion in an in vitro or ex vivo process.

Any of the proteins or enzymes of the present disclosure may be expressed in a host cell. As used in this application, a host cell is a cell that can be used to express at least one heterologous polynucleotide (e.g., encoding a protein or enzyme as described in this application). The term “heterologous” with respect to a polynucleotide, such as a polynucleotide comprising a gene, is used interchangeably with the term “exogenous” and the term “recombinant” and refers to: a polynucleotide that has been artificially supplied to a biological system; a polynucleotide that has been modified within a biological system, or a polynucleotide whose expression or regulation has been manipulated within a biological system. A heterologous polynucleotide that is introduced into or expressed in a host cell may be a polynucleotide that comes from a different organism or species than the host cell, or may be a synthetic polynucleotide, or may be a polynucleotide that is also endogenously expressed in the same organism or species as the host cell. For example, a polynucleotide that is endogenously expressed in a host cell may be considered heterologous when it is situated non-naturally in the host cell; expressed recombinantly in the host cell, either stably or transiently; modified within the host cell; selectively edited within the host cell; expressed in a copy number that differs from the naturally occurring copy number within the host cell; or expressed in a non-natural way within the host cell, such as by manipulating regulatory regions that control expression of the polynucleotide. In some embodiments, a heterologous polynucleotide is a polynucleotide that is endogenously expressed in a host cell but whose expression is driven by a promoter that does not naturally regulate expression of the polynucleotide. In other embodiments, a heterologous polynucleotide is a polynucleotide that is endogenously expressed in a host cell and whose expression is driven by a promoter that does naturally regulate expression of the polynucleotide, but the promoter or another regulatory region is modified. In some embodiments, the promoter is recombinantly activated or repressed. For example, gene-editing based techniques may be used to regulate expression of a polynucleotide, including an endogenous polynucleotide, from a promoter, including an endogenous promoter. See, e.g., Chavez et al., Nat Methods. 2016 July; 13(7): 563-567. A heterologous polynucleotide may comprise a wild-type sequence or a mutant sequence as compared with a reference polynucleotide sequence.

Any suitable host cell may be used to produce any of the recombinant polypeptides (e.g., LeuDH, KivD, and/or Adh) disclosed in this application, including eukaryotic cells or prokaryotic cells.

### Compositions

The present disclosure provides compositions, including pharmaceutical compositions, comprising a host cell described in this application (e.g., a host cell comprising a heterologous polynucleotide encoding at least one enzyme selected from the group consisting of LeuDH, KivD, and Adh) or one or more enzymes described in this application (e.g., LeuDH, KivD, and/or Adh), and optionally a pharmaceutically acceptable excipient.

In certain embodiments, a host cell described in this application is provided in an effective amount in a composition, such as a pharmaceutical composition. In certain embodiments, one or more enzymes described in this application are provided in an effective amount in a composition, such as a pharmaceutical composition. In certain embodiments, the effective amount is a therapeutically effective amount. In certain embodiments, the effective amount is a prophylactically effective amount. In some embodiments, the effective amount is an amount that is sufficient to treat or ameliorate one or more symptoms of MSUD.

In certain embodiments, the subject is an animal. In certain embodiments, the subject is a human. In other embodiments, the subject is a non-human animal. In certain embodiments, the subject is a mammal. In certain embodiments, the subject is a non-human mammal. In some embodiments, the subject is a non-mammal. In certain embodiments, the subject is a domesticated animal, such as a dog, cat, cow, pig, horse, sheep, chicken or goat. In certain embodiments, the subject is a companion animal, such as a dog or cat. In certain embodiments, the subject is a livestock animal, such as a cow, pig, horse, sheep, chicken, or goat. In certain embodiments, the subject is a zoo animal. In another embodiment, the subject is a research animal, such as a rodent (e.g., mouse, rat), dog, pig, or non-human primate.

Compositions, such as pharmaceutical compositions, described in this application can be prepared by any method known in the art. In general, such preparatory methods include bringing a compound described in this application (e.g., the “active ingredient”) into association with a carrier or excipient, and/or one or more other accessory ingredients, and then, if necessary and/or desirable, shaping, and/or packaging the product into a desired single- or multi-dose unit.

### Methods

In some aspects, the disclosure provides methods of using host cells. In some embodiments, the disclosure provides a method comprising culturing a host cell described in this application (e.g., a host cell comprising a heterologous polynucleotide encoding at least one enzyme selected from the group consisting of LeuDH, KivD, and Adh). Methods for culturing cells are described elsewhere in this application. In some embodiments, the disclosure provides a method of producing isopentanol from leucine comprising culturing a host cell described in this application (e.g., a host cell comprising a heterologous polynucleotide encoding LeuDH, KivD, and Adh). In some embodiments, the production and culturing occurs in vivo, e.g., in a human subject that has been administered the host cell. In some embodiments, the production occurs ex vivo, e.g., in an in vitro cell culture environment. Compositions, cells, enzymes, and methods described in this application are also applicable to industrial settings, including any application wherein there may be a buildup of branched-chain amino acids (e.g., leucine, isoleucine, and valine).

The present invention is further illustrated by the following Examples, which in no way should be construed as limiting. The entire contents of all of the references (including literature references, issued patents, published patent applications, and co pending patent applications) cited throughout this application are hereby expressly incorporated by reference. If a reference incorporated in this application contains a term whose definition is incongruous or incompatible with the definition of same term as defined in the present disclosure, the meaning ascribed to the term in this disclosure shall govern. However, mention of any reference, article, publication, patent, patent publication, and patent application cited in this application is not, and should not be taken as an acknowledgment or any form of suggestion that they constitute valid prior art or form part of the common general knowledge in any country in the world.

### EXAMPLES

In order that the invention described in this application may be more fully understood, the following examples are set forth. The examples described in this application are offered to illustrate the systems and methods provided in this application and are not to be construed in any way as limiting their scope.

### Example 1: Enzyme Library Design and Synthesis

### Materials and Methods

### Metagenomic Enzyme Discovery

Machine-learning-based bioinformatics tools were used to identify enzyme candidates for each of the three desired activities (leucine dehydrogenase, 1.4.1.9; ketoisovalerate decarboxylase, 4.1.1.1; and alcohol dehydrogenase 1.1.1.1) in public sequence databases (SwissProt and TrEMBL, together known as UniProt). For LeuDH and Adh, sequence diversity was maximized using previously developed algorithms. For KivD, a stratified sampling approach was used. The total number of enzyme candidates were 1175 LeuDH sequences, 1296 KivD sequences and 1177 Adh sequences.

### Rational Enzyme Design

For LeuDH and Adh, molecular models of the enzyme—transition state complex were built using Rosetta software, and systematic mutations of the active site residues to each of the 20 amino acids were designed.

### Library Synthesis

DNA sequences for all LeuDH, KivD, and Adh enzymes were codon optimized for expression in E. coli. Coding sequences were synthesized in an inducible E. coli expression vector under the control of the T7 promoter.

### Results

To improve the leucine-consuming branched-chain amino acid (BCAA) pathway, experiments were performed to identify LeuDH, KivD, and Adh enzymes with superior activity relative to parent enzymes in a prototype strain (1980, also known as SYN1980), which parent strain included Bacillus cereus LeuDH, Lactococcus lactis KivD, and Saccharomyces cerevisiae ADH2. The prototype strain also included BrnQ from E. coli, which is a transporter for branched-chain amino acids that can transport branched-chain amino acids, such as leucine, into the cell. The parent LeuDH enzyme exhibited substrate promiscuity, deaminating valine and isoleucine in addition to leucine. To improve specific consumption of leucine by the BCAA pathway, an additional goal for the pathway design was to identify LeuDH enzymes with increased specificity for leucine (Leu) relative to valine (Val) and isoleucine (Ile).

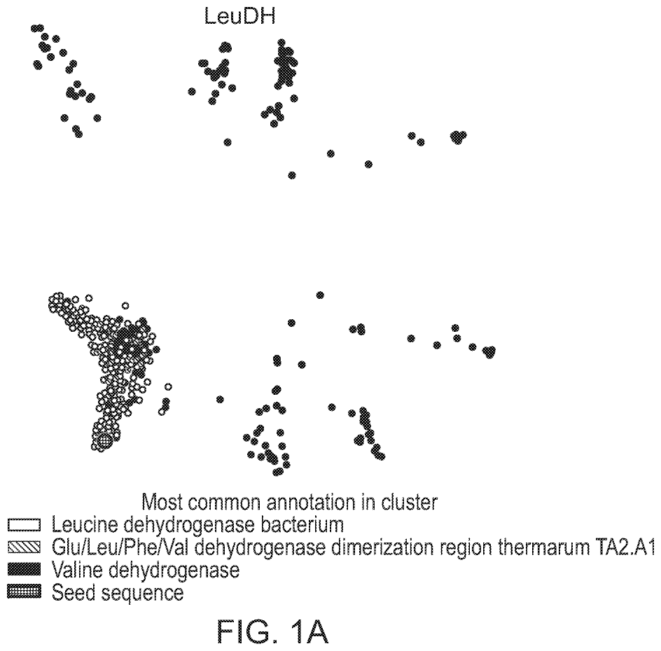

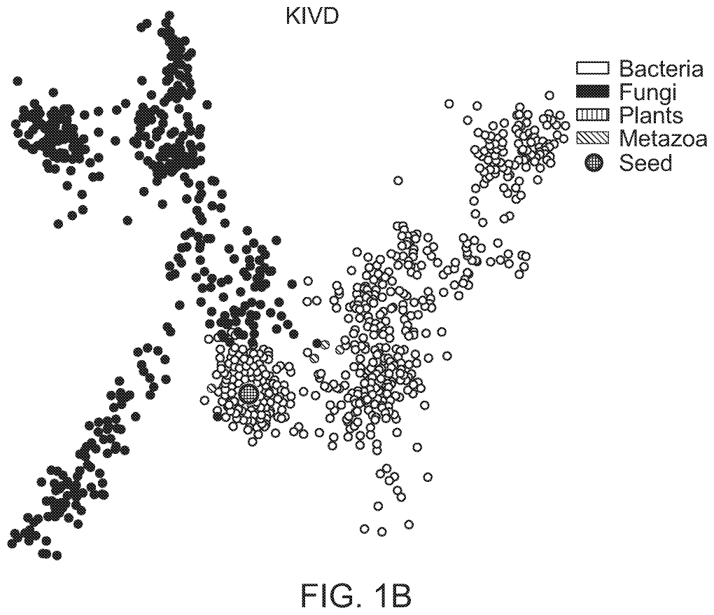

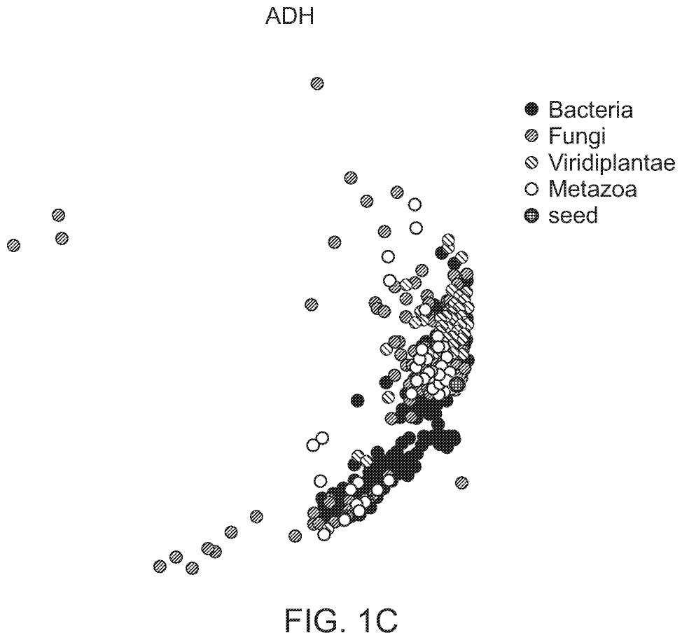

Two complementary approaches were used to design a library for each enzyme family (LeuDH, KivD, and Adh): metagenomic sourcing and rational design (Table 2). For each enzyme, a metagenomic library of >1000 enzymes was designed to sample the full metagenomic sequence space available in sequence databases (FIGS. 1A-1C). For the LeuDH and Adh libraries, available structural data was used for rational design of the B. cereus LeuDH and S. cerevisiae Adh enzymes. Enzyme sequences for all libraries were optimized for expression in E. coli and synthesized in an inducible E. coli expression vector and transformed into E. coli for high throughput screening.

TABLE 2
Enzyme library composition.

| | | | | | | Total |
|---|---|---|---|---|---|---|
| Library | Bacteria | Fungi | Animal | Plant | Rational | Designs |
| LeuDH | 1129 | 11 | 23 | 12 | 270 | 1445 |
| KivD | 783 | 508 | 1 | 4 | 0 | 1296 |
| Adh | 654 | 273 | 128 | 122 | 140 | 1317 |

### Example 2: Characterization of Pathway Enzyme Libraries

### Materials and Methods

### Cell Growth and Enzyme Preparation

For each of the enzyme libraries screened, strains harboring library plasmids were transformed into E. coli T7 expression host cells. 5 μL/well of thawed glycerol stocks were stamped into 500 μL/well of LB+100 ug/mL Carbenicillin (LB-Carb100) in half-height deepwell plates, which were sealed with AeraSeals. Samples were incubated at 37° C. and shaken at 1000 RPM in 80% humidity overnight. 50 μL/well of the resulting precultures were stamped into 450 μL/well of LB-Carb100+1 mM IPTG in half-height deepwell plates, which were sealed with AeraSeals. Samples were incubated at 30° C. and shaken at 1000 RPM in 80% humidity overnight. 250 μL/well of the resulting production cultures were stamped into deepwell plates containing 500 uL of phosphate buffered saline (PBS) and centrifuged for 10 minutes at 4000*G. Supernatant was removed and the resulting cell pellet was resuspended in 200 μL of BugBuster Protein Extraction Reagent+1 μL/mL purified Benzonase+1 μL/6 mL purified Lysozyme. Samples were incubated for 10 minutes at room temperature to generate the cell lysates used in in vitro enzyme assays.

### LeuDH Activity Assay

10 μL of lysate for the LeuDH library strains was transferred to a half-area flat-bottom plate containing 90 μL/well assay buffer (20 mM amino acid [L-Leucine, L-Valine, or L-Isoleucind 200 mM Glycine, 200 mM KCl, 0.4 mM NAD, pH 10.5). Optical measurements were taken on a plate reader, with absorbance readings taken at 340 nm for 10 minutes. The resulting kinetic data was used to resolve the maximum rate of NAD+ reduction, a proxy for LeuDH activity.

### KivD Activity Assay

10 μL of lysate for the KivD library strains was transferred to a half-area flat-bottom plate containing 90 μL/well assay buffer (100 mM PIPES-KOH, 100 mM Potassium glutamate, 1 mM Dithiothreitol, 0.4 mM NAD, 1.5 mM Thiamine pyrophosphate, 10 mM Magnesium glutamate, 20 mM ketoisocaproate (KIC), pH 7.5). A coupling enzyme was used to indirectly measure KivD activity on KIC. Optical absorbance measurements were taken over 10 minutes. The resulting kinetic data was used to determine KivD activity.

### Adh Activity Assay

10 μL of lysate for the Adh library strains was transferred to a half-area flat-bottom plate containing 90 μL/well assay buffer (50 mM MOPS buffer, 0.4 mM NADH, and 30 mM isovaleraldehyde, pH 7.0). Optical absorbance measurements were taken on a plate reader at 340 nm for 10 minutes. The resulting kinetic data was used to resolve the maximum rate of NADH oxidation, a proxy for ADH activity.

### LeuDH Selectivity Assay

To measure LeuDH selectivity (specific deamination of L-Leu in the presence L-Ile and L-Val), lysate was diluted four-fold in lysis buffer, and 10 μL/well of the newly diluted lysate was stamped into 90 μL/well of a modified assay buffer from above, featuring 0.5 mM of each amino acid (L-leucine, L-isoleucine, L-valine), 200 mM Glycine, 200 mM Potassium chloride, and 4 mM NAD. The reaction was quenched at different timepoints and submitted for LC-MS quantification of leucine, isoleucine, and valine.

### Results

To screen the 3×˜1300-member enzyme libraries, high-throughput (HTP) methods were developed to screen for LeuDH, KivD, and Adh enzyme activities in E. coli cell lysates. In brief, strains were cultivated in 96-deepwell plates to induce protein production, with positive and negative control strains included in each plate. Cells were lysed, and enzyme activity was measured in cell lysates using the enzyme-specific spectrophotometric assays described herein. Enzyme assays were executed on a fully automated robotic workcell. For each enzyme family, the full library (˜1300 members each) was measured in biological duplicate, and 50-200 enzymes with the highest activity in each enzyme family were selected as primary “hits” for that family. The primary hits were re-screened in a secondary screen with additional replication (4 biological replicates) to validate the enzyme rankings.

### Leucine Dehydrogenase (LeuDH)

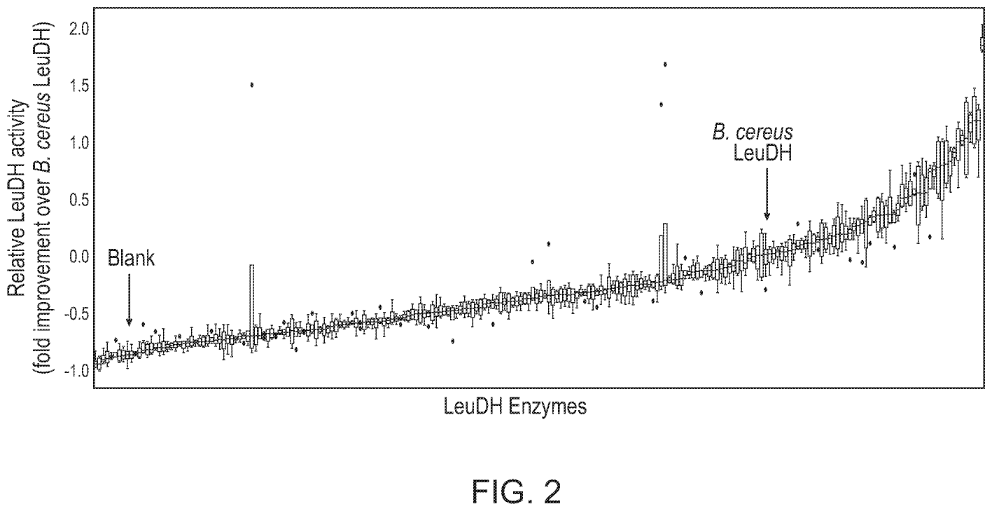

A total of 1378 LeuDH enzymes were first screened for the ability to deaminate Leu. An initial round of screening identified 220 enzymes (Table 4) with activity similar to or better than the parent LeuDH enzyme from B. subtilis. These primary hits were further analyzed in a secondary screen (FIG. 2). In the secondary screen, LeuDH enzymes with up to 1.8-fold increase in LeuDH activity on Leu were validated.

Activity was calculated as: Enzyme Activity divided by Background Enzyme Activity minus 1. Controls were set to 0, and strains with values >0 were considered as potential hits. The value represents a fractional improvement over the control. As a non-limiting example, strains with a 50% improvement would be indicated in Table 4 with a value of 0.5.

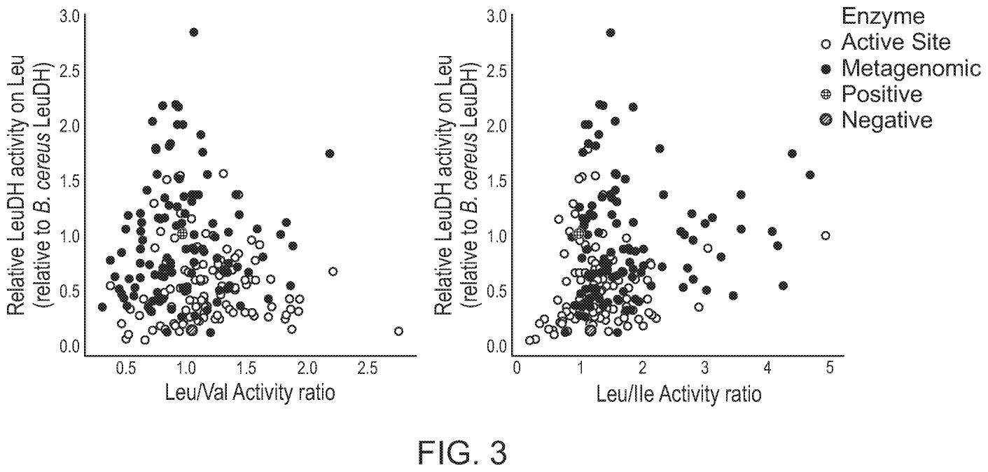

To determine if any of the primary LeuDH hits exhibited increased specificity for Leu over Ile and Val, all 220 primary hits were also screened for activity on Val and Be. Specificity was measured as the ratio of activity on Leu to the activity on Be or Val. As shown in FIG. 3, enzymes that were hits from the primary screen exhibited up to ˜2.7-fold preference for Leu over Val, and up to a 5-fold preference for Leu over Ile. The positive control B. cereus LeuDH showed equal preference for Leu, Val, and Ile when measured in this assay.

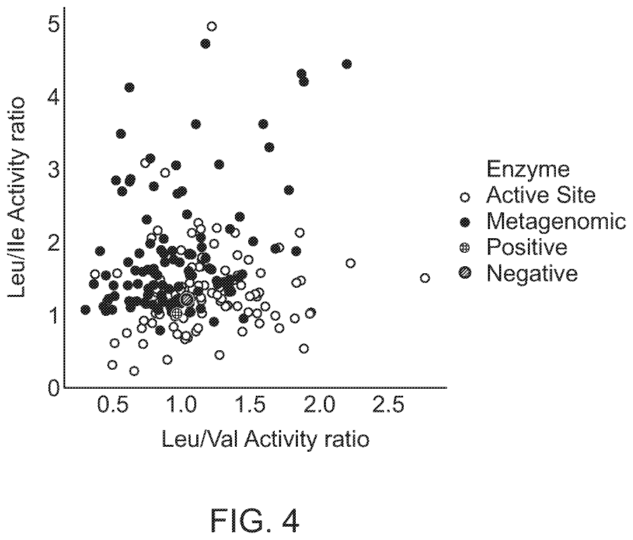

A trade-off of Leu specificity for Leu activity was observed in this library, where the most specific LeuDH enzymes were not the most active LeuDH enzymes. By comparing specificity for Leu/Ile to Leu/Val, hits with increased specificity for Leu relative to both Leu and Val were identified (FIG. 4). The control B. cereus LeuDH exhibited approximately equal preference for Leu, Val, and Ile.

### Ketoisovalerate Decarboxylase (KivD)

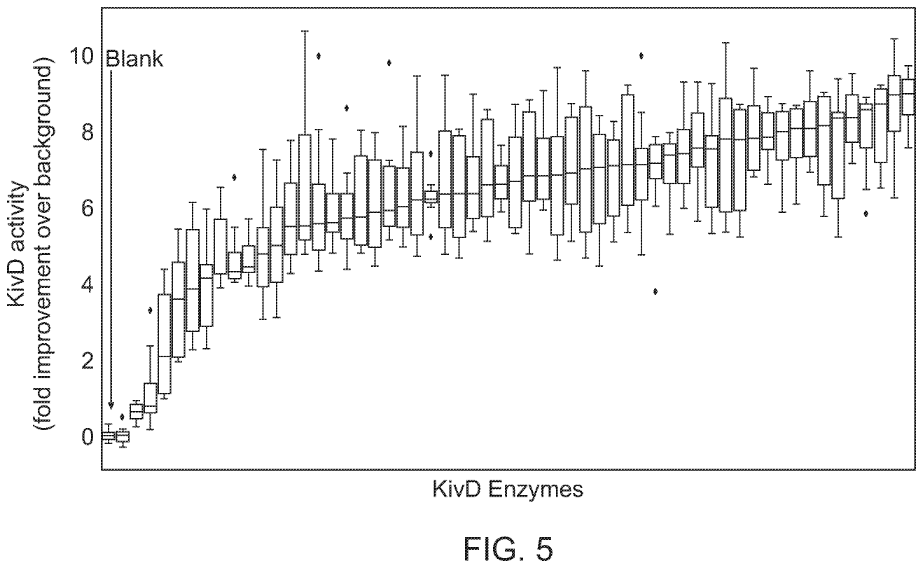

A total of 1248 KivD enzymes were screened for the decarboxylase activity on ketoisocaproate. An initial round of screening identified 55 enzymes (Table 5) with higher activity than the parent KivD enzyme from S. aureus, which did not exhibit activity greater than the background lysate decarboxylase activity in this assay and was equated to the non-zero measurable background activity. These primary KivD hits were further analyzed in a secondary screen (FIG. 5) (Table 5). In the secondary screen, >40 KivD enzymes with at least 6- to 8-fold increase in KivD activity relative to the background lysate activity in this assay were identified. KivD activity was calculated as: Enzyme Activity divided by Background Enzyme Activity minus 1.

### Alcohol Dehydrogenase (Adh)

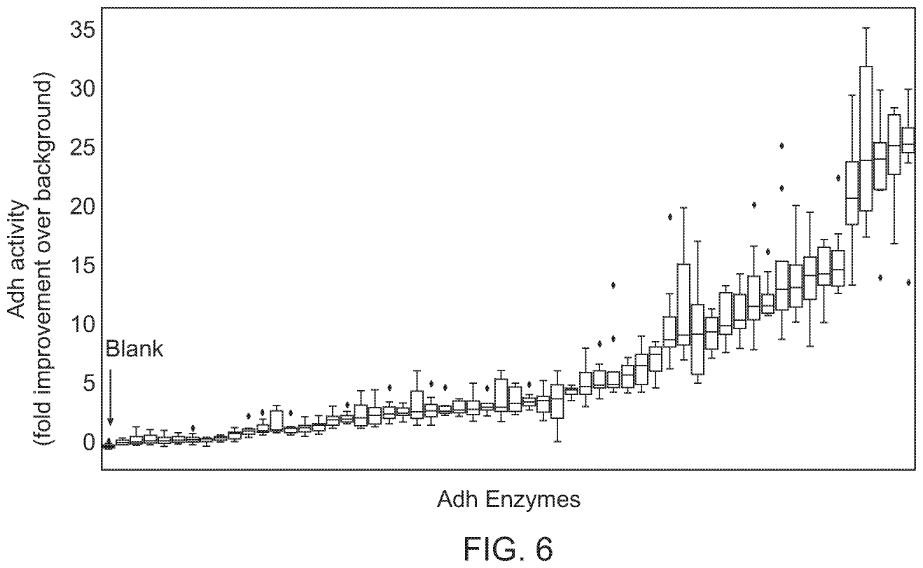

A total of 1215 Adh enzymes were screened for the ability to reduce isovaleraldehyde to isopentanol. An initial round of screening identified 55 enzymes (Table 6) with higher activity than the parent ADH2 enzyme from S. cerevisiae, which did not exhibit activity greater than the background lysate alcohol dehydrogenase activity in this assay and was equated to the non-zero measurable background activity. Because activity of the ADH2 enzyme for S. cerevisiae was indistinguishable from the background activity of the lysate, an Equus caballus Adh with activity higher than the background activity was used as a positive control for the screen. These primary hits were further analyzed in a secondary screen (FIG. 6) (Table 6). In the secondary screen, 5 Adh enzymes with at least 20-fold increase in Adh activity relative to the background lysate activity were identified. The ADH2 enzyme for S. cerevisiae was used as a control for the secondary screen. Adh activity was calculated as: Enzyme Activity divided by Background Enzyme Activity minus 1.

### Example 3: Selectivity of Top LeuDH Candidate Enzymes

### Materials and Methods

### LeuDH Selectivity Assay

To measure LeuDH selectivity (specific deamination of L-Leu in the presence L-Ile and L-Val), lysate was diluted four-fold in lysis buffer, and 10 μL/well of the newly diluted lysate was stamped into 90 μL/well of a modified assay buffer from above, featuring 0.5 mM of each amino acid (L-leucine, L-isoleucine, L-valine), 200 mM Glycine, 200 mM Potassium chloride, and 4 mM NAD. The reaction was quenched at different time points and submitted for LC-MS quantification of leucine, isoleucine, and valine.

### Results

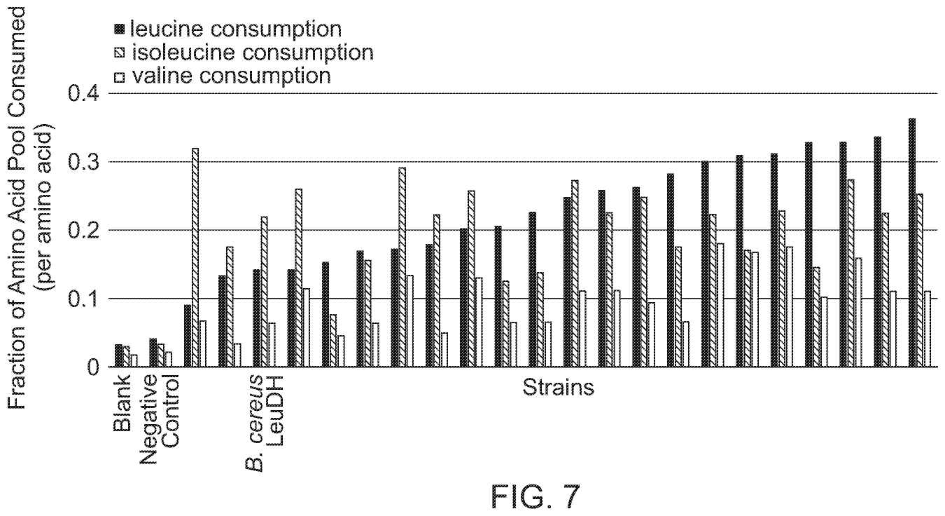

LeuDH catalyzes the deamination of Leu, Val and Be, and as a consequence all substrates have the potential to act as competitors in an in vivo context where substrate pools are mixed. In order to better predict the performance of the top LeuDH hits with regard to mixed-substrate pools, the selectivity of LeuDH enzymes for Leu (i.e., the preference of LeuDH for Leu when Leu, Val, and Ile are all present in the reaction mixture) was measured. A total of 21 LeuDH enzymes were screened in cell lysate assays similar to the HTP screen, except that the reaction mixture contained Leu, Val, and Ile at 1:1:1 molar ratio. Rate of Leu, Val, and Ile disappearance was monitored in the reaction mixture. FIG. 7 shows consumption of Leu, Ile, and Val within the reaction mixture for each LeuDH enzyme. At least 10 LeuDH enzymes showed improved preference for Leu over Val and Be when compared to the parent B. subtilis LeuDH. For nearly all LeuDH enzymes, least preference was shown for valine.

### Example 4: Pathway Enzyme Hit Selection and Operon Assembly

To improve the overall Leu consumption of the BCAA pathway, multiple enzymes for each step that demonstrated superior performance relative to the parent enzyme were selected. For LeuDH, 6 hits were selected based on two criteria: enzyme activity on Leu and specificity for Leu relative to Val and Ile. Because LeuDH selectivity analysis was run in parallel to operon assembly, the selectivity data set did not factor into LeuDH selection. For KivD and ADH, 3 hits were selected for each enzyme family based on in vitro enzyme activity. In total, 12 enzymes were advanced to the final operon design (Table 3). The operon was composed of four coding sequences for enzymes in the following order: LeuDH-KivD-Adh-BrnQ. A preferred operon for Leu consumption was selected and further tested as described below.

TABLE 3
Enzymes selected for advancement to operon design.

| | | | SEQ ID NO | SEQ ID NO |
|---|---|---|---|---|
| Enzyme | Identifier | Source | (Nucleic Acid) | (Amino Acid) |
| LeuDH | t160946 | Cetobacterium ceti | 1 | 2 |
| LeuDH | t160389 | Hymenobacter daecheongensis | 3 | 4 |
| LeuDH | t160283 | Hymenobacter sp. CRA2 | 5 | 6 |
| LeuDH | t160434 | Arenimonas sp SCN 70-307 | 7 | 8 |
| LeuDH | t160048 | Candidatus kapabacteria sp. 59-99 | 9 | 10 |
| LeuDH | t160141 | Peptococcaceae bacterium CEB 3 | 11 | 12 |
| KivD | t163988 | Candida auris | 13 | 14 |
| KivD | t164076 | Bacillus sp. FJ AT-1801 | 15 | 16 |
| KivD | t163842 | Erwinia iniecta | 17 | 18 |
| Adh | t159319 | Tortispora caseinolytica NRRL Y- | 19 | 20 |
| | | 17797 | | |
| Adh | t159028 | Rhizobiales bacterium NRL2 | 21 | 22 |
| Adh | t158538 | Alcanivorax dieselolei | 23 | 24 |

### Example 5: Operon Testing

### Materials and Methods

### Cell Preparation

Branched-chain amino acid (BCAA) pathway operon plasmids were transformed into E. coli Nissle strain 1917, which was purchased from the German Collection of Microorganisms and Cell Cultures (DSMZ Braunschweig, E. coli DSM 6601). Transformed cells were thawed on ice and cell density was measured by light absorption at 600 nm (OD₆₀₀). OD₆₀₀ of 1.0 was assumed to be equal to 10⁹ cells/mL in this method. A volume was calculated to target 1 mL of 2×10⁹ cells/mL cell resuspension, and the cells were transferred into a 96-deep well plate and washed once with cold PBS. After centrifugation (4000 rpm, 4° C., 10 min), the PBS was discarded, and the cell pellets were then resuspended in 1 mL of 1×M9+50 mM MOPS+0.5% glucose (MMG) buffer. Eight hundred (800) μL of each sample was transferred into a new 96-deep well plate and 800 μL of MMG containing 16 mM leucine was added, mixed well by pipetting. A sample (200 μL) assigned as time zero was collected at this moment. The plate was then covered by a breathable membrane and moved to an anaerobic chamber to incubate at 37° C. Samples were also collected at 2 hours and 4 hours during incubation in the anaerobic chamber. The samples were centrifuged for 10 minutes at 4000 rpm at 4° C. immediately after collection. 100 μL of the supernatant was transferred into a new 96-well plate and stored at −80° C. for future analysis.

### Leucine Activity Assay

Leucine was quantitated in bacterial supernatant by liquid chromatography coupled to tandem mass spectrometry (LC-MS/MS) using either an Ultimate 3000 UHPLC-TSQ Quantum or a Vanquish UHPLC-TSQ Altis system. Samples were extracted with 9 parts 2:1 acetonitrile:water containing 1 μg/mL leucine-d3 as an internal standard, vortexed, and centrifuged. Supernatants were diluted with 9 parts 0.1% formic acid and analyzed concurrently with standards processed as above from 0.8 to 1000 μg/mL. Samples were separated on a Phenominex Synergi 4 um Hydro-RP 80A, 75×2 mm using a 0.1% formic acid (A), 0.1% formic acid/acetonitrile (B) at 0.3 mL/min and 50 degrees C. After a 2 μL injection and an initial 5% B hold from 0 to 0.5 minutes, analytes were gradient eluted from 5 to 90% B over 0.5 to 1.5 minutes followed by high organic wash and aqueous equilibration steps. Analytes were detected using Selected Reacting Monitoring (SRM) of compound specific collision induced fragments in electrospray positive ion mode (leucine: 132>86, isoleucine: leucine-d3: 135>89). SRM chromatograms were integrated, and the unknown/internal standard peak area ratios were used to calculate concentrations against the standard curve.

### Results

The top Leu consuming operons identified through HTP screening were transformed into E. coli Nissle 1917 (and labeled as strain 5941, 5942 and 5943) and compared to the prototype strain 1980. Strain 5941 contains the LeuDH enzyme of Cetobacterium ceti, the KivD enzyme of Erwinia iniecta, and the Adh enzyme of Alcanivorax dieselolei. Strain 5942 has the LeuDH enzyme of Cetobacterium ceti, the KivD enzyme of Erwinia iniecta, and the Adh enzyme of Rhizobiales bacterium NRL2. Strain 5943 has LeuDH enzyme of Cetobacterium ceti, the KivD enzyme of Erwinia iniecta, and the Adh enzyme of Rhizobiales bacterium NRL2. The operons further contain BrnQ of E. coli. The prototype strain contains Bacillus cereus LeuDH, Lactococcus lactis KivD, Saccharomyces cerevisiae ADH2, as well as E. coli BrnQ.

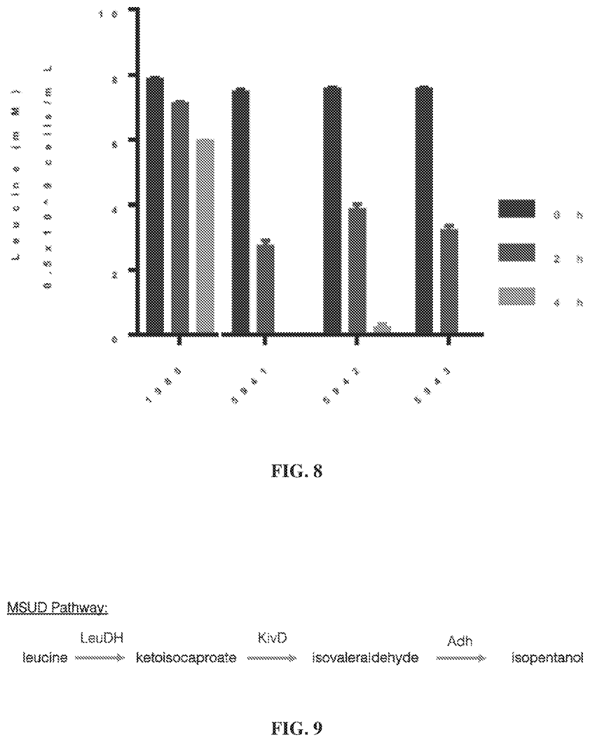

Samples from the top Leu consuming operons and the prototype strain were analyzed for Leu consumption (FIG. 8). The top Leu consuming operon-containing strains (5941, 5942 and 5943) were found to consume Leu at a significantly faster rate than the prototype strain (1980).

### Example 6: Engineering of LeuDH Enzymes and Bioinformatics Analysis of Active LeuDH Enzymes

As shown in Table 4, mutants of UniProt P0A392 (SEQ ID NO: 27) from Bacillus cereus were generated and tested to determine whether the mutants showed improved activity or enzyme expression relative to UniProt P0A392 (SEQ ID NO: 27). The LeuDH activity assay described in Example 2 was used. Point mutations at the following unique positions were observed to improve either activity or enzyme expression: 42, 43, 44, 67, 71, 76, 78, 113, 115, 116, 136, 293, 296, 297, and 300.

The following point mutations in UniProt P0A392 (SEQ ID NO: 27) were observed to improve either activity or protein expression: A115N, A115Q, A115S, A115T, A115V, A297C, A297D, A297E, A297F, A297H, A297K, A297L, A297M, A297N, A297Q, A297R, A297T, A297W, A297Y, E116A, E116L, E116M, E116N, E116R, E116S, E116V, E116W, G43E, G43F, G43T, G43W, G43Y, G44H, G44I, G44K, G44Y, 1113F, 1113M, 1113Q, 1113V, 1113W, 1113Y, L300A, L300C, L300D, L300F, L300H, L300K, L300M, L300N, L300Q, L300R, L300S, L300T, L300W, L300Y, L42A, L42Q, L42T, L76E, L76F, L76H, L761, L76K, L76M, L76R, L76S, L76T, L76W, L76Y, L78C, L78F, L78H, L78K, L78Q, L78V, L78Y, M67A, M67E, M67K, M67Q, M67S, M67T, N71C, N71D, N71H, N71K, N71M, N71T, T136E, T136F, T136L, T136R, T136S, T136Y, V293A, V293C, V293Q, V293S, V293T, V296A, V296C, V296E, V296I, V296K, V296L, V296N, V296S, and V296T.

Bioinformatics analysis was conducted on mutants of SEQ ID NO: 27 and sequences from a metagenomic library that were hits. A list of unique residues found in hits is provided below in Table 7. The corresponding position in SEQ ID NO: 27 is shown. A hit is a LeuDH that has increased activity (greater than 0) relative to SEQ ID NO: 27. For each position in the multiple sequence alignment, individual residue identities were binned into hits and non-hits, and the set difference was calculated. These are residues that are unique to the hit set, either via the systematic point mutation library or the metagenomic sequences.

### Example 7: Bioinformatics Analysis of Active KivD Enzymes

Bioinformatics analysis was conducted on hit KivD enzymes that showed increased activity relative to SEQ ID NO: 29. A list of unique residues found in hits is provided in Table 8. For each position in the multiple sequence alignment, individual residue identities were binned into hits and non-hits, and the set difference was calculated. These are residues that were unique to the hit set. The corresponding position in SEQ ID NO: 29 is indicated in Table 8.

UniProt Q684J7, from Lactococcus lactis, is a microbe widely used in the production of buttermilk and cheese. While not the named reaction for natural enzymes, KivD catalyzes the decarboxylation of 4-methyl-2-oxopentanoate to form isopentanol. It was found that hits from the KivD enzyme library have broadened substrate specificity beyond their natural substrate, which is α-ketoisovalerate.

### Example 8: Bioinformatics Analysis of Active ADH Enzymes

Bioinformatics analysis was conducted on hit ADH enzymes that showed increased activity relative to SEQ ID NO: 31. A list of unique residues found in hits is provided in Table 9. For each position in the multiple sequence alignment, individual residue identities were binned into hits and non-hits, and the set difference was calculated. These are residues that were unique to the hit set. The corresponding position in SEQ ID NO: 31 is indicated in Table 9.

### Example 9: Molar Balance Closure of the Isopentanol Pathway

The performance and molar balance closure of the isopentanol pathway in strain 5941 was assessed in AMBR® 15 bioreactors. Strain 5941 comprises the LeuDH enzyme of SEQ ID NO: 2, the KivD enzyme of SEQ ID NO: 18, and the Adh enzyme of SEQ ID NO: 24. The reactors were filled to 17 mL with M9 media with 0.5% glucose, 10 mM Leu, 10 mM Val, and 5 mM Ile. Conditions were controlled with 0% dissolved oxygen and pH at 7.0. Activated biomass was inoculated to an OD600 of 1, and samples of the supernatant were taken over time to monitor metabolite concentrations.

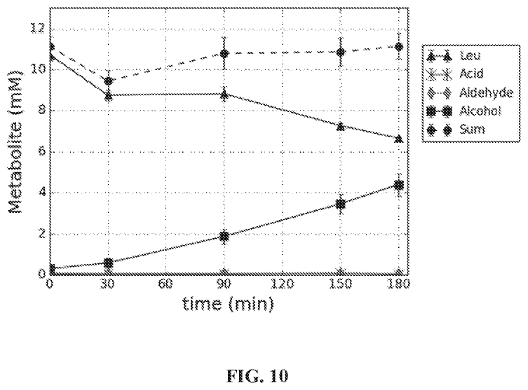

The extracellular concentration profiles of pathway intermediates are shown in FIG. 10. Over the course of 180 minutes, 4.1±0.3 mM of Leucine was consumed and 4.4±0.5 mM of isopentanol accumulated in the media. The keto-acid (2-oxoisocaproate) and aldehyde (isovaleraldehyde) were not observed in the supernatant. Thus, the flux through the pathway is balanced and accounted for. This is also shown by the conservation of total moles of the pathway intermediates (data corresponding to “Sum” in FIG. 10).

### Methods—Fermentation

The assay was performed in an AMBR15f, microbioreactor system from Sartorius. The vessels were filled with 17mls of 1×m9 media salts, supplemented with 2.0 mm MgSO4, 0.1 mM CaCl, 5% glucose, 10 mM L-leucine, 5 mM L-isoleucine, and 10 mM valine. The vessels were filled 18 hrs prior to inoculation, to enable both the pH and DO optodes to hydrate. The temperature in the reactors was kept at 37° C., the pH was maintained at 7 using 2N NaOH, and the dissolved oxygen was kept at 0 using a 0.14vvm N2 flow rate. The agitation was set to 500 RPM to enable good mixing throughout the experiment. The bioreactors were inoculated to an OD600 of 1, from activated biomass supplied by Synlogic. The bioreactors were sampled at 0, 30, 90, 150, and 180 minutes post inoculation. Samples were immediately centrifuged at 15000×g for 30secs in a microcentrifuge and the supernatant was removed for analysis. Supernatants were stored at −20° C. until ready for analysis.

### Methods—Analytics

Analytics were developed for two methods. One method involved liquid chromatography mass spectrometry (LCMS) for the quantification of leucine (Leu), ketoisocaproate acid (Leu acid), and isovaleraldehyde (Leu aldehyde). This method was also validated and used for quantification of valine and isoleucine (and their respective acid and aldehyde products). The second method involved gas chromatography mass spectrometry (GCMS) for the quantification of isopentanol (Leu alcohol). Together, these analytical methods allowed for quantitation of all pathway intermediates for strain 5941. The GCMS method was also validated and used for quantification of valine and isoleucine alcohol products.

LCMS analysis was performed on a Thermo Ultimate 3000 UPLC system with a Thermo Q-Exactive quadrupole-orbitrap mass detector and a Thermo Accucore PFP column (2.1×100 mm, 2.6 μm packing) using the following elution solvents: A=0.1% formic acid and 0.1% TFA in water; B=0.1% formic acid in acetonitrile. The gradient was at 0.5 mL/min of 1% B in A for 60 seconds, followed by a linear ramp from 1% to 40% B in A over 270 seconds. The column was then flushed with 95% B in A for 60 seconds, and re-equilibrated with 1% B in A for 180 seconds. MS acquisition was from 0.8 to 5.3 minutes.

Column effluent was introduced into the mass spectrometer via a standard Thermo ESI source with positive mode ionization at +3800V, vaporizer temperature of 400° C., and ion transfer tube temperature of 375° C. Thermo reports gas flow rates in arbitrary units probably approximating L/min at STP. Set points were: sheath gas, 60; aux gas, 30; sweep gas, 1. To increase data acquisition rate, orbitrap resolution was set to 17,500. Quadrupole resolution was 1 m/z.

This method also derivatizes both aldehydes and keto acids, improving the stability of those analytes. Numerous derivatizing agents were explored, and it was found that 2-(Dimethylamino)ethylhydrazine in methanol resulted in the best sensitivity in positive mode. A buffer of 0.5M acetic acid and 0.5M sodium acetate in methanol was used for the quantification of LEU ACID and LEU ALDEHYDE, while also measuring non-derivatized LEU.

GC-MS analysis was performed on an Agilent GCMS/MSD with a Gerstel autosampler, using a J&W DB-WAX GC Column (15m) and chloroform as the extraction solvent. Front injector was set at 250° C. and a flow rate of 1 mL/min. The oven temperature held at 40° C. for 1 minute, followed by a ramp to 130° C. (15° C./min), and then ramped up to 200° C. (65° C./min). Ms acquisition scan window was at 40-150 mz, with the MS source and MS quad at 250C and 200C respectively.

To facilitate high throughput and automation, a Gerstel autosampler was used to inject the extracted bottom chloroform layer in a 96 well plate format with the aqueous ambr15 culture matrix on top acting as an overlay to prevent product evaporation. To account for any other potential alcohol product evaporation, 2-heptanol was added to the chloroform as an internal.

Sequences for Enzymes in Table 3

LeuDH (Identifier: t160946; Accession: A0A1T4PGG9)
ATGAACATCTTCAAGAAAATGGAGGAATTTAATTATGAACAACTGGTCTACTTCTACGACAGCGAAACGGAACTC
AAAGGTATTACCTGTATACACAACACAACTTTAGGGCCGGCATTGGGCGGTACCCGCCTTTGGAACTATAACTCT
GAGGAAGATGCCGTTGAAGACGTAATCCGTCTGGCTCGGGGCATGACTTACAAAGCGGCTTGCGCCGGTCTGAAT
CTGGGCGGCGGTAAAACCGTGCTGATCGGTGATGCTAAAAAGATTAAATCAGAGTCCTACTTCCGTGGACTGGGG
CGCTACGTTCAGTCGCTGAACGGCAGATATATCACCGCGGAAGACGTAAATACTTCTACGAAGGATATGGCATAC
GTTGCTATGGAAACTGACTATGTGGTAGGCCTGGGAGGTAAATCCGGCAACCCTAGTCCAGTTACTGCTTACGGT
GCATTTATGGGTATCAAAGCGGCGCTGATGAAAAAATTTGAGGATAGCTCTATTGAAGGCCGAACCTTCGCAGTG
CAGGGTGCTGGGCAGACGGGTTACTATCTTATCGATTACCTCCTAGGCAACAACAAGTTCAAAGAAAAGGCTAAA
AAAATTTACTTCACCGAAATTAACGAGAGCTATATCGAGCGTATGAACAAAGAACATCCGGAAGTTGAATTTATT
TCCCCGGACAAAATCTACTCGCTGGAAGTAGACGTCTTCGTGCCCTGCGCCCTGGGCAAAATCGTTAATGACAAA
ACTATCGATGAATTTAAGTGTCCGATCATCGCAGGTACTGCAAACAACGTACTGGAAAGGGAAGCGCACGGCAAC
ATGCTTAAAGAACGTGGCATTCTTTACGCCCCGGACTATGTGATCAATGCTGGTGGGCTGATCAACGTTTACCAC
GAGCTGAACGGTTACAATAAAGAGAACGCTATTCTGGAAGTGGAATTAATTTATGATCGCCTACTGGAAATATTC
AACATCGCTGATTCTCTGAACATCAGCACCAATATCGCTGCCAACGAGTTCGCGGAAAAACGTATCAAGCAAATT
AAGTCCTTGAAAAACAACTTCATTAAACGC (SEQ ID NO: 1)
MNIFKKMEEFNYEQLVYFYDSETELKGITCIHNTTLGPALGGTRLWNYNSEEDAVEDVIRLARGMTYKAACAGLN
LGGGKTVLIGDAKKIKSESYFRGLGRYVQSLNGRYITAEDVNTSTKDMAYVAMETDYVVGLGGKSGNPSPVTAYG
AFMGIKAALMKKFEDSSIEGRTFAVQGAGQTGYYLIDYLLGNNKFKEKAKKIYFTEINESYIERMNKEHPEVEFI
SPDKIYSLEVDVFVPCALGKIVNDKTIDEFKCPIIAGTANNVLEREAHGNMLKERGILYAPDYVINAGGLINVYH
ELNGYNKENAILEVELIYDRLLEIFNIADSLNISTNIAANEFAEKRIKQIKSLKNNFIKR (SEQ ID NO: 2)
LeuDH (Identifier: t160389; Accession: A0A1M6BE59)
ATGGTAGAGATCAAGGCTTTGACGGACACTTCCGTGTTTGGGCAAATTGCAGAACACCAGCATGAACAGGTCGTT
TTCTGCCACGATCACGAAACCGGCCTCCGTGCGATCATCGGTATTCATAACACAGTTCTTGGCCCCGCCTTAGGT
GGAACTCGCATGTGGCACTATGCTTCTGACGCAGAGGCGCTGAATGATGTTCTGCGTCTGTCGCGCGGTATGACC
TACAAAGCTGCTATAAGTGGCCTGAACCTGGGTGGCGGTAAAGCAGTGATCATTGGGGACGCCAAAACCCTGAAA
ACCGAAGCGCTGCTGCGGAAGTTCGGCAGATTCGTAAAAAACCTGAATGGTAAATACATCACTGCTGAAGATGTC
AACATGACTACAAAAGACATGGAGTACATCAGGATGGAAACCAAGCACGTTGCTGGCTTACCTGAATCAATGGGT
GGAAGCGGTGATCCGTCCCCGGTGACTGCATTTGGTACGTATATGGGCATGAAAGCGGCGGCCAAAAAAGCGTTC
GGCTCTGACTCTCTGGCTGGCAAACGTATCGCTGTTCAGGGTGTAGGTCATGTCGGCACTTACCTGTTGGAGTAT
TTGCAGAAGGAAGGTGCTAAGCTGGTACTGACTGACTACTATGAAGATCGTGCCCTGGAGGCAGCAACGCGTTTT
GGCGCAAAAATGGTTGGCCTGGACGAAATTTACGATCAAGACGTTGATATCTACAGTCCATGTGCTCTTGGAGCT
ACCATTAACGATGACACTATCGGTCGCCTGAAATGCCAGGTTATCGCTGGTTGCGCAAACAACCAGCTGCAAAAC
GAAAATGTGCATGGCCCGGCCCTCGTGGAGCGCGGGATTGTGTACGCTCCGGATTTCCTGATCAACGCCGGCGGC
CTGATCAACGTTTACTCGGAAGTAGTGGGTAGCTCCCGTCAGGGTGCTTTGAACCAGACCGAAAAAATTTTCGAC
ATCACCACTCAGGTTCTAAACAAAGCGGAACAAGAGGGTTCTCACCCGCAGGCGGCAGCTACTAAGCAGGCTGAA
GAGCGTATTGCAAGCCTGGGCAAAGTTAAGAGCACCTAC(SEQ ID NO: 3)
MVEIKALTDTSVFGQIAEHQHEQVVFCHDHETGLRAIIGIHNTVLGPALGGTRMWHYASDAEALNDVLRLSRGMT
YKAAISGLNLGGGKAVIIGDAKTLKTEALLRKFGRFVKNLNGKYITAEDVNMTTKDMEYIRMETKHVAGLPESMG
GSGDPSPVTAFGTYMGMKAAAKKAFGSDSLAGKRIAVQGVGHVGTYLLEYLQKEGAKLVLTDYYEDRALEAATRF
GAKMVGLDEIYDQDVDIYSPCALGATINDDTIGRLKCQVIAGCANNQLQNENVHGPALVERGIVYAPDFLINAGG
LINVYSEVVGSSRQGALNQTEKIFDITTQVLNKAEQEGSHPQAAATKQAEERIASLGKVKSTY
(SEQ ID NO: 4)
LeuDH (Identifier: t160283; Accession: A0A1S9B636)
ATGGTAGAGATCCAGGCTTTGCCGGAAACTTCCATTTTTGGGCAAATCGCAGACCACCAGCATGAACAGGTGGTC
TTCTGCCACGATCACGAAACCGGCCTCCGTGCGATAATCGGTATTCATAACACGGTTCTTGGCCCCGCCTTAGGT
GGAACTCGCATGTGGCACTATGCTACCGAGGCAGAAGCGCTGAATGACGTTCTGCGTCTGTCTCGCGGTATGACC
TACAAGGCTGCTATCTCGGGCCTGAACCTGGGTGGCGGTAAAGCAGTAATCATTGGGGATGCCAAAACAATCAAA
ACCGAAGCGCTGCTGCGGAAATTCGGCAGATTCGTGCAGAACCTGAATGGTAAATACATCACTGCTGAAGACGTT
AACATGACTACAAAGGATATGGAGTACATTAGGATGGAAACCAAACACGTCGCTGGCTTACCTGAAAGTATGGGT
GGAAGCGGTGACCCGTCACCGGTAACTGCATATGGTACGTACATGGGCATGAAAGCGGCGGCCAAAAAGGCGTTT
GGCTCTGATTCCCTGGCTGGCAAACGTATCGCTGTTCAAGGTGTGGGTCATGTTGGCACTTATCTGCTTGAGCAT
TTGACCAAAGAAGGTGCTCAGATTGTGCTGACTGACTACTATAAGGAACGTGCCGAGGAAGCAGGCGCGCGTTTT
GGCGCACAGGTTGTTGGCCTGGACGATATCTACGATCAAGAGGTCGACATTTACTCTCCATGTGCTCTCGGTGCT
ACCATCAACGATGACACTATCGATCGCCTGCGTTGCGCTGTTGTAGCCGGTTGCGCAAACAACCAGCTGAAAGAA
GAAAACGTCCACGGTCCGGCGCTGGTTGAGCGCGGGATAGTATACGCCCCAGACTTCCTGATCAATGCAGGTGGC
CTGATTAACGTGTATAGCGAAGTTACAGGGTCTACCCGTCAGGGGGCTTTAACTCAGACCGAAAAAATCTATGAC
TACACACTCCAAGTTCTGGAAAAAGCCGCGGCTGAAGGTCTGCACCCGCAGCAGGCTGCGATCCGTCAGGCGGAA
CAACGCATCGCTGCAATTGGTAAGGTGAAAAGCACCTAC (SEQ ID NO: 5)
MVEIQALPETSIFGQIADHQHEQVVFCHDHETGLRAIIGIHNTVLGPALGGTRMWHYATEAEALNDVLRLSRGMT
YKAAISGLNLGGGKAVIIGDAKTIKTEALLRKFGRFVQNLNGKYITAEDVNMTTKDMEYIRMETKHVAGLPESMG
GSGDPSPVTAYGTYMGMKAAAKKAFGSDSLAGKRIAVQGVGHVGTYLLEHLTKEGAQIVLTDYYKERAEEAGARF
GAQVVGLDDIYDQEVDIYSPCALGATINDDTIDRLRCAVVAGCANNQLKEENVHGPALVERGIVYAPDFLINAGG
LINVYSEVTGSTRQGALTQTEKIYDYTLQVLEKAAAEGLHPQQAAIRQAEQRIAAIGKVKSTY
(SEQ ID NO: 6)
LeuDH (Identifier: t160434; Accession: A0A1D2RXB2)
ATGATCTTCGAGACAATTTCTACGTCGAATCACGAAGAAGTTGTGTATTGCCATAACAAGGACGCCGGCTTGAAA
GCAATCATCGCGATTCACAACACTGTACTCGGTCCGGCTCTGGGTGGCACTCGCATGTGGCCCTACGCTAGCGAA
GAGGAAGCACTGAAAGATGTCCTTCGTTTATCCCGTGGGATGACCTACAAAGCTGCGGTTTCAGGTCTAAACCTG
GGCGGCGGTAAAGCTGTGATCTGGGGTGATCCGAATAAAGACAAGTCTGAAGCGCTGTTTAGAGCCTTCGGACGG
TTTGTAAACAGCCTGGGCGGACGCTACATTACCGCGGAGGACGTTGGCATTGATGTTAACGACATGGAATATGTG
CTGCGTGAAACTGATTACGTCACCGGTGTACATCAGGTTCACGGTGGGAGTGGTGATCCTTCTCCATTCACCGCA
TATGGCACTCTGCAAGGCCTGATGGCCGCTCTGCAAGTGAAATTCGGTAACGAAGACGTAGGCAATTACAGCTAC
GCTGTTCAGGGTGTGGGTCACGTTGGCATGGAATTTGTTAAACTGCTGCGTGAGCGCGGTGCAAAGGTTTTCGTC
ACTGACATCAACAAAGATGCGGTCCAGCGTGCTGTGGACGAATTTGGTTGTGAGGCAGTAGCCCTGGATGAAATC
TATGACGTTGATTGCGACGTGTACTCCCCGACCGCTCTGGGCGGCACCGTGAACGATAAAACTTTACCGCGTCTG
AAATGTAAGGTAATCTGCGGTGCGGCAAACAACCAGTTAGCTAATGATGAGATAGGCGTGGAACTGGAAAAAAAA
GGCATCCTCTATGCTCCGGACTACGCGGTCAACGCGGGTGGGCTGATGAACGTTAGCCTGGAAATCGATGGATAC
AACCGCGAACGTGCGATGCGTATGATGCGTACCATTTATTACAATTTGGGTCGCATTTTCGAAATCTCTAAGCGC
GACGGCATCCCTACATTCCGAGCCGCCGATCGTATGGCTGAAGAACGCATAACGGCCATCGGTAAACTGCGTTTA
CCGCATTTGGGCGCTGCGGCACCGCGCTTCCAGGGCCGACGTGGCAAC (SEQ ID NO: 7)
MIFETISTSNHEEVVYCHNKDAGLKAIIAIHNTVLGPALGGTRMWPYASEEEALKDVLRLSRGMTYKAAVSGLNL
GGGKAVIWGDPNKDKSEALFRAFGRFVNSLGGRYITAEDVGIDVNDMEYVLRETDYVTGVHQVHGGSGDPSPFTA
YGTLQGLMAALQVKFGNEDVGNYSYAVQGVGHVGMEFVKLLRERGAKVFVTDINKDAVQRAVDEFGCEAVALDEI
YDVDCDVYSPTALGGTVNDKTLPRLKCKVICGAANNQLANDEIGVELEKKGILYAPDYAVNAGGLMNVSLEIDGY
NRERAMRMMRTIYYNLGRIFEISKRDGIPTFRAADRMAEERITAIGKLRLPHLGAAAPRFQGRRGN
(SEQ ID NO: 8)
LeuDH (Identifier: t160048)
ATGCAGATCTTCGACACTTTGCAATCAATGGGCCATGAGCAGGTGGTCCTATGTAGCGATAAGACCACGGGTCTG
CGCGCCATTATCGCTATACACGATACATCCTTAGGGCCGGCGCTTGGTGGTACCCGTATGTGGCAGTATGCAACT
GACGACGATGCTATTACTGACGCACTCCGTCTGTCTCGGGGCATGACCTACAAAGCTGCGGTTTCTGGCGTAAAT
CTGGGCGGTGGTAAAGCCGTTATCATCGGAAACCCTCACAGTGATAAAAGCGAAGCGCTGTTTCGCGCTTACGGC
AGAATGGTGGAATCCCAGCGTGGGCGTTACATCACCGCCGAAGACGTTGGTACTAGCGTACGTGATATGGAGTGG
ATTCGCATGGAAACCAAATATGTAACGGGCGTGGGTGGCAACGGAGGCTCTGGTGACCCCTCTCCAGTTACCGCT
CTGGGTGTTTACTCGGGCATGAAGGCATGCGCTAAATCAGTCTATGGTACTGATGCGCTGAGCGGTAAAAGGATC
GTGGTTCAGGGCGCGGGTAACGTTGCATCCCATCTGGTTCACAGTCTGGTAAAAGAAGGCGCTGTGGTTTTCGTC
ACTGACATCTACGAAGAAAAGGCCAAAGCATTAGCGGCTGAAACGGGCGCTACCGTGATTCGCACCGACGAGGTT
TTTACTACACAATGCGATATCTTCTCTCCGAACGCTCTGGGGGCCGTCCTGAACGATGAAACTATTCCGCAGCTC
ACATGCGCTATCGTAGCTGGTGGTGCAAACAATCAGCTTAAAATCGAACAACGTCACGCCACGGCTCTGCAAGAG
AAAGGCATTCTGTATGCGCCGGATTACGTAATCAACGCCGGGGGCCTCATGAATGTGGCGAGCGAAGTTGACGGC
TACAACCGTGAAAAGGTTATGCGCCAGGCTGAAGGTATTTACGATATTACTATGAACATCCTAAATACCGCGCGT
GAGCGTAACATCCTGACCATCGAAGCATCCAACGCGATTGCTGAAGAGCGGATCAACAAAGTTCGCCATGTTCAC
GGGAACTTCATCGGTTCCCCGTCTATTCGCGGAGTA (SEQ ID NO: 9)
MQIFDTLQSMGHEQVVLCSDKTTGLRAIIAIHDTSLGPALGGTRMWQYATDDDAITDALRLSRGMTYKAAVSGVN
LGGGKAVIIGNPHSDKSEALFRAYGRMVESQRGRYITAEDVGTSVRDMEWIRMETKYVTGVGGNGGSGDPSPVTA
LGVYSGMKACAKSVYGTDALSGKRIVVQGAGNVASHLVHSLVKEGAVVFVTDIYEEKAKALAAETGATVIRTDEV
FTTQCDIFSPNALGAVLNDETIPQLTCAIVAGGANNQLKIEQRHATALQEKGILYAPDYVINAGGLMNVASEVDG
YNREKVMRQAEGIYDITMNILNTARERNILTIEASNAIAEERINKVRHVHGNFIGSPSIRGV
(SEQ ID NO: 10)
LeuDH (Identifier: tl60141; Accession: A0A0J1FEE3)
ATGACAACGTTCGAGTATATGGAAAAGTACGACTACGAACAACTGGTCCTTTGTCAGGATAACACTTCTGGCCTC
AAAGCAGTAATTTGCATCCATGACACCACTCTGGGGCCAGCTTTGGGTGGCACCCGTATGTGGAATTACGCCAGT
GAAGAAGATGCTATCCTGGATGCGTTACGCCTGGCGCGAGGTATGACTTATAAAAACGCTGCCGCAGGTCTGAAC
CTGGGCGGCGGTAAAGCTGTTATTATGGGCGACAGCCGTACCCAGAAATCAGAGGAACTGTTTCGCGCGTTCGGT
CGTTACGTGCAGGCGCTGAACGGCCGTTATATCACCGCTGAGGACGTTGGTACTAACGTACAAGATATGGACTGG
ATACACATGGAAACAAAGTTTGTGACCGGGATCTCCTCTTCGTACGGTGCTAGCGGAGATCCGTCCCCTCTGACC
GCACTGGGCGTTTACCGCGGTATGAAAGCCGCCGCAAAAGAAGCGTTCGGCAGCGACTCTTTAGAGGGTAAAACT
GTTGCTATTCAGGGTCTTGGCCACGTCGGCTATTACCTGGCAAAACACCTCACTGATGAAGGCGCTAAACTGATC
GTGACGGATATCAATTCTGAAGCCGTTAAGAGGGTAGCGCGTGAGTTCGTTGCTACCGCAGTCCGTACCGAAGAA
ATTTTCGGCGTTAAATGCGACATCTTTGCGCCCTGTGCTCTGGGTGCAGTTATCAACGATGAAACCATTCCGCAG
CTGAAGTGCCAGGTAGTTGCCGGTGCTGCGAACAATGTGTTGAAAGAGGATCGCCATGGTGACGAACTATACGAA
AAAGGAATCCTGTACGCTCCGGACTATGTAATTAACGCGGGCGGCGTTATCAACGTGGCCGACGAACTGGAAGGT
TACAACGCTGAACGTGCTCTGAAAAAAGTTGAGATGGTATATGATAATGTGGCACGCGTCATCGCTATTGCCAAG
CGTGACCATATCCCGACTTATAAAGCAGCGGACCGAATGGCTGAGGAACGTATTGCGAAAATTGGCAAAGTTTCC
AACACTTTCCTGCGC (SEQ ID NO: 11)
MTTFEYMEKYDYEQLVLCQDNTSGLKAVICIHDTTLGPALGGTRMWNYASEEDAILDALRLARGMTYKNAAAGLN
LGGGKAVIMGDSRTQKSEELFRAFGRYVQALNGRYITAEDVGTNVQDMDWIHMETKFVTGISSSYGASGDPSPLT
ALGVYRGMKAAAKEAFGSDSLEGKTVAIQGLGHVGYYLAKHLTDEGAKLIVTDINSEAVKRVAREFVATAVRTEE
IFGVKCDIFAPCALGAVINDETIPQLKCQVVAGAANNVLKEDRHGDELYEKGILYAPDYVINAGGVINVADELEG
YNAERALKKVEMVYDNVARVIAIAKRDHIPTYKAADRMAEERIAKIGKVSNTFLR (SEQ ID NO: 12)
KivD (Identifier: tl63988; Accession: A0A0L0P8D8)
ATGTCGGAGATCACATTGGGTAGATACCTTTTCGAACGCTTAAACCAACTGCAAGTGCAGACTATTTTTGGGCTG
CCCGGCGACTTCAATCTGTCCCTGCTGGATAAGATCTATGAAGTTGATGGCATGCGTTGGGCAGGTAACGCTAAC
GAACTCAACGCCGCTTACGCGGCTGACGGTTATAGCCGTGTCAAAGGCCTCGCATGTCTGGTTACCACTTTTGGT
GTAGGCGAGCTAAGTGCGCTGAATGGTGTGGGTGGCGCTTACGCAGAACACGTTGGGCTGCTGCATGTAGTGGGC
GTCCCATCAATCTCTAGCCAGGCGAAACAGCTGCTGCTGCACCATACCCTGGGTAACGGAGATTTCACGGTTTTC
CACCGCATGTCCAACAACATTTCTCAGACCACGGCTTTTATCAGCGACATTAATTCTGCTCCTGGTGAAATCGAT
AGGTGCATCCGTGAGGCCTGGGTACATCAGCGTCCGGTTTACGTCGGCCTGCCGGCGAACCTAGTTGACCTGACT
GTGCCGGCGTCTCTGTTAGACACTCCGATCGATCTGTCCTTGAAAAAAAACGACCCGGATGCCCAGGAAGAAGTT
ATTGAAACCGTCCTTGATCTGGTAGACAAGTCTAAAAACCCTATAATCTTAGTTGACGCATGCGCTAGCCGTCAC
TCATGCCGCGATGAAGTACGCCGGTTGGTGGACTCCACCAGCTTCCCGGTTTTCGTTACTCCAATGGGTAAATCT
GCTGTAAATGAGAGTCACCCGCGTTTTGGCGGTGTTTACGTGGGCAGCCTCAGCGAGCCAAACGTAAAAGAAGCC
GTTGAAAACGCTGACCTGGTGCTGTCCATAGGCGCCCTGTTGAGCGACTTCAACACTGGATCGTTCTCTTATTCC
TACAAAACTAAGAACATTGTTGAATTTCACTCTGATTATACCAAAATCCGTCAAGCAACGTTCCCGGGTGTTCAG
ATGAAAGAAGCACTGAATGTCCTGTTGGAAAAAATCCCGAGCCATGTCGCTAACTACAAACCTCTGCCGGTTCCG
CAGCGTCGCGTTATTCCGAGCCCAGGGGATAAGGCTGCGATCTCTCAGGAGTGGCTGTGGTCGCGTCTGTCTAGC
TGGTTCCGCGAGGGCGACATCGTCATTACAGAAACCGGTACCAGTGCGTTTGGAATTGTACAGTCCTATTTCCCA
GATAACTGCATCGGCATCAGTCAGGTGCTGTGGGGTTCGATCGGCTTCACCGTAGGTGCAACGCTGGGCGCGGTG
ATGGCTGCACAAGAAATCGATCCGAAAAAACGTGTGATTTTATTTGTCGGTGACGGTTCTCTGCAACTTACTGTA
CAGGAAATTTCTACCATGGTTAAGTGGGAAACCACTCCCTACCTGTTTGTGCTGAACAACGATGGGTACACTATC
GAACGCCTTATCCATGGCGAGACTGCTACGTATAACGATATTCAGCCGTGGGATAATCTGGGTCTGTTGCCGCTG
TTCAAAGCTCGTGACTACGAAACCAACCGAGTTGCGACTGTAGGCGAAATTGAAGCGCTATTCAACAATTCAGCT
TTCAATGAGAATACAAAGATCCGTATGGTGGAGGTCATGCTGCCGCGGATGGATGCACCACAGAACCTGGTTAAA
CAGGCTGAATTTTCCTCCAAGACCAACAGCGAAAAC(SEQ ID NO: 13)
MSEITLGRYLFERLNQLQVQTIFGLPGDFNLSLLDKIYEVDGMRWAGNANELNAAYAADGYSRVKGLACLVTTFG
VGELSALNGVGGAYAEHVGLLHVVGVPSISSQAKQLLLHHTLGNGDFTVFHRMSNNISQTTAFISDINSAPGEID
RCIREAWVHQRPVYVGLPANLVDLTVPASLLDTPIDLSLKKNDPDAQEEVIETVLDLVDKSKNPIILVDACASRH
SCRDEVRRLVDSTSFPVFVTPMGKSAVNESHPRFGGVYVGSLSEPNVKEAVENADLVLSIGALLSDFNTGSFSYS
YKTKNIVEFHSDYTKIRQATFPGVQMKEALNVLLEKIPSHVANYKPLPVPQRRVIPSPGDKAAISQEWLWSRLSS
WFREGDIVITETGTSAFGIVQSYFPDNCIGISQVLWGSIGFTVGATLGAVMAAQEIDPKKRVILFVGDGSLQLTV
QEISTMVKWETTPYLFVLNNDGYTIERLIHGETATYNDIQPWDNLGLLPLFKARDYETNRVATVGEIEALFNNSA
FNENTKIRMVEVMLPRMDAPQNLVKQAEFSSKTNSEN (SEQ ID NO: 14)
KivD (Identifier: tl64076; Accession: A0A0M5JJZ2)
ATGACAAGCATGGACAATTCTAGTCAGCAAATCCCCATGGGTCAGAAAACCGTCGGGGAGTACTTGTTCGATTGC
CTCAAGCAGGAAGGCATAACGGAAATCTTTGGTGTGCCGGGCGATTATAACTTCACCTTACTGGACGCCCTGCAA
GAATACAACGGTATTCGTTTCTATAACGGCCGCAACGAGCTGAATGCTGGCTACGCAGCTGACGGTTACGCGCGT
ATTAAAGGAATCTCCGCGCTAATCACTACTTTTGGTGTTGGTGAACTGTCAGCAACTAACGCTATTGCCGGCGCG
AACAGCGAACACGTACCTATCATCCATATTGTTGGGTCCCCACCGGAAAAAGCTCAGAAGGAGCGCAAACTGATG
CACCATACCCTGATGGATGGCAACTTCGACGTATTCCGTAAAGTTTACGAACCGCTTACCGCTTATACTACCATC
GTCACGGCAGATAACGCGCGGATGGAGATCCCGGCTGCTATCCGTATTGCCAAAGAACGAAGAAAGCCAGTGTAC
CTGGTTGTTGCGGATGACGTAGTGGCTAAACCGATTACTGGTCGTGAAGTCCCGGCATCTCCTCTGCCGGCTAGC
AATCAGGACAAACTGCTTGCTGCGGTTGAGCACGTTAGGCGTCTTCTGGAACCTGCACGCCAGCCGGTAATATTG
GTTGATGTGAAAGCCATGCGCTTTGGATTACAGACCGCCGTCAGGGAACTGGCAAACACTATGAATGTTCCAGTG
GCTACAATGATGTATGGCAAAGGCACTTTCGACGAAACCCATCCAAACTACATCGGCGTATATGCGGGTACGTTC
GGTTCGTCTGAAGTTCAATCTATCGTAGAAAACTCGGACTGTGTTATCGCCGTTGGTTTGGTGTGGAGCGATACT
AACACCGCAAACTTTACTGCGAAATTAAACCCGCACAATACCATTGAGGTTCAGCCGACAAAAGTGAAAATCGCT
GAGTCCCAGTACCCCGATGTCCGTGCCGCAGACATCCTGCAAGAAATGCAGAAGCTGGATTATCGTAGCCAGTCT
AAACCGGAAAAAATCTCATTTCCGTACGAAGAGATAACCGGGTCCAGTGATGAACCGCTCCGCGCAGAAAACTAC
TTCCCTCGTTTTCAGCGCATGCTGAAGGAAAACGATATTGTTATCGCTGAGACCGGCACGTTCTACTACGGTATG
AGTCAAGTTAAACTGCCCGCGAACACTACGTACATCATGCAGGGCGGCTGGCAGAGCATTGGTTATGCCACCCCG
GCGGCATACGGCGCGTCTATCGCTGCTCCGGACCGTCGCGTCTTACTGTTCACTGGTGATGGCTCCATGCAGCTG
ACCGCACAGGAAATCTCTTCTATGCTTTATTACGGTTGCAAGCCGATTATCTTTGTACTGAACAATGACGGGTAC
ACCATTGAGCGGTATCTGAATGTAGAAATCTCCCCTGACGAACAAAACTATAACGATATTCCGAACTGGTCTTAT
ACTAAACTGGCTGAGGCGTTCGGTGGTGAACTGTTCACTAAAACAGTGCGTACCAATGAAGAATTGGATGAAGCG
ATCACACAGGCTGAGCAAGAGTACGCCGAAAAACTGTGCCTGATCGAGATGATTGCTGCTGATCCAATGGACGCA
CCGGAATACATGCACCGTATCCGTAACCATAAGCAGGAACAGAAAAAG (SEQ ID NO: 15)
MTSMDNSSQQIPMGQKTVGEYLFDCLKQEGITEIFGVPGDYNFTLLDALQEYNGIRFYNGRNELNAGYAADGYAR
IKGISALITTFGVGELSATNAIAGANSEHVPIIHIVGSPPEKAQKERKLMHHTLMDGNFDVFRKVYEPLTAYTTI
VTADNARMEIPAAIRIAKERRKPVYLVVADDVVAKPITGREVPASPLPASNQDKLLAAVEHVRRLLEPARQPVIL
VDVKAMRFGLQTAVRELANTMNVPVATMMYGKGTFDETHPNYIGVYAGTFGSSEVQSIVENSDCVIAVGLVWSDT
NTANFTAKLNPHNTIEVQPTKVKIAESQYPDVRAADILQEMQKLDYRSQSKPEKISFPYEEITGSSDEPLRAENY
FPRFQRMLKENDIVIAETGTFYYGMSQVKLPANTTYIMQGGWQSIGYATPAAYGASIAAPDRRVLLFTGDGSMQL
TAQEISSMLYYGCKPIIFVLNNDGYTIERYLNVEISPDEQNYNDIPNWSYTKLAEAFGGELFTKTVRTNEELDEA
ITQAEQEYAEKLCLIEMIAADPMDAPEYMHRIRNHKQEQKK (SEQ ID NO: 16)
KivD (Identifier: tl63842; Accession: A0A0L7TB96)
ATGTCGACGACAACCGTTGGTGACTACTTGCTGTATCGCTTAAACGAAATCGGCATTGAGCACCTCTTCGGAGTG
CCAGGTGATTACAATCTGCAATTTCTGGATCATGTAATCGACCACCCTCAGCTGACTTGGGTCGGCTGCACTAAC
GAACTTAACGCTGCCTACGCAGCTGATGGTTATGCGCGTTGTCGTCCGGCTGCGGCACTGCTGACCACCTTCGGG
GTTGGCGAACTGAGCGCTATTAATGGCATCGCAGGTTCCTACGCGGAGTATCTGCCGGTAATACATATCGTTGGT
GCACCGAGTCTATCAGCCCAGCAGCAGGGCGACCTGATTCACCACTCTCTTGGCGAAGGTGATTTTTCCAGCTTC
CTGAGGATGTCCCAACCGGTGTCTGTTGCGCAGGCTGCTCTGACTCCTGATAACGCATGCAAGGAAATCGACCGC
GTACTGGCGGAAGTCCTCATTCAGCGTCGTCCCGGCTACCTGCTGCTGTCTACCGACGTGGCTGCTGCGCCGGCG
GCTCTGCCACAAAGCACTCTTTCTTTGCCGACCGCCCCGGATCATCGCGCAGTTCTGGCTGCTTTCAGCGACGCT
GCTGAGCAGATGCTGGCTCAGGCCAAAAGCGTCTCTCTACTGGCGGACTTTCTGGCTGATCGTTTCGGTGTTACT
CGAGCACTGGCCGCGTGGCTTCAGCAGGTTCCGCTACCGCACGCCACTCTGTTAATGGGTAAAGGCGTTCTGAGT
GAACAGCAACCAGGGTTCGTGGGTACCTACGCTGGTGCGGCATCTATCGATTCGACGCGTGGCGCAATCGAAGAA
GCTGGGGTAATTATCGGAGTGGGAGTTAGATTTTCCGACACTATCACAGCAGGCTTCTCGCAGCAGATCGACGCC
CGCCGTTTTATAGACATTCAACCCTTCTTCTCTCGTATTGGCGATCGCCAGTTTGATCACCTGCCGATGCAGGCT
GCCGTCGCAGCCCTGCATCAACTGTGTCTTCGTTATCAGCAGCAGTGGTCTATCACCGCTCCTAGCCCGCCTGCA
CTGCCGCCGGCTGCTGGTAGCGAGCTGTCCCAGAACGCATTCTGGCAGGCGATGCAGAACTTCATCCGCCCTGGG
GACCTGTTGGTGGCCGACCAAGGTACTGCGGCGTTCGGCGCAGCGGCGCTGCGCTTACCGCAGAATTGCCAGCTG
CTTGTGCAGCCGCTGTGGGGCTCAATCGGTTACAGTCTGCCGGCCACCTTTGGTGCTCAGACGGCAGATACAGAG
CGTCGTGTAATCCTAATCATTGGCGATGGTTCAGCGCAATTAACTATTCAGGAACTTTCCAGTATGATGCGTGAC
GGCTTGAAACCTATCATCTTTCTCCTGAACAACAACGGTTACACCGTTGAACGGGCGATTCACGGCGCGGAGCAA
CGTTATAACGATATCGCTGCTTGGAATTGGACCCAACTGCCCCAGGCGCTGAGTGTTCATTGCCCAGCGCAGAGC
TGGCGAGTCGTTGAAACGGTGCAGCTGACCGACGTAATGAAAGTCATCGCTGCTTCTCCGCGTCTGAGCTTGGTA
GAAGTTGTTCTGCCTGCAATGGATGTCCCACCGCTGCTGCAAGCAGTGAGTGCCGCTCTGAACCAGCGCAACTCC
TCT (SEQ ID NO: 17)
MSTTTVGDYLLYRLNEIGIEHLFGVPGDYNLQFLDHVIDHPQLTWVGCTNELNAAYAADGYARCRPAAALLTTFG
VGELSAINGIAGSYAEYLPVIHIVGAPSLSAQQQGDLIHHSLGEGDFSSFLRMSQPVSVAQAALTPDNACKEIDR
VLAEVLIQRRPGYLLLSTDVAAAPAALPQSTLSLPTAPDHRAVLAAFSDAAEQMLAQAKSVSLLADFLADRFGVT
RALAAWLQQVPLPHATLLMGKGVLSEQQPGFVGTYAGAASIDSTRGAIEEAGVIIGVGVRFSDTITAGFSQQIDA
RRFIDIQPFFSRIGDRQFDHLPMQAAVAALHQLCLRYQQQWSITAPSPPALPPAAGSELSQNAFWQAMQNFIRPG
DLLVADQGTAAFGAAALRLPQNCQLLVQPLWGSIGYSLPATFGAQTADTERRVILIIGDGSAQLTIQELSSMMRD
GLKPIIFLLNNNGYTVERAIHGAEQRYNDIAAWNWTQLPQALSVHCPAQSWRVVETVQLTDVMKVIAASPRLSLV
EVVLPAMDVPPLLQAVSAALNQRNSS (SEQ ID NO: 18)
Adh (Identifier: tl59319; Accession: A0A1E4TMA4)
ATGCAGACGGCGTTCTTGTATAAGCCAGGTCACGAAAACTTAGTGCGCTCGGAGATCCCGATACCTAAAGCTGGG
CGTGGCGAAGTCGTTCTGGAAATTAAAGCCGCTGGCATGTGCCATTCCGATCTGCACGTTCTCGACGGTGGAATC
CCCCTGCCGGGTCAATTTGTAATGGGCCATGAAATCGTTGGTACTATTCACGAGATCGGCCAGGACGTGACCGGT
TTCAAACAGGGCGATCTGTACGCAGTCCACGGCCCGAATCCGTGTGGTATTTGCACCCTGTGCAGAGAAGGATTT
GATAACGACTGCACTACAGTGGCGAAAACCGGTCAATGGTTCGGACTGGGTCTTGACGGCGGCTACCAGAAGTAT
ATCCGTATCCCGAACGTAAGGTCTATCGTTAAAGTTCCAGAAGGTGTTTCAGCTGAGGCAGCTGCGAGCTGTACT
GATGCAGTACTGACCCCGTACCGTGCACTAAAACAGGCTGGCGCCAGCAACTCTACTCGGGTACTGATTCTGGGT
CTGGGTGGCTTAGGTCTGAATGCCCTTAAACTGGCTAAGACCTTCGGCAGTTACGTTTACGCATCTGACCTGAAA
CCTTCTGCGCGTGAAGCTGCTAAGGCCGCTGGGGCGGATGAAGTGCTGGAGTCCCTGCCCGAAGACCCGCTGGGT
GTTGATATCGTGTTAGACGTCGTTGGCGTGCAGAGCACCTTCAACCTCGCTCAAAAACACGTTGGCCCGCGTGGC
ATCATTGTACCTGTAGGCCTGGCATCCCCACAGCTTTCGTTTAACCTAACGGATCTGGCGCTCCGCGAAATTCGT
GTTCAGGGCACTTTTTGGGGCACGAGCAATGAGCTGGCTGAATGTCTGCGCCTGTGCCAGCTGGGCCTGATCAAC
CCGAAATATACTGTGGTGCCTCTTGAAGAAGCGCCGAAATATATGGAAGCAATGGCTCATGGGAAAGTAGAAGGT
CGTATCGTTTTCCACCCG (SEQ ID NO: 19)
MQTAFLYKPGHENLVRSEIPIPKAGRGEVVLEIKAAGMCHSDLHVLDGGIPLPGQFVMGHEIVGTIHEIGQDVTG
FKQGDLYAVHGPNPCGICTLCREGFDNDCTTVAKTGQWFGLGLDGGYQKYIRIPNVRSIVKVPEGVSAEAAASCT
DAVLTPYRALKQAGASNSTRVLILGLGGLGLNALKLAKTFGSYVYASDLKPSAREAAKAAGADEVLESLPEDPLG
VDIVLDVVGVQSTFNLAQKHVGPRGIIVPVGLASPQLSFNLTDLALREIRVQGTFWGTSNELAECLRLCQLGLIN
PKYTVVPLEEAPKYMEAMAHGKVEGRIVFHP (SEQ ID NO: 20)
Adh (Identifier: tl59028; Accession: A0A192IDS9)
ATGCGCAGCATGCAGTTTGATGAGTACGGTGCACCCCTGAAAGCGTTCTCATATGAAGACCCGACCCCGCAAGGG
AAGGAAGTAGTCGTTAGGATCGAAGCCTGTGGTGTGTGCCACTCTGATATTCATCTTCACGAGGGCTACTTCGAC
ATGGGCGGTGGCAATAAAGCTGATGTTACTCGTGCTCGCGAACTCCCTTTTACATTGGGTCATGAAATCGTTGGC
GAAGTGGTAGCAACTGGACCAGGTGTCACCGGCGCTAAACCGGGCGACAAACGTATTGTGTACCCGTGGATCGGG
TGCGGCGACTGCCCGAAATGCAACAGTGGTGAGGATCAGTCCTGTGCGCGTCCACGTAACCTGGGTGTTCACGTT
GACGGTGGCTATTCGACGCACGTAAAGATACCGGACGAAAAATTCCTGTTCGCCTACGATGGTATTCCTACTGAG
TTAGCGGGAACCTATGCTTGCAGCGGCATCACCGCTTATGGTGCACTGATGAAAGCAAAGGAAGCGGCTGAAAGA
TCTGGCTACATCGGTCTGATTGGCGCTGGTGGCGTTGGCATGGCTGGTCTGATGCTGGCCAAAGCAGCGATCGGG
GCTAAAACTGTAGTCTTTGATATCGACGACGCAAAACTGGAAGCTGCGACCCGTGCCGGGGCGGATTACGTGTTC
AACTCCGGTGCAAAAGAAACACGCAAGGAAGTTATGAAACTAACGAATGGTGGCCTGTCTGGTGCTGTTGATTTC
GTTGGCAGCGATAAAAGCGCTCTGTTTGGAATCAACGCCTTGGGTCAGAACGGCGTGCTGGTCATAATTGGACTG
TTCGGTGGCGCTATGACTGTTCCGGTACCCCTGTTCCCGCTGAAAGGGATCACCGTACGTGGCTCATACGTAGGT
TCCCTGCAAGAGATGAGTGATATGATGGAGTTAGTTCGCGCTGGGAAAGTTCCTCCGATGCCGGTAAAAACTCGG
CCACTGGACGCTGCCTGGGAAACCCTTGAGGATCTACGCCATGGTAAAATCGTGGGCCGTGTTGTTCTGACCCCA
(SEQ ID NO: 21)
MRSMQFDEYGAPLKAFSYEDPTPQGKEVVVRIEACGVCHSDIHLHEGYFDMGGGNKADVTRARELPFTLGHEIVG
EVVATGPGVTGAKPGDKRIVYPWIGCGDCPKCNSGEDQSCARPRNLGVHVDGGYSTHVKIPDEKFLFAYDGIPTE
LAGTYACSGITAYGALMKAKEAAERSGYIGLIGAGGVGMAGLMLAKAAIGAKTVVFDIDDAKLEAATRAGADYVF
NSGAKETRKEVMKLTNGGLSGAVDFVGSDKSALFGINALGQNGVLVIIGLFGGAMTVPVPLFPLKGITVRGSYVG
SLQEMSDMMELVRAGKVPPMPVKTRPLDAAWETLEDLRHGKIVGRVVLTP (SEQ ID NO: 22)
Adh (Identifier: tl58538; Accession: A0A0P1J1W4)
ATGACAGCGGAGCAGCAAAATGGGGTATCCGACTCACGCCGTTTCGAATTTCAGGAATTTGGTGGCCCTATCGCC
CCACAGACCTATCAGCTCCCCGCACCGGCTAGCGATGAAGTTTTGTTAAAGGTGAACTACTGCGGTGTCTGTCAC
AGTGATGTTCATCTTCACGACGGCTACTTCGAGCTGGGTGGCGATAAACGTCTGAACTTCGCTATGCCGCTGCCG
CTGACGCTGGGTCACGAAGTAATTGGCACCGTTGTGGCTGTCGGCGACCAGGTTACTGGTGTAAAACCGGGGGAC
CAGCGACTGATCTATCCGTGGATAGGTTGCGGAAAATGCGGCGCGTGTCAAAAAGGAGAAGAAAACCTGTGCGTT
ACTCCTGCACATCTGGGCGTGAACAAGCCGGGCGGTTACGCTGATCACATCGTTGTACCCCATTCTCGCTACCTT
CTGGACATTTCGGGTCTGAACCCGGGTGATGCCGCTACCCTCGCGTGCTCCGGCCTGACCACTTTCAGCGCGATC
AACAAAGTGTTGCCGCTTGCAGATGACCAGTGGATTGTTGTTATCGGTTGTGGTGGCCTCGGCCAGATGGCGCTG
CGTATCCTGCAAGCTATGGGAATTGGCAATGTTATCGGTATTGACCTGTCTGAAGAGAAACGGAAACTGGCTCAT
GAAAGCGGTGCACGTCACTCCTTCGATCCAAACACTCCGAAGCTGAACCGCGTGGTCGCCGAAACCTGCCCGGGT
ACGGTACAGGCCGCGTTAGACTTTGTGGGCAATGAGCAAACTGCTCAGCTGGCACTGTCTCTGCTTGGAAAAGGT
GGCAAATATGTTCCTGTCGGGCTGCACGGCGGCGAGCTGCGTTACCCATTGCCGATCATCACGAACAAAGCTGTA
AGTATCATCGGTTCTTACGTTGGTACCCTGAAAGAACTGGAAGACTTAGTTGCTTTCGCCAAGGAAAAAAATCTG
CCGCCAATTCATATTGAACACCGCCCGCTGGAATCGGCGGCTCAGGCCGTAGAGGACCTGGAAAAAGGACAGGTT
GCTGGGCGTGTTATCCTGGATGCAGGTAAC(SEQ ID NO: 23)
MTAEQQNGVSDSRRFEFQEFGGPIAPQTYQLPAPASDEVLLKVNYCGVCHSDVHLHDGYFELGGDKRLNFAMPLP
LTLGHEVIGTVVAVGDQVTGVKPGDQRLIYPWIGCGKCGACQKGEENLCVTPAHLGVNKPGGYADHIVVPHSRYL
LDISGLNPGDAATLACSGLTTFSAINKVLPLADDQWIVVIGCGGLGQMALRILQAMGIGNVIGIDLSEEKRKLAH
ESGARHSFDPNTPKLNRVVAETCPGTVQAALDFVGNEQTAQLALSLLGKGGKYVPVGLHGGELRYPLPIITNKAV
SIIGSYVGTLKELEDLVAFAKEKNLPPIHIEHRPLESAAQAVEDLEKGQVAGRVILDAGN
(SEQ ID NO: 24)
GFP (Negative Control)
ATGACCGCACTTACGGAAGGGGCAAAACTGTTTGAGAAAGAGATACCGTATATAACCGAACTGGAAGGCGACGTA
GAAGGGATGAAATTTATAATTAAAGGCGAGGGGACCGGGGACGCGACCACGGGGACCATTAAAGCGAAATACATA
TGCACTACGGGCGACCTGCCGGTACCGTGGGCAACCCTGGTGAGCACCCTGAGCTACGGGGTCCAGTGTTTCGCC
AAGTACCCGAGCCACATAAAGGATTTCTTTAAGAGCGCCATGCCGGAAGGGTATACCCAAGAGCGTACCATAAGC
TTCGAAGGCGACGGCGTGTACAAGACGCGTGCTATGGTCACCTACGAACGCGGGTCTATATACAATCGTGTAACG
CTGACTGGGGAGAACTTTAAGAAAGACGGGCACATTCTGCGTAAGAACGTCGCATTCCAATGCCCGCCAAGCATT
CTGTATATTCTGCCTGACACCGTCAACAATGGCATACGCGTCGAGTTCAACCAGGCGTACGATATTGAAGGGGTG
ACCGAAAAACTGGTCACCAAATGCAGCCAAATGAATCGTCCGCTTGCGGGCAGTGCGGCAGTGCATATACCGCGT
TATCATCACATTACCTACCACACCAAACTGAGCAAAGACCGCGACGAGCGCCGTGATCACATGTGTCTGGTTGAG
GTAGTGAAAGCGGTCGATCTGGACACGTATCAGTGA (SEQ ID NO: 25)
MTALTEGAKLFEKEIPYITELEGDVEGMKFIIKGEGTGDATTGTIKAKYICTTGDLPVPWATLVSTLSYGVQCFA
KYPSHIKDFFKSAMPEGYTQERTISFEGDGVYKTRAMVTYERGSIYNRVTLTGENFKKDGHILRKNVAFQCPPSI
LYILPDTVNNGIRVEFNQAYDIEGVTEKLVTKCSQMNRPLAGSAAVHIPRYHHITYHTKLSKDRDERRDHMCLVE
WKAVDLDTYQ (SEQ ID NO: 26)

TABLE 4
Enzyme Screening Data
LeuDH enzymes and activity relative to control

| | | | Fold- | | |
|---|---|---|---|---|---|
| | | | Improvement | | |
| | Protein | | relative to | Nucleotide | Protein |
| Accession | Mutations | Strain | control | SEQ ID NO | SEQ ID NO |
| P0A392 | wt | Control | 0 | 37 | 257 |
| A0A1T4PGG9 | wt | t160946 | 2.846 | 38 | 258 |
| A4CBM3 | wt | t161014 | 2.188 | 39 | 259 |
| A0A0C1US13 | wt | t160854 | 2.178 | 40 | 260 |
| A0A1M6BE59 | wt | t160389 | 2.166 | 41 | 261 |
| K2M7H0 | wt | t160943 | 2.027 | 42 | 262 |
| A0A1Q6ZIF7 | wt | t160092 | 2.005 | 43 | 263 |
| A0A075JPW8 | wt | t160267 | 2.002 | 44 | 264 |
| A0A0B5AS65 | wt | t160288 | 1.910 | 45 | 265 |
| A0A0V8JFL2 | wt | t160337 | 1.826 | 46 | 266 |
| A0A1S2LUY1 | wt | t160524 | 1.804 | 47 | 267 |
| A0A0A8UN70 | wt | t161111 | 1.792 | 48 | 268 |
| P0A392 | G43T | t159984 | 1.775 | 49 | 269 |
| A0A1E7PTP0 | wt | t161162 | 1.751 | 50 | 270 |
| A0A1S9B636 | wt | t160283 | 1.741 | 51 | 271 |
| P0A392 | E116V | t160562 | 1.553 | 52 | 272 |
| A0A1D2RXB2 | wt | t160434 | 1.550 | 53 | 273 |
| K4KRS4 | wt | t160706 | 1.548 | 54 | 274 |
| P0A392 | L76F | t160502 | 1.538 | 55 | 275 |
| P0A392 | T136R | t160559 | 1.521 | 56 | 276 |
| P0A392 | A297C | t160202 | 1.509 | 57 | 277 |
| A0A1I1NGX1 | wt | t160947 | 1.501 | 58 | 278 |
| A0A142ITE6 | wt | t161198 | 1.401 | 59 | 279 |
| I1DTY5 | wt | t160169 | 1.364 | 60 | 280 |
| P0A392 | A297Y | t160199 | 1.364 | 61 | 281 |
| A0A0A0EMP0 | wt | t160499 | 1.359 | 62 | 282 |
| W4PY11 | wt | t160682 | 1.359 | 63 | 283 |
| R8B531 | wt | t161210 | 1.359 | 64 | 284 |
| A0A1Q2KY34 | wt | t160573 | 1.340 | 65 | 285 |
| L1QQC1 | wt | t161091 | 1.333 | 66 | 286 |
| D6XVM2 | wt | t160162 | 1.301 | 67 | 287 |
| P0A392 | L78V | t160587 | 1.281 | 68 | 288 |
| A0A1G8KLY7 | wt | t160351 | 1.267 | 69 | 289 |
| A0A0J6CNT2 | wt | t160438 | 1.254 | 70 | 290 |
| P0A392 | L300K | t160181 | 1.196 | 71 | 291 |
| U3HCY1 | wt | t161117 | 1.191 | 72 | 292 |
| A0A1K1TVW4 | wt | t160461 | 1.188 | 73 | 293 |
| A0A1Y6CWJ6 | wt | t160154 | 1.186 | 74 | 294 |
| A0A154W9T2 | wt | t160973 | 1.171 | 75 | 295 |
| I1D544 | wt | t161185 | 1.149 | 76 | 296 |
| A0A165NUD8 | wt | t161204 | 1.149 | 77 | 297 |
| A0A0A8JN83 | wt | t160338 | 1.144 | 78 | 298 |
| P0A392 | N71T | t160401 | 1.144 | 79 | 299 |
| F7RX04 | wt | t160786 | 1.110 | 80 | 300 |
| A0A1U9K9A9 | wt | t160671 | 1.108 | 81 | 301 |
| A0A0K6GVS2 | wt | t160957 | 1.105 | 82 | 302 |
| A0A136MKS4 | wt | t160417 | 1.095 | 83 | 303 |
| A0A0A5GIG6 | wt | t160609 | 1.076 | 84 | 304 |
| A0A143BJV1 | wt | t160627 | 1.051 | 85 | 305 |
| K6YKY7 | wt | t161088 | 1.046 | 86 | 306 |
| A0A0T5PG63 | wt | t160158 | 1.032 | 87 | 307 |
| A0A1M6L5E8 | wt | t160479 | 1.032 | 88 | 308 |
| P0A392 | L42Q | t160013 | 1.029 | 89 | 309 |
| A0A0A2TA47 | wt | t160286 | 1.017 | 90 | 310 |
| P0A392 | A297H | t160636 | 1.012 | 91 | 311 |
| A0A0Q5UT14 | wt | t160279 | 1.002 | 92 | 312 |
| I4D8U4 | wt | t160598 | 1.000 | 93 | 313 |
| P0A392 | I113V | t160129 | 0.993 | 94 | 314 |
| A0A1G3WLY4 | wt | t159999 | 0.976 | 95 | 315 |
| P0A392 | A297N | t160134 | 0.968 | 96 | 316 |
| P0A392 | A297M | t160503 | 0.954 | 97 | 317 |
| A0A1X4MV49 | wt | t160926 | 0.949 | 98 | 318 |
| P0A392 | A297L | t160497 | 0.912 | 99 | 319 |
| A0A0J1FEE3 | wt | t160141 | 0.897 | 100 | 320 |
| P0A392 | E116A | t160512 | 0.892 | 101 | 321 |
| P0A392 | M67T | t160125 | 0.883 | 102 | 322 |
| A0A0F7HKR2 | wt | t160291 | 0.873 | 103 | 323 |
| K0AAV5 | wt | t160552 | 0.870 | 104 | 324 |
| A0A1Q4XJW1 | wt | t160891 | 0.868 | 105 | 325 |
| P0A392 | L300N | t160557 | 0.866 | 106 | 326 |
| A0A0K9GVT6 | wt | t160443 | 0.863 | 107 | 327 |
| W7D8C3 | wt | t160771 | 0.858 | 108 | 328 |
| F7NG13 | wt | t160215 | 0.851 | 109 | 329 |
| A0A1H8Q403 | wt | t160870 | 0.836 | 110 | 330 |
| P0A392 | L42T | t160357 | 0.829 | 111 | 331 |
| E1WZZ8 | wt | t160664 | 0.797 | 112 | 332 |
| A0A0K9GC14 | wt | t160444 | 0.790 | 113 | 333 |
| P0A392 | V296N | t160184 | 0.787 | 114 | 334 |
| A0A1F3SFY8 | wt | t160002 | 0.785 | 115 | 335 |
| P0A392 | L78K | t160487 | 0.782 | 116 | 336 |
| P0A392 | T136S | t160176 | 0.768 | 117 | 337 |
| A0A1Y5EK08 | wt | t160841 | 0.768 | 118 | 338 |
| P0A392 | T136F | t160489 | 0.763 | 119 | 339 |
| N0AUJ4 | wt | t160823 | 0.751 | 120 | 340 |
| P0A392 | M67Q | t159980 | 0.748 | 121 | 341 |
| C4L3E4 | wt | t160256 | 0.748 | 122 | 342 |
| A0A1I6TTT1 | wt | t160115 | 0.733 | 123 | 343 |
| P0A392 | A297R | t160509 | 0.733 | 124 | 344 |
| A0A1H7JVK8 | wt | t160952 | 0.733 | 125 | 345 |
| A0A1U7M8J0 | wt | t160255 | 0.724 | 126 | 346 |
| P0A392 | L300Q | t160226 | 0.721 | 127 | 347 |
| A1S7B6 | wt | t160188 | 0.719 | 128 | 348 |
| P0A392 | V293S | t160602 | 0.711 | 129 | 349 |
| C1A7X5 | wt | t160733 | 0.709 | 130 | 350 |
| A0A0W0TJD2 | wt | t161212 | 0.697 | 131 | 351 |
| P0A392 | I113F | t160504 | 0.689 | 132 | 352 |
| P0A392 | M67E | t160064 | 0.685 | 133 | 353 |
| A0A1U7JH14 | wt | t160966 | 0.685 | 134 | 354 |
| P0A392 | L300A | t160612 | 0.680 | 135 | 355 |
| P0A392 | E116S | t160543 | 0.675 | 136 | 356 |
| P0A392 | G43F | t160059 | 0.672 | 137 | 357 |
| P0A392 | A297F | t160588 | 0.670 | 138 | 358 |
| M8DS05 | wt | t160310 | 0.663 | 139 | 359 |
| P0A392 | L300C | t160633 | 0.658 | 140 | 360 |
| P0A392 | L300F | t160128 | 0.655 | 141 | 361 |
| M7N8L2 | wt | t160152 | 0.655 | 142 | 362 |
| P0A392 | L78F | t160584 | 0.653 | 143 | 363 |
| G8R2S3 | wt | t160212 | 0.650 | 144 | 364 |
| A0A0P8B102 | wt | t161073 | 0.650 | 145 | 365 |
| S2YPJ0 | wt | t160830 | 0.643 | 146 | 366 |
| A0A1M5CX03 | wt | t159964 | 0.636 | 147 | 367 |
| P0A392 | L76E | t160245 | 0.626 | 148 | 368 |
| A0A1M5IEB6 | wt | t160988 | 0.626 | 149 | 369 |
| A0A0F6SHW7 | wt | t160860 | 0.619 | 150 | 370 |
| A0A0U3AUS4 | wt | t160964 | 0.619 | 151 | 371 |
| A0A081G3H3 | wt | t160968 | 0.604 | 152 | 372 |
| A0A1Q4UNH5 | wt | t161006 | 0.599 | 153 | 373 |
| P0A392 | A297D | t160548 | 0.597 | 154 | 374 |
| P0A392 | V293Q | t160249 | 0.594 | 155 | 375 |
| P0A392 | T136E | t160648 | 0.594 | 156 | 376 |
| P0A392 | L300D | t160248 | 0.587 | 157 | 377 |
| P0A392 | L300T | t160270 | 0.587 | 158 | 378 |
| P0A392 | L76H | t160546 | 0.587 | 159 | 379 |
| P0A392 | L76W | t160139 | 0.579 | 160 | 380 |
| P0A392 | L76M | t160274 | 0.575 | 161 | 381 |
| P0A392 | L300M | t160541 | 0.548 | 162 | 382 |
| T0CG61 | wt | t160808 | 0.538 | 163 | 383 |
| A0A166W971 | wt | t160538 | 0.535 | 164 | 384 |
| P0A392 | V296C | t160206 | 0.533 | 165 | 385 |
| P0A392 | A297E | t160567 | 0.533 | 166 | 386 |
| K2JU58 | wt | t160877 | 0.523 | 167 | 387 |
| P0A392 | G44I | t160011 | 0.516 | 168 | 388 |
| A0A0M4FMC6 | wt | t160371 | 0.516 | 169 | 389 |
| P0A392 | M67S | t160060 | 0.509 | 170 | 390 |
| A0A0K1JA83 | wt | t160995 | 0.509 | 171 | 391 |
| P0A392 | A115T | t159988 | 0.504 | 172 | 392 |
| A0A1N6U8W9 | wt | t160814 | 0.504 | 173 | 393 |
| A0A075LQK1 | wt | t160493 | 0.499 | 174 | 394 |
| P0A392 | G44Y | t160080 | 0.494 | 175 | 395 |
| P0A392 | L300H | t160197 | 0.494 | 176 | 396 |
| A0A0K8QRE8 | wt | t160626 | 0.489 | 177 | 397 |
| A0A1M6M3I5 | wt | t160012 | 0.487 | 178 | 398 |
| A0A0F7JZ22 | wt | t161016 | 0.477 | 179 | 399 |
| P0A392 | L78H | t160634 | 0.469 | 180 | 400 |
| A0A1Y6BX33 | wt | t160700 | 0.460 | 181 | 401 |
| P0A392 | V296L | t160146 | 0.447 | 182 | 402 |
| A0A1L8CTI5 | wt | t161020 | 0.445 | 183 | 403 |
| P0A392 | L300Y | t160145 | 0.443 | 184 | 404 |
| P0A392 | E116N | t160539 | 0.428 | 185 | 405 |
| A0A171DN74 | wt | t160716 | 0.423 | 186 | 406 |
| P0A392 | A297K | t160491 | 0.416 | 187 | 407 |
| P0A392 | L78Y | t160594 | 0.416 | 188 | 408 |
| E6TXR8 | wt | t160618 | 0.416 | 189 | 409 |
| P0A392 | N71H | t160120 | 0.411 | 190 | 410 |
| A0A1G3X1T7 | wt | t160910 | 0.411 | 191 | 411 |
| P0A392 | E116W | t160246 | 0.408 | 192 | 412 |
| U4KND6 | wt | t160852 | 0.408 | 193 | 413 |
| P0A392 | E116R | t160131 | 0.399 | 194 | 414 |
| P0A392 | N71C | t160385 | 0.399 | 195 | 415 |
| A0A1G0BBA9 | wt | t160899 | 0.396 | 196 | 416 |
| A0A1Y2L717 | wt | t160990 | 0.396 | 197 | 417 |
| P0A392 | A297T | t160227 | 0.389 | 198 | 418 |
| A0A0M4UKZ2 | wt | t160340 | 0.379 | 199 | 419 |
| P0A392 | A297W | t160596 | 0.357 | 200 | 420 |
| P0A392 | L78C | t160406 | 0.350 | 201 | 421 |
| E2SC01 | wt | t161059 | 0.350 | 202 | 422 |
| A0A1K1PP57 | wt | t160629 | 0.347 | 203 | 423 |
| P0A392 | G44K | t159990 | 0.345 | 204 | 424 |
| P0A392 | A115S | t160495 | 0.342 | 205 | 425 |
| P0A392 | L300S | t160275 | 0.337 | 206 | 426 |
| P0A392 | L300W | t160639 | 0.337 | 207 | 427 |
| A0A1G0A9I7 | wt | t160875 | 0.337 | 208 | 428 |
| A0A0W7WYJ8 | wt | t161047 | 0.337 | 209 | 429 |
| P0A392 | V296E | t160520 | 0.325 | 210 | 430 |
| P0A392 | T136Y | t160638 | 0.325 | 211 | 431 |
| P0A392 | A115V | t160123 | 0.320 | 212 | 432 |
| A0A1V0ADI4 | wt | t160970 | 0.318 | 213 | 433 |
| W7ZGF1 | wt | t160812 | 0.315 | 214 | 434 |
| P0A392 | A115Q | t159982 | 0.311 | 215 | 435 |
| A0A1H6CJX7 | wt | t161141 | 0.308 | 216 | 436 |
| P0A392 | M67K | t160356 | 0.296 | 217 | 437 |
| P0A392 | L78Q | t160581 | 0.296 | 218 | 438 |
| P0A392 | T136L | t160589 | 0.293 | 219 | 439 |
| P0A392 | E116L | t160604 | 0.293 | 220 | 440 |
| P0A392 | I113M | t160628 | 0.291 | 221 | 441 |
| P0A392 | L76Y | t160516 | 0.289 | 222 | 442 |
| P0A392 | V293A | t160655 | 0.274 | 223 | 443 |
| P0A392 | V296K | t160243 | 0.267 | 224 | 444 |
| P0A392 | L76R | t160153 | 0.264 | 225 | 445 |
| P54531 | wt | t160721 | 0.262 | 226 | 446 |
| P0A392 | V296I | t160271 | 0.259 | 227 | 447 |
| P0A392 | L300R | t160560 | 0.254 | 228 | 448 |
| K9ARW8 | wt | t160789 | 0.252 | 229 | 449 |
| P0A392 | L76S | t160133 | 0.249 | 230 | 450 |
| P0A392 | I113W | t160094 | 0.244 | 231 | 451 |
| P0A392 | A115N | t160194 | 0.240 | 232 | 452 |
| P0A392 | V296S | t160644 | 0.240 | 233 | 453 |
| P0A392 | E116M | t160643 | 0.235 | 234 | 454 |
| P0A392 | L42A | t160402 | 0.232 | 235 | 455 |
| P0A392 | V293C | t160500 | 0.225 | 236 | 456 |
| P0A392 | N71M | t160324 | 0.220 | 237 | 457 |
| P0A392 | V296A | t160143 | 0.213 | 238 | 458 |
| P0A392 | G43W | t160099 | 0.210 | 239 | 459 |
| P0A392 | A297Q | t160140 | 0.196 | 240 | 460 |
| P0A392 | V293T | t160221 | 0.191 | 241 | 461 |
| P0A392 | I113Y | t160098 | 0.188 | 242 | 462 |
| P0A392 | L76I | t160601 | 0.188 | 243 | 463 |
| P0A392 | G44H | t160029 | 0.176 | 244 | 464 |
| P0A392 | L76K | t160585 | 0.171 | 245 | 465 |
| P0A392 | G43Y | t159996 | 0.169 | 246 | 466 |
| P0A392 | N71D | t160415 | 0.142 | 247 | 467 |
| P0A392 | I113Q | t160632 | 0.139 | 248 | 468 |
| P0A392 | M67A | t160055 | 0.127 | 249 | 469 |
| P0A392 | V296T | t160630 | 0.122 | 250 | 470 |
| P0A392 | L76T | t160603 | 0.115 | 251 | 471 |
| A0A1Q4VRJ4 | wt | t161033 | 0.112 | 252 | 472 |
| B2A513 | wt | t160167 | 0.108 | 253 | 473 |
| P0A392 | G43E | t160096 | 0.083 | 254 | 474 |
| P0A392 | N71K | t160101 | 0.044 | 255 | 475 |

TABLE 5
KivD enzymes and activity relative to control

| | | Fold- | | |
|---|---|---|---|---|
| | | Improvement | | |
| | | compared to | Nucleotide | Protein |
| Accession | Label | control | SEQ ID NO: | SEQ ID NO |
| Q684J7 | Control | 0 | 477 | 533 |
| A0A085UD38 | t163850 | 1.958 | 478 | 534 |
| A0A090DYV6 | t163542 | 3.986 | 479 | 535 |
| A0A0A6W4H3 | t163732 | 4.354 | 480 | 536 |
| A0A0B1U4F6 | t163805 | 2.972 | 481 | 537 |
| A0A0D0SDJ9 | t163730 | 3.292 | 482 | 538 |
| A0A0D2CSK3 | t163274 | 3.965 | 483 | 539 |
| A0A0D2GWW0 | t163016 | 4.354 | 484 | 540 |
| A0A0H4KFT8 | t163716 | 3.958 | 485 | 541 |
| A0A0J8UR79 | t163869 | 2.250 | 486 | 542 |
| A0A0K2Y209 | t163916 | 3.944 | 487 | 543 |
| A0A0L0P8D8 | t163988 | 5.097 | 488 | 544 |
| A0A0L7TB96 | t163842 | 4.833 | 489 | 545 |
| A0A0M5JJZ2 | t164076 | 4.944 | 490 | 546 |
| A0A0M5MY84 | t163914 | 4.139 | 491 | 547 |
| A0A0Q4N500 | t164007 | 4.493 | 492 | 548 |
| A0A0T9T7Y7 | t163705 | 3.694 | 493 | 549 |
| A0A0T9UPI9 | t163338 | 3.493 | 494 | 550 |
| A0A0U1CW59 | t163964 | 3.201 | 495 | 551 |
| A0A0U2NS09 | t163656 | 2.222 | 496 | 552 |
| A0A198FEB4 | t163871 | 4.382 | 497 | 553 |
| A0A1B1NY37 | t163888 | 3.646 | 498 | 554 |
| A0A1B7ILY5 | t163742 | 3.792 | 499 | 555 |
| A0A1B9AUW4 | t162995 | 3.889 | 500 | 556 |
| A0A1D4X3F2 | t163818 | 4.708 | 501 | 557 |
| A0A1F2KK66 | t163546 | 3.403 | 502 | 558 |
| A0A1G7WAJ7 | t163085 | 5.076 | 503 | 559 |
| A0A1M7EHD4 | t163474 | 1.813 | 504 | 560 |
| A0A1Q4T3V5 | t163704 | 4.535 | 505 | 561 |
| A0A1T1GFV6 | t163784 | 3.500 | 506 | 562 |
| A0A1U4TJK1 | t163702 | 4.722 | 507 | 563 |
| A0A1V2L8B3 | t164100 | 3.229 | 508 | 564 |
| A0A1V2YXQ3 | t163766 | 2.319 | 509 | 565 |
| A0A1V4SV36 | t163852 | 4.396 | 510 | 566 |
| A0A1V6TQU7 | t162902 | 3.118 | 511 | 567 |
| A0A1W6B724 | t163806 | 3.639 | 512 | 568 |
| A0A1X0AE10 | t163798 | 3.104 | 513 | 569 |
| A0A1X1XPA7 | t163472 | 3.826 | 514 | 570 |
| A0A1X2FKJ1 | t163432 | 3.035 | 515 | 571 |
| A0A1Y6E4E9 | t163406 | 3.486 | 516 | 572 |
| A0A205J7X5 | t163837 | 3.910 | 517 | 573 |
| A0A2B1L7A1 | t163722 | 4.215 | 518 | 574 |
| B9DJU8 | t163844 | 4.597 | 519 | 575 |
| D4B725 | t163868 | 4.111 | 520 | 576 |
| D4C3A5 | t163661 | 2.139 | 521 | 577 |
| D4F0I3 | t163478 | 0.896 | 522 | 578 |
| D7UWC4 | t163880 | 3.785 | 523 | 579 |
| F5SQV4 | t163740 | 2.535 | 524 | 580 |
| G9YCD8 | t163678 | 0.090 | 525 | 581 |
| I1CGS4 | t163934 | 3.785 | 526 | 582 |
| J2LV57 | t163902 | 2.667 | 527 | 583 |
| Q6C9L5 | t163155 | 4.222 | 528 | 584 |
| R5SST3 | t163337 | 3.014 | 529 | 585 |
| R8AV71 | t163285 | 4.535 | 530 | 586 |
| S3IST7 | t163983 | 2.979 | 531 | 587 |
| W0L941 | t163973 | 3.396 | 532 | 588 |

TABLE 6
Adh enzymes and activity relative to control

| | | Fold- | Nucleotide | Protein |
|---|---|---|---|---|
| | | Improvement | Sequence | Sequence |
| Accession | Label | relative to control | SEQ ID NO | SEQ ID NO |
| P00331 | Control | 0 | 589 | 645 |
| A0A011RFM0 | t159061 | −0.581 | 590 | 646 |
| A0A068NM64 | t159163 | 4.815 | 591 | 647 |
| A0A081B9F7 | t159282 | 5.992 | 592 | 648 |
| A0A0F7S860 | t159174 | −0.411 | 593 | 649 |
| A0A0F8XA97 | t159080 | −0.250 | 594 | 650 |
| A0A0L8BIH2 | t158526 | 0.629 | 595 | 651 |
| A0A0M2SIC1 | t158995 | 0.323 | 596 | 652 |
| A0A0M8TKC3 | t158267 | 2.427 | 597 | 653 |
| A0A0N1F703 | t159004 | 9.032 | 598 | 654 |
| A0A0P1J1W4 | t158538 | 10.516 | 599 | 655 |
| A0A0Q6FH05 | t159022 | −0.113 | 600 | 656 |
| A0A0Q9AMT3 | t158946 | 1.476 | 601 | 657 |
| A0A163KUH6 | t159154 | 0.710 | 602 | 658 |
| A0A192IDS9 | t159028 | 10.581 | 603 | 659 |
| A0A1A0K0C6 | t159162 | 0.645 | 604 | 660 |
| A0A1E4TMA4 | t159319 | 11.113 | 605 | 661 |
| A0A1E7X363 | t159283 | 4.234 | 606 | 662 |
| A0A1Q7HM90 | t159036 | −0.492 | 607 | 663 |
| A0A1V1TTZ9 | t158998 | −0.613 | 608 | 664 |
| A0A1V2EYM1 | t159040 | 3.750 | 609 | 665 |
| A0A1V6E459 | t159120 | 1.008 | 610 | 666 |
| A0A1Y0G594 | t159236 | 1.645 | 611 | 667 |
| A2V8B3 | t159176 | 4.758 | 612 | 668 |
| A9MKQ8 | t158774 | −0.548 | 613 | 669 |
| C0SPA5 | t158820 | 1.113 | 614 | 670 |
| D8MZF3 | t159280 | 6.234 | 615 | 671 |
| F0IX07 | t159318 | 0.371 | 616 | 672 |
| H1ZV38 | t158442 | 1.694 | 617 | 673 |
| J1KN15 | t158976 | 4.008 | 618 | 674 |
| J5T2P7 | t159183 | −0.161 | 619 | 675 |
| K4IPR3 | t158247 | 5.444 | 620 | 676 |
| M1LUC5 | t158246 | 0.073 | 621 | 677 |
| M2N9N4 | t159152 | 0.669 | 622 | 678 |
| M2QHN1 | t159090 | 0.282 | 623 | 679 |
| M2YNQ9 | t159054 | 3.629 | 624 | 680 |
| M5FVU5 | t158291 | 0.565 | 625 | 681 |
| O74822 | t158955 | −0.500 | 626 | 682 |
| P08843 | t158458 | 0.460 | 627 | 683 |
| P0DMQ6 | t158893 | −0.444 | 628 | 684 |
| P13603 | t158263 | 0.645 | 629 | 685 |
| P14219 | t158869 | −0.048 | 630 | 686 |
| P14673 | t158726 | 0.952 | 631 | 687 |
| P14675 | t158728 | 6.056 | 632 | 688 |
| P20368 | t158816 | 0.798 | 633 | 689 |
| P25141 | t158333 | 3.677 | 634 | 690 |
| P28032 | t158454 | 2.887 | 635 | 691 |
| P39451 | t158390 | 5.500 | 636 | 692 |
| P39849 | t158243 | 0.516 | 637 | 693 |
| P40394 | t158613 | 3.460 | 638 | 694 |
| P42328 | t158520 | 2.065 | 639 | 695 |
| Q2FJ31 | t158326 | 1.024 | 640 | 696 |
| Q38707 | t158580 | −0.105 | 641 | 697 |
| Q99W07 | t158330 | 1.597 | 642 | 698 |
| S0EJ18 | t159328 | 11.185 | 643 | 699 |
| W5YKG3 | t159122 | 0.782 | 644 | 700 |

TABLE 7
Conserved amino acids in enzymes with increased LeuDH activity
relative to SEQ ID NO: 27.

| Corresponding Position in | |
|---|---|
| SEQ ID NO: 27 | Amino Acid |
| 13 | V |
| 16 | W |
| 42 | Q |
| 43 | T, Y, F, E, W |
| 44 | I, H, K, Y |
| 67 | T, E, A, S, K |
| 71 | K |
| 73 | S |
| 76 | R, H, Y, S, K, W |
| 92 | Y |
| 93 | H |
| 95 | G |
| 100 | G |
| 105 | C |
| 111 | G |
| 113 | M |
| 115 | N, V |
| 116 | R, N, W |
| 120 | A |
| 122 | D |
| 136 | E |
| 140 | D |
| 141 | M |
| 160 | S |
| 185 | F |
| 196 | N |
| 228 | Y |
| 248 | M |
| 256 | C |
| 293 | Q, C |
| 296 | K, N |
| 297 | R, Q, K |
| 300 | C, D |
| 302 | T, S |
| 305 | C |
| 319 | F |
| 330 | M |

TABLE 8
Conserved amino acids in enzymes with increased KivD activity
relative to SEQ ID NO: 29.

| Corresponding Position in | |
|---|---|
| Position in SEQ ID NO: 29 | Amino Acid |
| 33 | Y |
| 44 | Q |
| 117 | M |
| 129 | I |
| 185 | W |
| 190 | I |
| 225 | I |
| 227 | Y |
| 311 | L |
| 312 | G |
| 313 | T |
| 328 | P |
| 341 | W |
| 345 | H |
| 347 | C |
| 420 | R |
| 494 | D |
| 508 | C |
| 550 | F |

TABLE 9
Conserved amino acids in enzymes with increased ADH activity
relative to SEQ ID NO: 31.

| Corresponding Position | |
|---|---|
| in SEQ ID NO: 31 | Amino Acid |
| 9 | P |
| 16 | G |
| 23 | Q |
| 28 | R |
| 30 | A |
| 93 | K |
| 98 | L |
| 99 | R |
| 114 | P |
| 115 | K |
| 119 | Y |
| 194 | Y |
| 242 | P |
| 249 | K |
| 255 | E |
| 260 | D |
| 269 | H |
| 281 | Q |
| 325 | L |
| 333 | M |
| 334 | P |
| 348 | Q |

### EQUIVALENTS

Those skilled in the art will recognize, or be able to ascertain using no more than routine experimentation, many equivalents to the specific embodiments of the invention described in this disclosure. Such equivalents are intended to be encompassed by the following claims.

All references, including patent documents, disclosed in this application are incorporated by reference in their entirety, particularly for the disclosure referenced in this disclosure.

What is claimed is:

1. A host cell that comprises a heterologous polynucleotide encoding a leucine dehydrogenase (LeuDH) enzyme, wherein the LeuDH enzyme comprises an amino acid sequence that is at least 90% identical to a sequence selected from SEQ ID NOs: 2, 4, 6, 8, 10, and 12.

2. The host cell of claim 1, wherein the LeuDH enzyme comprises an amino acid sequence that is at least 90% identical to SEQ ID NO: 2.

3. The host cell of claim 2, wherein the LeuDH enzyme comprises SEQ ID NO: 2.

4. The host cell of claim 1 or 2, wherein the LeuDH enzyme comprises:
a) V at a residue corresponding to residue 13 in SEQ ID NO: 27;
b) W at a residue corresponding to residue 16 in SEQ ID NO: 27;
c) Q at a residue corresponding to residue 42 in SEQ ID NO: 27;
d) T, Y, F, E, or W at a residue corresponding to residue 43 in SEQ ID NO: 27;
e) I, H, K, or Y at a residue corresponding to residue 44 in SEQ ID NO: 27;
f) T, E, A, S, or K at a residue corresponding to residue 67 in SEQ ID NO: 27;
g) K at a residue corresponding to residue 71 in SEQ ID NO: 27;
h) S at a residue corresponding to residue 73 in SEQ ID NO: 27;
i) R, H, Y, S, K, or W at a residue corresponding to residue 76 in SEQ ID NO: 27;
j) Y at a residue corresponding to residue 92 in SEQ ID NO: 27;
k) H at a residue corresponding to residue 93 in SEQ ID NO: 27;
l) G at a residue corresponding to residue 95 in SEQ ID NO: 27;
m) G at a residue corresponding to residue 100 in SEQ ID NO: 27;
n) C at a residue corresponding to residue 105 in SEQ ID NO: 27;
o) G at a residue corresponding to residue 111 in SEQ ID NO: 27;
p) M at a residue corresponding to residue 113 in SEQ ID NO: 27;
q) N or V at a residue corresponding to residue 115 in SEQ ID NO: 27;
r) R, N, or W at a residue corresponding to residue 116 in SEQ ID NO: 27;
s) A at a residue corresponding to residue 120 in SEQ ID NO: 27;
t) D at a residue corresponding to residue 122 in SEQ ID NO: 27;
u) E at a residue corresponding to residue 136 in SEQ ID NO: 27;
v) D at a residue corresponding to residue 140 in SEQ ID NO: 27;
w) M at a residue corresponding to residue 141 in SEQ ID NO: 27;
x) S at a residue corresponding to residue 160 in SEQ ID NO: 27;
y) F at a residue corresponding to residue 185 in SEQ ID NO: 27;
z) N at a residue corresponding to residue 196 in SEQ ID NO: 27;
aa) Y at a residue corresponding to residue 228 in SEQ ID NO: 27;
bb) M at a residue corresponding to residue 248 in SEQ ID NO: 27;
cc) C at a residue corresponding to residue 256 in SEQ ID NO: 27;
dd) Q or C at a residue corresponding to residue 293 in SEQ ID NO: 27;
ee) K or N at a residue corresponding to residue 296 in SEQ ID NO: 27;
ff) R, Q, or K at a residue corresponding to residue 297 in SEQ ID NO: 27;
gg) C or D at a residue corresponding to residue 300 in SEQ ID NO: 27;
hh) T or S at a residue corresponding to residue 302 in SEQ ID NO: 27;
ii) C at a residue corresponding to residue 305 in SEQ ID NO: 27;
jj) F at a residue corresponding to residue 319 in SEQ ID NO: 27; and/or
kk) M at a residue corresponding to residue 330 in SEQ ID NO: 27.

5. The host cell of claim 4, wherein the LeuDH enzyme comprises all of (a)-(kk).

6. A host cell that comprises a heterologous polynucleotide encoding a leucine dehydrogenase (LeuDH) enzyme, wherein relative to SEQ ID NO: 27, the LeuDH enzyme comprises an amino acid substitution at amino acid residue: 42, 43, 44, 67, 71, 76, 78, 113, 115, 116, 136, 293, 296, 297 and/or 300.

7. The host cell of claim 6, wherein the LeuDH enzyme comprises:
a) A, Q, or T at residue 42;
b) E, F, T, W, or Y at residue 43;
c) H, I, K, or Y at residue 44;
d) A, E, K, Q, S, or T at residue 67;
e) C, D, H, K, M, or Tat residue 71;
f) E, F, H, I, K, M, R, S, T, W, or Y at residue 76;
g) C, F, H, K, Q, V, or Y at residue 78;
h) F, M, Q, V, W, or Y at residue 113;
i) N, Q, S, T, or V at residue 115;
j) A, L, M, N, R, S, V, or W at residue 116;
k) E, F, L, R, S, or Y at residue 136;
l) A, C, Q, S, or T at residue 293;
m) A, C, E, I, K, L, N, S, or T at residue 296;
n) C, D, E, F, H, K, L, M, N, Q, R, T, W, or Y at residue 297; and/or
o) A, C, D, F, H, K, M, N, Q, R, S, T, W, or Y at residue 300.

8. A non-naturally occurring LeuDH enzyme, wherein relative to SEQ ID NO: 27, the LeuDH enzyme comprises an amino acid substitution at amino acid residue:
42, 43, 44, 67, 71, 76, 78, 113, 115, 116, 136, 293, 296, 297 and/or 300.

9. The non-naturally occurring LeuDH enzyme of claim 8, wherein the LeuDH enzyme comprises:
a) A, Q, or T at residue 42;
b) E, F, T, W, or Y at residue 43;
c) H, I, K, or Y at residue 44;
d) A, E, K, Q, S, or T at residue 67;
e) C, D, H, K, M, or Tat residue 71;
f) E, F, H, I, K, M, R, S, T, W, or Y at residue 76;
g) C, F, H, K, Q, V, or Y at residue 78;
h) F, M, Q, V, W, or Y at residue 113;
i) N, Q, S, T, or V at residue 115;
j) A, L, M, N, R, S, V, or W at residue 116;
k) E, F, L, R, S, or Y at residue 136;
l) A, C, Q, S, or T at residue 293;
m) A, C, E, I, K, L, N, S, or T at residue 296;
n) C, D, E, F, H, K, L, M, N, Q, R, T, W, or Y at residue 297; and/or
o) A, C, D, F, H, K, M, N, Q, R, S, T, W, or Y at residue 300.

10. A host cell that comprises a heterologous polynucleotide encoding a branched chain α-ketoacid decarboxylase (KivD) enzyme, wherein the KivD enzyme comprises an amino acid sequence that is at least 90% identical to a sequence selected from SEQ ID NOs: 14, 16, and 18.

11. The host cell of claim 10, wherein the KivD enzyme comprises an amino acid sequence that is at least 90% identical to SEQ ID NO: 18.

12. The host cell of claim 11, wherein the KivD enzyme comprises SEQ ID NO: 18.

13. The host cell of claim 10 or 11, wherein the KivD enzyme comprises:
a) Y at a residue corresponding to residue 33 in SEQ ID NO: 29;
b) Q at a residue corresponding to residue 44 in SEQ ID NO: 29;
c) M at a residue corresponding to residue 117 in SEQ ID NO: 29;
d) I at a residue corresponding to residue 129 in SEQ ID NO: 29;
e) W at a residue corresponding to residue 185 in SEQ ID NO: 29;
f) I at a residue corresponding to residue 190 in SEQ ID NO: 29;
g) I at a residue corresponding to residue 225 in SEQ ID NO: 29;
h) Y at a residue corresponding to residue 227 in SEQ ID NO: 29;
i) L at a residue corresponding to residue 311 in SEQ ID NO: 29;
j) G at a residue corresponding to residue 312 in SEQ ID NO: 29;
k) T at a residue corresponding to residue 313 in SEQ ID NO: 29;
l) P at a residue corresponding to residue 328 in SEQ ID NO: 29;
m) W at a residue corresponding to residue 341 in SEQ ID NO: 29;
n) H at a residue corresponding to residue 345 in SEQ ID NO: 29;
o) C at a residue corresponding to residue 347 in SEQ ID NO: 29;
p) R at a residue corresponding to residue 420 in SEQ ID NO: 29;
q) D at a residue corresponding to residue 494 in SEQ ID NO: 29;
r) C at a residue corresponding to residue 508 in SEQ ID NO: 29; and/or
s) F at a residue corresponding to residue 550 in SEQ ID NO: 29.

14. The host cell of claim 13, wherein the KivD enzyme comprises all of (a)-(s).

15. A host cell that comprises a heterologous polynucleotide encoding a an alcohol dehydrogenase (Adh) enzyme wherein the Adh enzyme comprises an amino acid sequence that is at least 90% identical to a sequence selected from SEQ ID NOs: 20, 22, and 24.

16. The host cell of claim 15, wherein the Adh enzyme comprises an amino acid sequence that is at least 90% identical to SEQ ID NO: 24.

17. The host cell of claim 16, wherein the Adh enzyme comprises SEQ ID NO: 24.

18. The host cell of claim 15 or 16, wherein the Adh enzyme comprises:
a) P at a residue corresponding to residue 9 in SEQ ID NO: 31;
b) G at a residue corresponding to residue 16 in SEQ ID NO: 31;
c) Q at a residue corresponding to residue 23 in SEQ ID NO: 31;
d) R at a residue corresponding to residue 28 in SEQ ID NO: 31;
e) A at a residue corresponding to residue 30 in SEQ ID NO: 31;
f) K at a residue corresponding to residue 93 in SEQ ID NO: 31;
g) L at a residue corresponding to residue 98 in SEQ ID NO: 31;
h) R at a residue corresponding to residue 99 in SEQ ID NO: 31;
i) P at a residue corresponding to residue 114 in SEQ ID NO: 31;
j) K at a residue corresponding to residue 115 in SEQ ID NO: 31;
k) Y at a residue corresponding to residue 119 in SEQ ID NO: 31;
l) Y at a residue corresponding to residue 194 in SEQ ID NO: 31;
m) P at a residue corresponding to residue 242 in SEQ ID NO: 31;
n) K at a residue corresponding to residue 249 in SEQ ID NO: 31;
o) E at a residue corresponding to residue 255 in SEQ ID NO: 31;
p) D at a residue corresponding to residue 260 in SEQ ID NO: 31;
q) H at a residue corresponding to residue 269 in SEQ ID NO: 31;
r) Q at a residue corresponding to residue 281 in SEQ ID NO: 31;
s) L at a residue corresponding to residue 325 in SEQ ID NO: 31;
t) M at a residue corresponding to residue 333 in SEQ ID NO: 31;
u) P at a residue corresponding to residue 334 in SEQ ID NO: 31; and/or
v) Q at a residue corresponding to residue 348 in SEQ ID NO: 31.

19. The host cell of claim 18, wherein the Adh enzyme comprises all of (a)-(v).

20. The host cell of any one of claims 1-7 and 10-19, wherein the host cell is a plant cell, an algal cell, a yeast cell, a bacterial cell, or an animal cell.

21. The host cell of claim 20, wherein the host cell is a yeast cell.

22. The host cell of claim 21, wherein the yeast cell is an Saccharomyces cell, a Yarrowia cell or a Pichia cell.

23. The host cell of claim 20, wherein the host cell is a bacterial cell.

24. The host cell of claim 23, wherein the bacterial cell is an E. coli cell or a Bacillus cell.

25. The host cell of any one of claims 1-7 and 10-24, wherein the host cell further comprises a heterologous polynucleotide encoding a Branched-chain amino acid transport system 2 carrier protein (BrnQ).

26. The host cell of claim 25, wherein the BrnQ protein is at least 90% identical to the amino acid sequence of SEQ ID NO: 35.

27. The host cell of any one of claims 1-7 and 10-26, wherein the heterologous polynucleotide is operably linked to an inducible promoter.

28. The host cell of any one of the claims 1-7 and 10-27, wherein the heterologous polynucleotide is expressed in an operon.

29. The host cell of claim 28, wherein the operon expresses more than one heterologous polynucleotide and wherein a ribosome binding site is present between each heterologous polynucleotide.

30. The host cell of any one of claims 1-7, wherein the host cell further comprises a heterologous polynucleotide encoding a KivD enzyme and/or a heterologous polynucleotide encoding an Adh enzyme.

31. The host cell of any one of claims 10-14, wherein the host cell further comprises a heterologous polynucleotide encoding a LeuDH enzyme and/or a heterologous polynucleotide encoding an Adh enzyme.

32. The host cell of any one of claims 15-19, wherein the host cell further comprises a heterologous polynucleotide encoding a LeuDH enzyme and/or a heterologous polynucleotide encoding a KivD enzyme.

33. The host cell of any one of claims 1-7 and 10-32, wherein the host cell is capable of producing isopentanol from leucine.

34. The host cell of claim 33, wherein the host cell consumes at least two-fold more leucine relative to a control host cell that comprises a heterologous polynucleotide encoding a control LeuDH enzyme comprising the sequence of SEQ ID NO: 27, a heterologous polynucleotide encoding a control KivD enzyme comprising the sequence of SEQ ID NO: 29, a heterologous polynucleotide encoding a control Adh enzyme comprising the sequence of SEQ ID NO: 31, and a heterologous polynucleotide encoding a control BrnQ protein comprising the sequence of SEQ ID NO: 35.

35. A method comprising culturing the host cell of any one of claims 1-7 and 10-34.

36. A method for producing isopentanol from leucine comprising culturing the host cell of any one of claims 1-7 and 10-34.

37. A non-naturally occurring nucleic acid comprising a sequence that is at least 90% identical to a nucleic acid sequence selected from SEQ ID NOs: 1, 3, 5, 7, 9, and 11.

38. A non-naturally occurring nucleic acid comprising a sequence that is at least 90% identical to a nucleic acid sequence selected from SEQ ID NOs: 13, 15, and 17.

39. A non-naturally occurring nucleic acid comprising a sequence that is at least 90% identical to a nucleic acid sequence selected from SEQ ID NOs: 19, 21, and 23.

40. A non-naturally occurring nucleic acid encoding a sequence that is at least 90% identical to a sequence selected from SEQ ID NOs: 2, 4, 6, 8, 10, and 12.

41. A non-naturally occurring nucleic acid encoding a sequence that is at least 90% identical to a sequence selected from SEQ ID NOs: 14, 16, and 18.

42. A non-naturally occurring nucleic acid encoding a sequence that is at least 90% identical to a sequence selected from SEQ ID NOs: 20, 22, and 24.

43. A vector comprising the non-naturally occurring nucleic acid of any one of claims 37-42.

44. An expression cassette comprising the non-naturally occurring nucleic acid of any one of claims 37-42.
